(19) United States
(12) Patent Application Publication
(10) Pub. No.: US 2025/0009253 A1
(43) Pub. Date: Jan. 9, 2025

(54) DIAGNOSTIC PLATFORM FOR TESTING EXHALED BREATH CONDENSATE AND UNIVERSAL BIOSENSOR

(71) Applicants: John J. Daniels, Madison, CT (US); Diagmetrics, Inc., Madison, CT (US)

(72) Inventors: John J. Daniels, Madison, CT (US); Shekhar WADEKAR, Waltham, MA (US)

(21) Appl. No.: 18/443,286

(22) Filed: Feb. 15, 2024

Related U.S. Application Data
(63) Continuation of application No. PCT/US22/76511, filed on Sep. 15, 2022.
(60) Provisional application No. 63/245,295, filed on Sep. 17, 2021.
(60) Provisional application No. 63/233,473, filed on Aug. 16, 2021.

(51) Int. Cl. A61B 5/08; A61B 5/00; A61B 5/097; G01N 33/497

(52) U.S. Cl. CPC A61B 5/082; A61B 5/097; A61B 5/6803; G01N 33/497; G01N 2800/12

(57) ABSTRACT

A mask-based diagnostic apparatus for detecting a biomarker contained in exhaled breath of a test subject. An exhaled breath condensate (EBC) collector converts breath vapor from the lungs and airways into a fluid biosample. The EBC collector includes a condensate-forming surface and a thermal mass in thermal connection with the condensate-forming surface. A fluid transfer system transfers the EBC to at least one of a testing unit and an EBC containment vessel. Prior to testing, a semipermeable membrane concentrates a target biomarker portion in the fluid biosample to form a concentrated fluid biosample for testing. A target biomarker releasing material, such as a lysing agent, and/or mechanical lysing, can be used to obtained a target biomarker for testing that is within a viral envelope. The testing unit can be a universal g-FET biosensor constructed as a packaged semiconductor device that receives and tests the EBC as a fluid biosample.

### CROSS-REFERENCE TO RELATED APPLICATIONS

This is a continuation of PCT application PCT/US22/76511, filed Sep. 15, 2022, for Diagnostic Platform For Testing Exhaled Breath Condensate And Universal Biosensor, which claims the benefit of priority of (i) U.S. provisional patent application No. 63/245,295, invented by Daniels, et al., filed on Sep. 17, 2021, for Diagnostic Platform For Testing Exhaled Breath Condensate And Universal Biosensor, and (ii) U.S. provisional patent application No. 63/233,473, invented by Daniels, filed on Aug. 16, 2021, for Diagnostic Platform for Testing Exhaled Breath Condensate. The disclosures of all of the foregoing applications are incorporated by reference herein in their entireties.

### BACKGROUND OF THE INVENTION

The exemplary and non-limiting embodiments of this invention relate generally to diagnostic systems, methods, devices and computer programs and, more specifically, relate to digital and analog diagnostic devices for detecting a biomarker of a biological agent such as a coronavirus, lung cancer, tuberculosis, asthma, and other respiratory ailments and conditions, and/or blood borne biomarkers and other biomarkers that are present in the exhaled breath of a test subject.

The present invention also pertains to a device architecture, specific-use applications, and computer algorithms used to detect biometric parameters for the treatment and monitoring of physiological conditions in humans and animals.

This section is intended to provide a background or context to the exemplary embodiments of the invention as recited in the claims. The description herein may include concepts that could be pursued but are not necessarily ones that have been previously conceived, implemented or described.

Therefore, unless otherwise indicated herein, what is described in this section is not prior art to the description and claims in this application and is not admitted to being prior art by inclusion in this section.

Testing for biomarkers that indicate exposure, infection, progression and recovery from a disease condition, such as COVID-19 can be used to screen individuals for infection and help slow the spread of the virus. For example, protein and RNA testing for active virus shows who is currently contagious. Antibody testing can be used to find the members of a population that have recovered from the virus.

Diagnostics of SARS-CoV-2 infection using real-time reverse-transcription polymerase chain reaction (RT-PCR) on nasopharyngeal swabs is now well-established, with saliva-based testing being lately more widely implemented for being more adapted for self-testing approaches. The procedure to obtain nasal swab samples is not only uncomfortable, but requires specialized personal with risk of contaminating the person performing the test. Saliva tests have the advantage of being simpler to perform, less invasive with limited risks and RT-PCR on saliva specimens has becoming more widely implemented. The viscose nature of saliva together with the presence of saliva proteases, responsible for the proteolytic activity of saliva, make the direct application of saliva samples challenging. It is well known that the major mechanisms of COVID-19 spread are airborne and contact infections primarily due aerosol droplets expelled from the lungs and airways of infected persons. There is therefore a growing need for sample collection by patients themselves and a simple to use testing system that can detect a target biomarker indicative of a pathogenic infection from a biosample obtained from the lungs and airways.

### SUMMARY OF THE INVENTION

The below summary section is intended to be merely exemplary and non-limiting. The foregoing and other problems are overcome, and other advantages are realized, by the use of the exemplary embodiments of this invention.

In accordance with a non-limiting exemplary embodiment, an exhaled breath condensate (EBC) collector converts breath vapor received from the lungs and airways of the test subject into a fluid biosample. The EBC collector includes a condensate-forming surface and a thermal mass in thermal connection with the condensate-forming surface. A fluid transfer system transfers the EBC to at least one of a testing unit and an EBC containment vessel.

In accordance with another non-limiting exemplary embodiment, an electronic biosensor comprises a substrate having a water absorbing property provided by at least one of a selectively permeable membrane, a super absorbent polymer, a microfluidic material, and a wick. At least two electrodes are formed on a top surface of the substrate defining a gap there between. A functionalized detector provided in the gap comprises an electron transport material and a capture molecule. A target molecule captured by the capture molecule causes a change in at least one of a polarity and conductivity of the electron transport material. The target molecule is detected by testing for the change in the polarity or conductivity of the electron transport material. The target molecule that is detected depends on the capture molecule, so this electronic biosensor construction can be utilized to test for many different diseases and other use-cases by changing the capture molecule to match a known biomarker for a corresponding disease or use-case.

In accordance with another non-limiting exemplary embodiment, an apparatus for testing exhaled breath condensate (EBC) for a target biomarker includes an EBC collector for converting breath vapor received from the lungs and airways of a test subject into an EBC biosample. The EBC biosample contains the target analyte. A biomarker concentrator includes a super absorbent polymer layer in a flow path of the EBC biosample. During the collection, the EBC biosample is contacted with the super absorbent polymer layer and a portion of water from the EBC biosample is absorbed. The super absorbent polymer does not absorb the target analyte resulting in an increased concentration of the target analyte in the remaining water in the EBC fluid biosample. A biomarker testing unit receives the concentrated EBC biosample and tests the concentrated EBC biosample for the target biomarker (target molecule or target analyte).

In accordance with another aspect of the invention, a method for concentrating a target analyte in an exhaled breath condensate (EBC) sample comprises the steps of collecting the EBC sample from the lungs and airways of a test subject. The EBC contains the target analyte. A super absorbent polymer is provided in a flow path of the EBC where during the collection, the EBC sample is contacted with the super absorbent polymer. The super absorbent polymer absorbs a portion of water from the EBC sample and does not absorb the target analyte resulting in a concentration of the target analyte in remaining water in the EBC sample.

In accordance with another aspect of the invention, a capture molecule conjugate detects a target analyte. An applied-field-reactive capture molecule conjugate has at least one applied-field-responsive end and at least one capture molecule end. Each said capture molecule end binds to and captures the target analyte. The binding of each said capture molecule end to the target analyte increases an electrical charge difference between the at least one applied-field-responsive end and the at least one capture molecule end.

In accordance with another aspect of the invention, a method for detecting a target analyte comprises the steps of providing a capture molecule structure having a ligand end and a polarizable end. When the capture molecule structure is disposed in a carrier fluid, the capture molecule structure is a free-floating element. A target analyte as another free-floating element in the carrier fluid. The ligand end of the capture molecule structure binds to the target analyte and forms a free-floating polar conjugate having a positive end and a negative end. To test for the target analyte, the polar conjugate is aligned in the carrier fluid in an electric field and an electrical property of the aligned polar conjugate is measured.

In accordance with another aspect of the invention, a method is provided for forming a condensate collector having fluid conductor channels on a substrate for guiding a flow of fluid towards a desired direction. The substrate has a surface having a relatively lower energy surface property. A textured structure is formed on the surface to form fluid conductor channels having a relatively higher energy surface property for guiding a flow of fluid towards a desired direction.

In accordance with another aspect of the invention, a method for assembling an array of applied-field-reactive capture molecule conjugates comprises the steps of providing dissolvable adhesive film. A carrier fluid that is a non-solvent for the dissolvable adhesive film is provided. The carrier fluid has randomly dispersed applied-field-reactive capture molecule conjugates. An aligning field is applied to the carrier fluid for assembling the applied-field-reactive capture molecule conjugates onto the dissolvable adhesive film. The carrier fluid is removed by evaporation and/or another process, leaving the assembled applied-field-reactive capture molecule conjugates fixed on the dissolvable adhesive film.

In accordance with another non-limiting exemplary embodiment, a testing system includes an exhaled breath condensate (EBC) collector for converting breath vapor received from the lungs and airways of a test subject into a fluid biosample. A biomarker concentrator concentrates a target biomarker portion in the fluid biosample to form a concentrated fluid biosample. A biomarker testing unit receives the concentrated fluid biosample and test the concentrated fluid biosample for the target biomarker.

In accordance with another non-limiting exemplary embodiment, an exhaled breath condensate (EBC) collector converts breath vapor received from the lungs and airways of the test subject into a fluid biosample. The EBC collector includes a condensate-forming surface with a thermal mass in thermal connection with the condensate-forming surface. A fluid transfer system transfers the EBC to at least one of a testing unit and an EBC containment vessel.

In accordance with another non-limiting exemplary embodiment, a method for forming a condensate collector having fluid conductor channels on a substrate for guiding a flow of fluid towards a desired direction comprises the steps of providing the substrate having a surface having a relatively lower energy surface property, and forming a textured structure to form fluid conductor channels on the surface having a relatively higher energy surface property for guiding a flow of fluid towards a desired direction.

In accordance with another non-limiting exemplary embodiment, an apparatus comprises at least one Processor, at least one Memory including computer program code, the at least one Memory and the computer program code configured to, with the at least one Processor, cause the apparatus to perform at least the following: providing a capture molecule structure having a ligand end and a polarizable end, wherein when the capture molecule structure is disposed in a carrier fluid, the capture molecule structure is a free floating element; providing a target analyte as another free floating element in the carrier fluid, where the ligand end of the capture molecule structure binds to the target analyte and forms a free floating polar conjugate having a positive end and a negative end; and testing an electrical property of the aligned polar conjugate to detect the target analyte.

In accordance with another non-limiting exemplary embodiment, a computer program product comprising a computer-readable medium bearing computer program code embodied therein for use with a computer, the computer program code comprising: code for: testing an electrical property of an aligned polar conjugate to detect a target analyte by providing a capture molecule structure having a ligand end and a polarizable end, wherein when the capture molecule structure is disposed in a carrier fluid, the capture molecule structure is a free floating element, and the target analyte is provided as another free floating element in the carrier fluid, where the ligand end of the capture molecule structure binds to the target analyte and forms the free floating polar conjugate having a positive end and a negative end.

In accordance with another non-limiting embodiment, a sensor for detecting target molecules in a fluid sample comprises a detection area for receiving the fluid sample comprising the target molecules. The detection area includes a detection interface functionalized with capture molecules. A top driving electrode and a bottom driving electrode define a gap there between. A fluid conductor disposed in the gap conducts the fluid sample through the gap. An electric potential applied to the top and the bottom driving electrode drives the target molecules towards the capture molecules to concentrate the target molecules in a portion of the fluid sample received at the detection interface.

In accordance with another non-limiting embodiment, a method for detecting a target molecule from a fluid sample comprises the steps of: receiving the fluid sample comprising the target molecule in a microfluidic channel; transferring the fluid sample from the microfluidic channel to a detection interface of a sensor, the sensor comprising a detection area for receiving the fluid sample and having the detection interface functionalized with capture molecules, a top driving electrode and a bottom driving electrode defining a gap there between, and a fluid conductor disposed in the gap for conducting the fluid sample through the gap. An electric potential applied to the top and the bottom driving electrode drives the target molecules towards the capture molecules to concentrate the target molecules in a portion of the fluid sample received at the detection interface. The sensor may comprise a field-effect transistor having a gate disposed in electrical communication with the detection interface functionalized with the capture molecules, and a source and drain on either side of the gate. At least a portion of at least one of the top and the bottom driving electrode is disposed in the detection area, where a gate electrode of the sensor comprises at least one of the top and the bottom driving electrode. The electric potential drives the target molecules towards the capture molecules and is cyclically and intermittently applied along with taking a test reading of a change in an electrical characteristic at the source, drain and gate caused by the capturing of the target molecules.

In accordance with another non-limiting exemplary embodiment, a room scale biosensor comprises an intake for taking in ambient air. A condenser cools the ambient air to condense moisture vapor to a condensate containing water and at least one target molecule. A condensate testing system tests the condensate for the at least one target molecule. The condensate testing system includes a biosensor comprising at least one g-FET biosensor having a detection interface comprising a graphene layer functionalized with capture molecules, where the capture molecules are smaller than the Debye screening length.

In accordance with another non-limiting exemplary embodiment, a field-effect transistor sensor circuit is provided for detecting target molecules in a fluid sample. A semiconductor substrate of one conductivity type has a source region and a drain region defining there between a channel region of the one conductivity type. The source region and the drain region are of an opposite conductivity type to the substrate and channel regions. An insulator is formed over the channel region, where the channel region forms a back gate of the field-effect transistor. A detection area is formed over the insulator for receiving the fluid sample. A detection interface is functionalized with capture molecules. A top electrode defines a gap with the detection area and also forms a liquid gate electrode of the transistor. A fluid conductor is disposed in the gap for conducting the fluid sample through the gap. A driving circuit applies an electric potential of one polarity to the top electrode and of the opposite polarity to the back gate. The electric potential drives the target molecules towards the capture molecules to concentrate the target molecules in a portion of the fluid sample received at the detection interface.

In accordance with another non-limiting exemplary embodiment, a packaged semiconductor device includes a universal biosensor provided from a g-FET die singulated from a wafer and encapsulated in epoxy. A detection well is formed in molded epoxy top and disposed over the graphene detection interface so the fluid sample can reach the immobilized capture molecules at the detection interface. The graphene layer may be formed and functionalized with the capture molecules at the wafer level. During fabrication, the functionalized graphene is protected with a dissolvable mask left in place so the die can be picked and placed, and mounted on a lead frame then encapsulated in epoxy with the detection well either preformed in an epoxy cap or formed in place during encapsulation.

### BRIEF DESCRIPTION OF THE DRAWINGS

To easily identify the discussion of any particular element or act, the most significant digit or digits in a reference number refer to the figure number in which that element is first introduced.

FIG. 1 shows a face mask and schematically illustrates an Exhaled Breath Condensate (EBC) collector, fluid transfer system, biosensor testing system and near field communication (NFC) antenna and signal condition circuit all disposed within the confined volume of the inside of the face mask.

FIG. 2 shows the inside of the mask splayed open with components for collecting and testing EBC and exhaled breath aerosols (EBA).

FIG. 3 is a block diagram of one possible and non-limiting exemplary system in which some of the exemplary embodiments may be practiced.

FIG. 4 is a logic flow diagram for Applied Probabilistic Analysis to determine the detecting of a target biomarker, and illustrates the operation of an exemplary method, a result of execution of computer program instructions embodied on a computer readable Memory, functions performed by logic implemented in hardware, and/or interconnected means for performing functions in accordance with exemplary embodiments.

FIG. 5 is a logic flow diagram for Data Acquisition and Transmission for Trusted Receiver and Population Study use-cases, and illustrates the operation of an exemplary method, a result of execution of computer program instructions embodied on a computer readable Memory, functions performed by logic implemented in hardware, and/or interconnected means for performing functions in accordance with exemplary embodiments.

FIG. 6 is a block diagram of the basic components for testing EBC and transmitting the test result to a smartphone and/or cloud server.

FIG. 7 shows a KN95 face mask with a retrofittable testing system including an EBC collector having an aluminum foil condensate-forming surface, embossed fluid conductor channels, a fluid transfer system and electronic biosensor electrodes.

FIG. 8 shows the testing system retrofit into the KN95 mask.

FIG. 9 shows the KN95 mask having removable testing and communication electronics disposed on the outside of the mask.

FIG. 10 shows an Exhaled Breath Condensate Collector and a testing area with ganged syndromic testing biosensors.

FIG. 11 shows a pooling area for immersing the biosensors in collected EBC.

FIG. 12 shows an assembled EBC collector and pooling area.

FIG. 13 shows the sizes of an engineered capture molecule, an S-protein, a virus particle and a water molecule.

FIG. 14 shows an EBC concentrator having a semipermeable membrane for separating excess water from EBC with concentrated virus particles.

FIG. 15 shows the addition of a super absorbent polymer in the EBC concentrator.

FIG. 16 shows the addition of a surfactant or lysing agent in the EBC concentrator.

FIG. 17 shows an EBC droplet having concentrated virus particles and lysed proteins.

FIG. 18 is a cross section showing an EBC concentrator with a semipermeable membrane and wick disposed adjacent to a thermal mass.

FIG. 19 is a cross section showing an EBC concentrator with a fluid conductor with lysing material and having a semipermeable membrane and wick in an EBC pooling area.

FIG. 20 illustrates a capture molecule/linker/magnetic nanoparticle conjugate with a free floating and captured target molecule.

FIG. 21 illustrates an EBC collector with oriented and aligned magnetic nanoparticle conjugates on a dissolvable adhesive for capturing and concentrating target molecules in exhaled breath vapor.

FIG. 22 illustrates the EBC collector with oriented and aligned magnetic nanoparticle conjugates with EBC formed placing the target molecules in binding communication with the capture molecules.

FIG. 23 illustrates the water in the EBC dissolving the dissolvable adhesive allowing magnetically active molecule bound to target molecule conjugates to flow towards a magnetic trap at a testing area.

FIG. 24 illustrates the magnetically active molecule bound to target molecule conjugates held at the magnetic trap at the testing area.

FIG. 25 shows a dissolvable adhesive film supporting a carrier fluid holding free floating and randomly dispersed magnetic nanoparticle conjugates before a magnetic alignment field is applied, where the carrier fluid is a non-solvent for the dissolvable adhesive.

FIG. 26 shows the magnetic nanoparticle conjugates oriented and aligned on the dissolvable adhesive film in the carrier fluid holding free floating conjugates after a magnetic alignment field is applied.

FIG. 27 shows the well-organized magnetic nanoparticle conjugates fixed in place on the dissolvable adhesive film after the carrier fluid is allowed to evaporate.

FIG. 28 shows endothermic reaction constituents.

FIG. 29 shows a retrofittable endothermic EBC collector and a pre-existing face mask.

FIG. 30 shows an endothermic EBC collector having hydrophilic channels on a hydrophobic field.

FIG. 31 shows an endothermic EBC collector retrofit into an existing face mask.

FIG. 32 shows a reacted endothermic EBC collector and collected EBC droplets.

FIG. 33 shows an endothermic EBC collector with a pooling area and dry buffer/surfactant.

FIG. 34 shows an Exhaled Breath Condensate Collector and a testing area with ganged syndromic testing biosensors.

FIG. 35 shows a pooling area for immersing the biosensors in collected EBC, with dry buffer/surfactant in the pooling area.

FIG. 36 shows an assembled EBC collector and pooling area with dry buffer/surfactant in the pooling area.

FIG. 37 shows a Teflon condensing surface with a textured fluid conductor.

FIG. 38 is a photomicrograph showing tunable water adhesion structures fabricated by laser ablation.

FIG. 39 illustrates the tunable water adhesion structures formed in a Teflon sheet and having Teflon “spikes” for mechanically disrupting a viral envelope to produce free floating N- and S-proteins of a corona virus.

FIG. 40 shows a roll-to-roll process for mass producing a condensing surface with a textured fluid conductor.

FIG. 41 shows a sheet of condensing surfaces with textured fluid conductors.

FIG. 42 shows a capture molecule structure having a label attached to a ligand.

FIG. 43 shows a capture molecule structure having a polarizable end and a ligand end for capturing a target analyte.

FIG. 44 shows the capture molecule structure with a captured target analyte forming a polar conjugate.

FIG. 45 shows the alignment and testing of the polar conjugate to detect the target analyte in a fluid sample.

FIG. 46 shows a carrier fluid comprising a fluid sample with free floating target analyte molecules mixed with capture molecule structures to form free-floating polar conjugates in the fluid sample.

FIG. 47 shows the free-floating polar conjugates aligning in an applied electric field.

FIG. 48 shows a polarized gold nanoparticle/linker/capture molecule/target molecule conjugate.

FIG. 49 shows a polarized gold nanoparticle/linker/capture molecule conjugate where the linker is effective to functionalize the gold nanoparticle to form a positive gold nanoparticle end.

FIG. 50 shows a polarized gold nanoparticle/linker/capture molecule conjugate where the linker is effective to functionalize the gold nanoparticle to form a negative gold nanoparticle end.

FIG. 51 schematically shows a testing system where EBC containing a target molecule is mixed with a polarizable capture molecule conjugate to form a polarized conjugate, and the polarized conjugate is aligned in a gap between electrodes and the electrical characteristics of the aligned polarized conjugate is used to test for the presence of the target molecule in the EBC.

FIG. 52 graphically shows the test results of a proof-of-concept laboratory engineered mask and EBC collection system.

FIG. 53 shows an EBC based diagnostic strategy for testing for SARS-CoV-2 infectivity.

FIG. 54 illustrates a 2D aptamer capture molecule structure that was experimentally shown to be effective in the EBC based diagnostic strategy for testing for SARS-CoV-2 infectivity.

FIG. 55 illustrates biolayer interferometry (BLI) measurements of an aptamer linked onto activated BLI sensors with different concentrations of SARS-CoV-2 S protein.

FIG. 56 illustrates a surface attachment strategy of a SARS-Cov-2 aptamer on a gold working electrode of a screen-printed electrode.

FIG. 57 illustrates cyclic voltammograms (CV) of a gold electrode functionalized with SARS-CoV-2 aptamer using ferrocenemethanol as a redox mediator.

FIG. 58 hows (a) a differential pulse voltammogram of an aptamer electrochemical SARS-CoV-2 aptasensor, (b) current response to increasing binding domain concentrations, (c) Langmuir adsorption isotherm, (d) response curve of different virus variants, and (e) the selectivity of the aptamer sensors.

FIG. 59 is a table showing exploratory EBC studies on patients identified by nasopharyngeal swabs RT-PCR as SARS-CoV-2 positive or negative.

FIG. 60 illustrates an applied-field-reactive capture molecule conjugate having an applied-field-responsive end and a capture molecule end with a linker molecule providing electro-chemical properties that change at least one of a polarity and a conductivity.

FIG. 61 illustrates a capture molecule conjugate having a magnetically attractive end and a capture molecule conjugate having a coated AuNP end.

FIG. 62 shows a process for forming aligned and oriented capture molecule conjugates aligned in a magnetic field on a dissolvable adhesive.

FIG. 63 shows a process for forming aligned and oriented capture molecule conjugates aligned in an electric field on a dissolvable adhesive.

FIG. 64 shows a Lateral Flow Assay (LFA) testing system showing a biomarker (analyte) sample added to a sample pad with a magnetically attractive label linked to a capture molecule aligned and oriented on a dissolvable adhesive film formed on a conjugate release pad.

FIG. 65 shows the LFA with a analyte-labeled capture molecule complex formed at the conjugate release pad.

FIG. 66 shows the binding of the biomarker at a test line indicating the presence of the biomarker.

FIG. 67 shows another testing system that can be used with the inventive EBC collection system that uses an electronic biosensor.

FIG. 68 shows a biosensor constructed on a water absorbing substrate comprising at least one of a selectively permeable membrane and super absorbent polymer fibers, where excess water in an fluid sample are absorbed by the substrate leaving behind a greater concentration of target biomarkers in the tested fluid sample.

FIG. 69 shows a sample pad of a microfluidic testing system, such as a lateral flow assay, with a target molecule concentrating structure comprising at least one of a selectively permeable membrane, a SAP/wick and a dissolvable fluid dam.

FIG. 70 shows a flow-through sensor electrode construction having one or more capture molecules functionalized on an electron transfer constituent, such as carbon nanotubes, where the electrodes are formed on a water absorbing substrate comprised of a selectively permeable membrane and SAP/capillary fiber composite wick.

FIG. 71 is a flow chart showing the steps of concentrating a target biomarker in an EBC fluid biosample using an electronic biosensor comprising a substrate having a water absorbing property provided by at least one of a selectively permeable membrane (SPM), a super absorbent polymer, water conducting fibers, a microfluidic material, and a wick.

FIG. 72 shows an electronic biosensor with a detection interface constructed on a carbon nano-tube immobilized (CNIM) super-absorbent polymer structure for concentrating a target biomarker in an EBC fluid biosample.

FIG. 73 shows an exploded view of a target molecule concentring flow-through electrode structure with electrodes formed on a water proof superstrate with a drainage hole in the area of a detection interface.

FIG. 74 shows the flow path of a fluid sample with the target biomarker blocked at the sensor electrodes and excess water flowing past the electrodes.

FIG. 75 shows a nanoCLAMP capture molecule immobilized on a magnetically active nanoparticle with two capture molecules binding with different binding sites of a protein molecule.

FIG. 76 shows two gold nanoparticles with immobilized nanoCLAMP capture molecules binding with the same target protein molecule.

FIG. 77 hows the relative sizes of a nanoCLAMP and classical antibody capture molecule compared to the Debye length.

FIG. 78 shows a monolayer of graphene functionalized with nanoCLAMP capture molecules through a pyrene linker, where example capture molecules (e.g., two or more nanoCLAMP capture molecules) are binding to different binding sites of an example target molecule (e.g., a protein).

FIG. 79 is a cross-section view showing a biosensor having an electric field applying structure for concentrating target molecules received at a detection interface, and a local heating element provided at the biosensor for speeding a binding reaction between the capture molecules and the target molecules.

FIG. 80 is an enlarged view showing an electric field driving charged target molecules towards capture molecules at a detection interface.

FIG. 81 is an isolated view showing SAP beads held on a top driving electrode grid for absorbing excess water from a fluid sample.

FIG. 82 schematically shows a structure of a field-effect transistor sensor.

FIG. 83 schematically shows a structure of the field-effect transistor sensor with electric filed applying bottom driving electrode grid that also acts as a liquid gate electrode for the field-effect transistor sensor.

FIG. 84 shows a fluid conductor applied on top of the bottom driving electrode grid/gate electrode.

FIG. 85 shows a top driving electrode disposed on the fluid conductor.

FIG. 86 shows SAP beads for removing excess water from the fluid sample held on the top driving electrode grid.

FIG. 87 shows a wick in fluid communication with the SAP beads for removing excess water from the fluid sample.

FIG. 88 is a side view of a sensor for detecting target molecules in a fluid sample with a detection area for receiving the fluid sample and a top driving electrode and a bottom driving electrode where an electric potential drives the target molecules towards the capture molecules to concentrate the target molecules in a portion of the fluid sample received at the detection interface.

FIG. 89 is a flowchart of the steps for concentrating the target molecules in a fluid sample and testing for a change in electrical characteristics of a functionalized transistor sensor.

FIG. 90 is a side view of a back gated field-effect transistor sensor with a detection area for receiving a fluid sample and a top driving electrode working in cooperation with a back gate electrode for applying an electric potential that drives the target molecules towards the capture molecules to concentrate the target molecules in a portion of the fluid sample received at the detection interface.

FIG. 91 is a flowchart of the steps for using an applied electric field for concentrating target molecules and testing for a change in electrical characteristics of a functionalized field-effect transistor sensor.

FIG. 92 is a side view of a sensor for detecting target molecules in a fluid sample with a detection area for receiving a portion of the fluid sample downstream from where a top driving electrode and a bottom driving electrode are provided to apply an electric potential to facilitate separating the target molecules from excess water in the fluid sample prior to the tested sample entering the detection interface.

FIG. 93 schematically shows a room scale biosensor for detecting target molecules in a fluid sample obtained from ambient humidity pulled from the room.

FIG. 94 is a block diagram showing the components of an embodiment of the room scale biosensor.

FIG. 95 is an illustration of a field-effect transistor biosensor with a fluid sample flow conductor and capture molecules standing off from the conductive surface of a detection interface at a length less than the Debye screening length.

FIG. 96 is an illustration of the field-effect transistor biosensor showing a fluid sample flow direction with randomly dispersed target molecules and ions in a flow conductor above the detection interface.

FIG. 97 is an illustration of a field-effect transistor sensor sensing circuit for detecting target molecules in a fluid sample showing a driving circuit for driving the target molecules towards the capture molecules immobilized on the detection interface.

FIG. 98 is an illustration of a field-effect transistor sensor sensing circuit for detecting target molecules in a fluid sample showing a liquid gate detecting circuit for detecting the presence and quantity of target molecules captured at the detection interface.

FIG. 99 is an illustration of a field-effect transistor sensor sensing circuit for detecting target molecules in a fluid sample showing a back gate detecting circuit for detecting the presence and quantity of target molecules captured at the detection interface.

FIG. 100 illustrates a graphene detection interface with nanoCLAMP capture molecules immobilized by linker molecules, with a portion of the capture molecules immobilized at a greater distance from the detection interface than another portion of the capture molecules.

FIG. 101 illustrates an electric field potential applied in the detection area and driving a target molecule and non-target molecule towards the capture molecules, where the target molecule is captured by a capture molecule extending on linker molecules at a relatively longer distance from the detection interface.

FIG. 102 illustrates the electric field potential removed from detection area.

FIG. 103 illustrates an opposite polarity electric field potential that drives the target molecule and non-target molecule away from the detection interface, where the target molecule remains tethered by the capture molecule immobilized by the relatively long linker on the detection interface.

FIG. 104 illustrates more target molecules and non-target molecules flowing into the detection area.

FIG. 105 illustrates the electric field potential applied in the detection area, driving the tethered target molecule into position to be captured by a capture molecule immobilized by a relatively shorter linker molecule, and where another target molecule is captured by another capture molecule extending on linker molecules at a relatively longer distance from the detection interface to concentrate the target molecules in a portion of the fluid sample received at the detection interface.

FIG. 106 is a block diagram of an g-FET sensor integrated circuit having a functionalizable detection interface.

FIG. 107 shows ganged g-FET sensors fabricated in the functionalizable detection interface.

FIG. 108 shows a g-FET semiconductor device encapsulated in an epoxy base with the detection interface of the g-FET exposed at the surface of the epoxy base.

FIG. 109 shows an epoxy top with a detection well opening disposed over the epoxy base with the detection interface accessible through the detection well opening.

FIG. 110 shows a fluid conductor in fluid communication with the detection interface through the detection well.

FIG. 111 shows a mesh driving electrode/liquid gate electrode disposed on the fluid conductor.

FIG. 112 shows SAP provided on the top of the mesh driving electrode/liquid gate electrode.

FIG. 113 shows a wick disposed over the SAP and the mesh driving electrode/liquid gate electrode.

FIG. 114 shows a packaged g-FET semiconductor device encapsulated in an epoxy with source, gate and drain leads.

FIG. 115 shows a z-axis conductor for connecting the source, gate and drain leads of the packaged g-FET semiconductor device to an external electrical circuit.

FIG. 116 shows the packaged g-FET semiconductor device connected to source, gate and drain circuit lines of an external electrical circuit.

FIG. 117 shows the packaged g-FET semiconductor device having a driving electrode/liquid gate lead disposed for connecting the mesh driving electrode/liquid gate electrode to a lead line of an external electrical circuit.

FIG. 118 is a wireframe of the packaged g-FET semiconductor device showing the driving electrode/liquid gate lead disposed on the top of the epoxy top and the bottom of the epoxy base for connecting the mesh driving electrode/liquid gate electrode to a lead line of an external electrical circuit.

FIG. 119 shows the packaged g-FET semiconductor device having the driving electrode/liquid gate lead disposed on top of the fluid conductor and connecting the top of there fluid conductor to a lead line of an external electrical circuit.

FIG. 120 shows the bottom of the packaged g-FET semiconductor device showing the source and drain leads, and the back gate and driving electrode/liquid gate leads.

### DETAILED DESCRIPTION

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The word “exemplary” is used herein to mean “serving as an example, instance, or illustration.” Any embodiment described herein as “exemplary” is not necessarily to be construed as preferred or advantageous over other embodiments. In any case, all of the embodiments described in this Detailed Description are exemplary embodiments provided to enable persons skilled in the art to make or use the invention and not to limit the scope of the invention which is defined by the claims.

The define words and phrases below are a listing of some of the component parts or descriptions of elements that define the disclosed embodiments. The definitions are provided for the convenience of the reader and not to limit the scope of the invention which is defined by the claims.

“Adhesive” refers to a pressure sensitive, heat activated, two-part or other adhesive material for bonding one component to another.

“Aligned polarized conjugate” refers to electrically aligned polarized capture molecule conjugate.

“Antenna” refers to wireless communications signal transmitter and receiver.

“Applied field responsive end” refers to an end of a molecule that is responsive to an applied electrical or magnetic field.

“Back gate” refers to the back gate of a semiconductor biosensor.

“Biosensor” refers to a testing unit for sensing the presence of a pre-determined biomarker.

“Biosensor on water permeable substrate” refers to a biosensor provided on a water permeable substrate for selectively drawing away water from an EBC sample.

“Bottom driving electrode” refers to a lower driving electrode.

“BTS” refers to Biomarker Testing System.

“Buses” refers to signal communication structures.

“Capture molecule” refers to a ligand selected for its ability to bind to a target molecule.

“Capture molecule1” refers to a first capture molecule in a multi-capture molecule system.

“Capture molecule2” refers to a first capture molecule in a multi-capture molecule system.

“Capture pool” refers to a pool for collecting and holding EBC.

“Carrier fluid” refers to a fluid for containing and carrying a particulate or solute.

“Cellulose sponge” refers to compressed cellulose sponge.

“Condensate forming surface” refers to an actively chilled surface for condensing breath vapor into breath condensate.

“Control line” refers to an area of a lateral flow assay where excess labeled capture molecules aggregate to indicate a valid test.

“DAS module” refers to a data acquisition and storage module.

“Debye screening length” refers to an optimal distance from a transport surface for capture molecule binding with target molecule.

“Detection interface” refers to an area of a biosensor for detecting a target molecule.

“Detection well” refers to a well for receiving a holding a liquid biosample in contact with a detection area.

“Dissolvable adhesive” refers to a pressure sensitive or other adhesive and can be provided as a tape or coating.

“Dissolvable fluid dam” refers to a water soluble film for holding back the flow of EBC.

“Drain” refers to a semiconductor drain feature.

“Drainage hole” refers to a hole is a substrate for passing a portion of a liquid sample pad.

“Dry buffer/surfactant” refers to chemical additives that can be incorporated into the flow path of the EBC sample.

“EBC collector” refers to an actively cooled structure for converting exhaled breath vapor into exhaled breath condensate.

“EBC reservoir” refers to a reservoir for accumulating EBC.

“Electric field” refers to an applied electric field for aligning and/or attracting a field responsive end of a molecule.

“Electrode” refers to a conductive member for applying electrical energy.

“Electronics” refers to a printed circuit board having elements such as a Processor and communications circuit for determining and communicating a test result.

“Endothermic materials” refers to chemical components that when mixed together absorb heat from the surroundings.

“Epoxy base” refers to a base of a packaged semiconductor device.

“Epoxy top” refers to a top cover of a package semiconductor device.

“Exhaled breath condensate” refers to a liquid sample obtained by cooling breath vapor to form liquid droplets.

“Exhaled breath vapor” refers to the relatively warmer moisture carrying breath that exits the lungs through the mouth and nose, and that cools on the relatively warmer actively chilled condensation surface.

“Face mask” refers to a covering for the face that creates a containment volume for holding exhaled breath vapor in contact with the actively chilled condensation surface.

“Fluid transfer” refers to a material or structure for transporting a liquid sample.

“Fragment” refers to a piece of a virus or other organism that can be detected as a biomarker.

“Free-floating molecule” refers to a polar conjugate molecule that is free floating in a carrier fluid.

“Ganged biosensor” refers to two or more biosensors electrically sharing electronics for detecting one or more biomarkers.

“Gate” refers to a gate features of a semiconductor biosensor.

“Gold nano-particle” refers to a nano-sized gold particle.

“Graphene” refers to a charge transport material disposed at the detection interface.

“Heating element” refers to a small heat source for increasing the temperature for improving molecular binding.

“Hydrophilic structure” refers to a surface or structure that is easily wet by water.

“Hydrophobic surface” refers to a surface that is not easily wet by water.

“Label” refers to a molecule tagged to a capture molecule.

“Linker” refers to a molecule that binds to a charge transport layer and a capture molecule.

“Liquid gate electrode” refers to an electrode for applying voltage to a liquid sample to form a liquid gate for a semiconductor biosensor.

“Lysing material” refers to a chemical or mechanical mechanism for opening a virus particle to release biomarkers.

“Magnetic field” refers to a magnetic field applied as an attractive and/or aligning field.

“Magnetic nano-particle” refers to a small sized particle of magnetic material.

“Magnetic trap” refers to a permanent or electronically controlled magnetic source for holding magnetic nano-particles.

“Membrane” refers to a sample flow membrane of a lateral flow assay.

“Memory” refers to computer information storage.

“MME/SGW” refers to a mobility management entity/serving gateway.

“N-protein” refers to a biomarker protein of a virus, such as SARS-CoV-2.

“NanoCLAMP” refers to a small sized capture molecule particularly suited for use in a g-FET biosensor.

“NFC antenna” refers to a near field communication antenna and signal conditioning circuit.

“Node” refers to a communications network element.

“Non-solvent carrier fluid” refers to a fluid that contains a capture molecule and field orientable nano-particle, and that is a non-solvent for the dissolvable adhesive.

“Non-target protein” refers to a superfluous protein in an EBC sample.

“Patterned teflon sheet” refers to teflon sheet that is laser, calendared or sandblasted.

“Polarizable end” refers to a portion of a molecule or conjugate that becomes more polar upon binding with a target molecule.

“Pooling area” refers to a structure for aggregating EBC droplets.

“Processor” refers to a microprocessor or microcontroller.

“RRH” refers to a remote radio head.

“S-protein” refers to a biomarker protein of a virus such s SARS-CoV-2.

“Sample pad” refers to a sample receiving portion of a lateral flow assay.

“SAP” refers to Super Absorbent Polymer.

“SAP bead” refers to a super absorbent polymer bead.

“Selectively permeable membrane” refers to a membrane that allows one material to pass through it while blocking the passage of another material.

“Separator” refers to a member for separating lower and higher target molecule concentration wicks.

“Source” refers to a semiconductor source feature.

“Substrate” refers to a sheet member for supporting a testing system.

“Superstrate” refers to a sheet member on which is formed or supported a biosensor or test area.

“Surfactant” refers to a chemical for disrupting lipid structures.

“Target molecule” refers to a biomarker or other chemical selected for detection in a sample.

“Target molecule-labeled capture molecule complex” refers to a labeled capture molecule that is bound to a target molecule.

“Target protein” refers to a target molecule protein contained in an EBC sample.

“TBCA Module” refers to a Target Biomarker Collection and Analysis module.

“Teflon spikes” refers to a texture teflon or other plastic or metal surface with spike protrusions for affecting the flow or a liquid biosample and/or for disrupting the shell or cell wall of a microbe.

“Test line” refers to an area of a lateral flow assay where labeled capture molecule and target molecule conjugate accumulate to indicate the detection of the target molecule.

“Testing area” refers to a location of detection of a target molecule in a sample.

“Testing system support” refers to a support structure for holding the EBC collector and other components on the inside of a face mask.

“Thermal mass” refers to a structure that can be suitably used to absorb heat from a vapor to condense the vapor into a liquid.

“Time release lysing beads” refers to dry lysing material that can be provided in the fluid path for lysing microbes to release biomarkers.

“Top driving electrode” refers to an upper driving electrode.

“Transceiver” refers to transmitter and/or receiver electronic circuitry.

“Urea crystals” refers to an endothermic chemical component that when mixed with water removes heat from the surroundings.

“Virus” refers to an example of an infectious disease transmitting organism.

“Virus-concentrated EBC” refers to an EBC sample that has a portion of water removed to concentrate the virus present in the remaining EBC collector.

“Water” refers to a water drop or pool.

“Water bag” refers to a vessel, pouch, or space between adhesive sheets, that contains water or other liquid endothermic material or thermal mass.

“Wick” refers to an absorbent material for receiving a fluid sample.

“Wireless link” refers to a wireless communication link.

“Z-axis conductive tape” refers to an adhesive tape with z-axis conductors for conducting an electrical signal in the z-axis direction only.

Many configurations, embodiments, methods of manufacture, algorithms, electronic circuits, microprocessors, memory and computer software product combinations, networking strategies, database structures and uses, and other aspects are disclosed herein for a diagnostic or testing platform, devices, methods and systems that have a number of medical and non-medical uses.

Although embodiments are described herein for detection of biomarkers of SARS-CoV-2 virus, the systems, methods and apparatus described are not limited to any particular virus or disease, or just limited to biological use-cases. In most instances, where the term virus or COVID-19 is used, any other health or fitness related biomarker could be used instead. The description here and the drawings and claims are therefore not intended to be limited in any way to virus detection, the inventions described and claimed can be used for many diseases including lung cancer, diabetes, asthma, tuberculosis, environmental exposures, glucose, lactate, blood borne diseases and other ailments or indications of the health of the test subject. Further, the electronic biosensor, test systems, uses and methods of manufacturing described herein are not limited to the use of exhaled breath condensate. Wastewater, potable water, environmental quality samples, ambient samples and any bodily fluid can be used as the test sample. The use of aptamers and engineered capture molecules, in particular, make the inventive sensor widely useful because of the nature of selected aptamers being adaptable by specific engineering design and selection to have a binding affinity that is tailored to a corresponding target analyte. Therefore, the descriptions of innovations are not intended to be limited to a particular use-case, capture molecule, biomarker or analyte.

In immunochromatography, a capture molecule, which may be, for example, an aptamer, naturally occurring antibody, or engineered antibody, is disposed onto a surface of a porous membrane, and a sample passes along the membrane. As described herein, the term antibody, aptamer, engineered antibody, or capture molecule is used interchangeably. In some instances, a specific type of capture molecule may be described. In the case of a lateral flow assay (LFA) type testing system, biomarkers in the sample is bound by the capture molecule which is coupled to a detector reagent. As the sample passes through the area where the capture molecule is disposed, a biomarker detector reagent complex is trapped, and a color develops that is proportional to the concentration or amount biomarker present in the sample. In the case of an electronic or electro-chemical testing system, the captured biomarker causes a detectable change in a signal obtained, typically through an electrical connection with two or more electrodes.

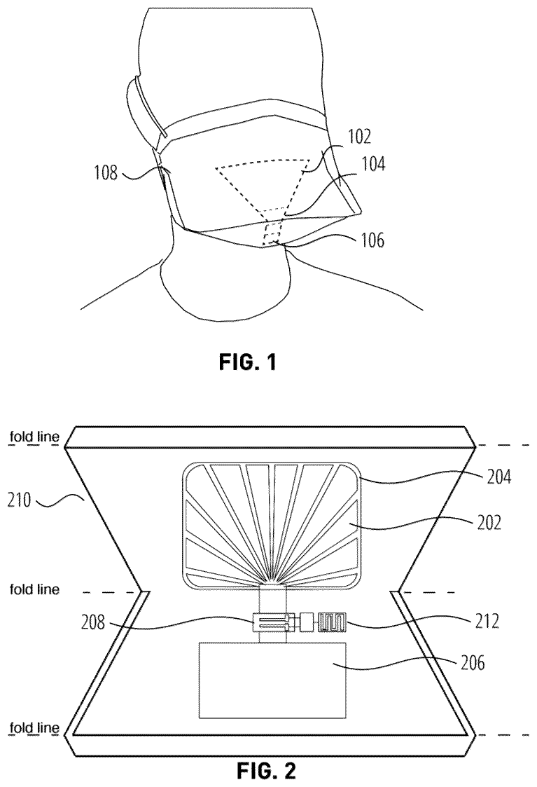

FIG. 1 shows a system including an EBC collector 102, a fluid transfer 104, an NFC antenna 106, and a face mask 108.

A face mask and a mask-based diagnostic platform is shown with an Exhaled Breath Condensate (EBC) collector, fluid transfer system, biosensor testing system and near field communication (NFC) antenna and signal condition circuit all disposed within the confined volume of the inside of the face mask. The biosensor shown is an electrical or electrochemical biosensor, such as a g-FET or other electronic biosensor construction. Alternatively, the exemplary embodiments disclosed herein can be used with other testing system, such as LFAs, cellulose-based biosensors, color change reagent solutions, and the like. In some cases, the EBC collector and face mask are used to collect a fluid biosample which is tested outside the mask on a desktop or laboratory testing system (such as conventional PCR testing), in other cases, the testing systems are incorporated directly into and/or onto the face mask as a self-contained testing apparatus. In most cases, the requirements of the test subject are simply to put the face mask on and breathe normally while the biosample is obtained, and typically there is no need for a technician or other trained personnel for the test or sample collection to be completed.

In accordance with a non-limiting embodiment, a mask-based diagnostic platform is provided for detecting a biomarker received from lungs and airways of a test subject. An EBC collector is disposed on an inside of a face mask worn by the test subject. The EBC collector converts breath vapor received from the lungs and airways of the test subject into a fluid biosample. The EBC collector has a thermal mass or other cooling system, such as a frozen water/super absorbent polymer gel, a chilled metal plate or an endothermic reaction (described herein) that cools the condensing surface that receives the breath vapor at a temperature greater than a surface temperature of the condensing surface. The breath vapor is coalesced into liquid droplets on the condensing surface. The EBC collector may include a droplet harvesting structure including a field for receiving the breath vapor and forming fluid droplets, and channels for receiving the fluid droplets from the field and channeling the fluid droplets in the form of a collected fluid biosample to a fluid transfer system, such as microfluidic, capillary or other fluid conducting structure.

A biosensor is fixed to the face mask for receiving the fluid biosample from the EBC collector and testing the fluid biosample for a target biomarker. The biosensor generates a test signal dependent on at least the presence and absence of the target biomarker in the fluid biosample. A signal condition circuit, such as an amplifier, can be provided if needed to receive the test signal that is transmitted via a near field communication (NFC) antenna to a wireless receiver, such as a cellphone. Also, the signal conditioning circuit may include energy harvesting electronics that receive radio frequency energy, for example, transmitted from the cellphone and received by the NFC antenna. The energy harvesting antenna may include, for an example, a capacitor or other circuit elements so that the biosensor is operated using the harvested energy with no need for an onboard battery or other energy source.

FIG. 2 shows a system comprising an NFC antenna 106, a face mask 108, a hydrophobic surface 202, a hydrophilic structure 204, a wick 206, and a biosensor 208.

The inside of the mask-based diagnostic platform is shown splayed open with components for collecting and testing EBC. The EBC collector and testing system can be retrofitted into an existing mask or integrated into the formation of a mask. FIG. 2 shows a simple, low cost, disposable mask construction. The mask base material can be multi-stack, N95-type mask material, filter material, cloth or paper, or a breathable non-woven polymer material with micropores that allow air exchange. The EBC collector with hydrophobic fields and hydrophilic channels is fixed on the mask material. The fluid sample collected by the EBC collector is transferred by microfluidic transfer materials to the biosensor and, as described herein, can be allowed to pool on the biosensor or flow over the biosensor using a wicking material located downstream from the biosensor testing area. The biosensor testing area is small, typically a few millimeters squared or less in surface area, although a larger area and multiple biosensors or testing zones can be provided. The biosensor device has electrodes with leads that enable electrical communication with the signal conditioning circuit and NFC antenna.

This configuration creates a low cost biosample testing and communications system that can be used, along with an APP running on a cellphone to determine from the test signal a wirelessly transmittable test result depending on detecting or not detecting the target biomarker. The test result can be transmitted from the cellphone to a remote receiver, such as a cloud-based or local server. Other wired or wireless communication systems can be used. In the case of a relatively more expensive test reader and communication electronics, preferably, the electronics and battery are disposed on the outside of the mask when in use, and the EBC collector, microfluidic transfer materials, biosensor and wicking materials are disposed on the inside of the mask. After use, the electronics can be removed from the outside of the mask and sanitized for a next use. The disposable mask and components located inside the mask (and exposed to the most potential contamination) can be sealed in a suitable bag and thrown away.

To capture aerosol droplets and particulate, optionally, a dissolvable adhesive patch can also be provided on the inside surface of the mask. The mask-based diagnostic platform can be provided with this particulate capturing structure for receiving and capturing exhaled breath aerosol (EBA) particulate from airway linings of a user. The particulate capturing structure includes a dissolvable EBA sample collector film for capturing EBA particulate. In this case, for example, a forced cough while wearing the mask ejects aerosol droplets and particulate from the lungs and airways that get captured on the dissolvable adhesive patch. The fluid biosample testing components described herein can be used to provide a rapid screening test, and if a positive infection is detected, the mask can be placed into a hermetically sealed envelope and brought to a lab for more rigorous testing of the fluid biosample, aerosol droplets and particulate held by the mask and sealed in the envelope. Also, a vessel for holding the fluid biosample may be provided, or the pooling area can include a pressure sensitive adhesive strip for sealing in the collected EBC for transport to a testing lab.

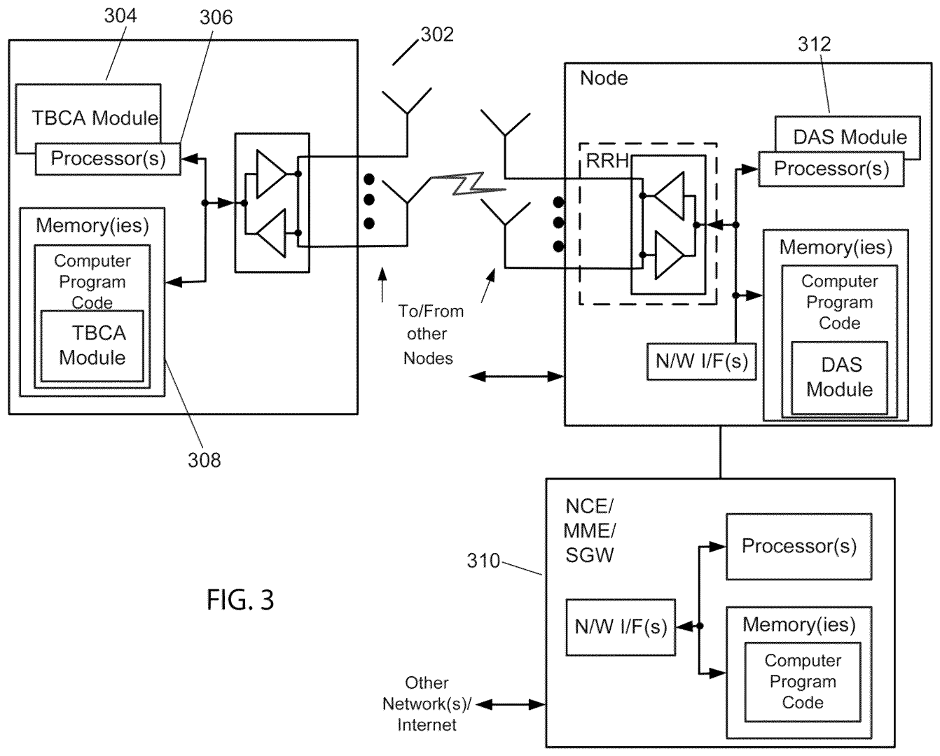

FIG. 3 shows a system comprising an antenna 302, a TBCA Module 304, a Processor 306, a Memory 308, an MME/SGW 310, and a DAS module 312.

A block diagram is shown of one possible and non-limiting exemplary system in which the exemplary embodiments may be practiced where the installed wireless communications networks and Internet can be utilized for an ultra-large scale deployment of testing for a target biomarker, such as a virus spreading through a population. Co-ordinated testing and data collection of a population can be accomplished throughout a city, state, country or even world-wide. For example, the interconnected cellular, LANs, WANs and Internet can be employed to quickly obtain important data indicating the presence of a pathogen, such an endemic or pandemic virus, at an airport, in a small community, a large city or a larger population. The test result data from many diagnostic tests can be automatically and conveniently collected and aggregated into large data sets for Big Data analysis by Machine Learning and Artificial Intelligence agents, enabling rapid pattern recognition and predictive models to be generated to indicate to governments, hospital administrators, NGOs, and other authorities where an outbreak of a virus is occurring, how rapidly the outbreak is spreading, etc. In this exemplary embodiment, the communications infrastructure of a conventional cellular communication system is utilized to quickly obtain test results from a large number of test subjects and transmit the test results in at least two data streams: 1) with patient identifying information to a trusted receiver (e.g., a patient's doctor so that the individual patient can be appropriately monitored and treated), and 2) without patient identifying information (for patient privacy purposes) for aggregation and Big Data population analysis.

A biomarker testing system (BTS) is in wireless communication with a wireless network. A BTS is a wireless biomarker testing system that can access a wireless network, such as the communications-enabled mask-based diagnostic platform described herein. The BTS includes one or more Processors, one or more Memories, and one or more Transceivers interconnected through one or more Buses 127. Each of the one or more Transceivers includes a receiver, Rx, and a transmitter, Tx. For some use-cases, there may be no need for an onboard receiver in the mask-based diagnostic unit. The one or more Buses may be address, data, or control Buses, and may include any interconnection mechanism, such as a series of lines on a motherboard or integrated circuit, or other communication equipment, and the like. The one or more Transceivers are connected to one or more antennas. The one or more Memories include computer program code. The BTS 110 includes a Target Biomarker Collection and Analysis (TBCA) module, comprising the inventive biosensor testing system described herein. An embodiment of the TBCA also includes wireless communication capabilities comprising one of or both parts, which may be implemented in a number of ways. The TBCA Module may be implemented in hardware as TBCA Module such as being implemented as part of a mask-based diagnostic system that includes the one or more Processors. The Processors of the TBCA Module may be implemented also as an integrated circuit or through other hardware such as a programmable gate array. In another example, the TBCA Module may be implemented as TBCA Module, which is implemented as computer program code and is executed by the one or more Processors, where test results are obtained in the form of analog or digital electrical signals generated by a biosensor used to test a sample, such as an EBC sample. For instance, the one or more Memories and the computer program code may be configured to, with the one or more Processors, cause the biomarker testing system to perform one or more of the operations as described herein. The BTS communicates with Node via a wireless link.

In an exemplary cellular communication model, a near field communication system is used to obtain test results from the biosensor of a mask-based diagnostic system. The NFC system enables convenient and low-cost obtainment of the test result using a conventional hand-held cellular telephone, computer, communications pad, or a dedicated wireless communication reader/transmitter/receiver.

The test results are transmitted to the Node, which is typically a base station of a wireless communications network (e.g., 5G, 4G, LTE, long term evolution or any other cellular, internet and/or wireless network communication system) that provides access by wireless devices such as a cellular telephone BTS to the wireless network. For example, at a stadium or airport, the wireless communication infrastructure of the venue can be utilized for direct communication with the mask-based diagnostic system, and/or the user's cellphone can be used as a relay.

The Node includes one or more Processors, one or more Memories, one or more network interfaces (N/W I/F(s)), and one or more Transceivers interconnected through one or more Buses. Each of the one or more Transceivers includes a receiver, Rx and a transmitter, Tx. The one or more Transceivers are connected to one or more antennas. The one or more Memories include computer program code. The Node includes a Data Acquisition and Storage (DAS) module, comprising one of or more parts, which may be implemented in a number of ways. The DAS module may be implemented in hardware as DAS module such as being implemented as part of the one or more Processors. The DAS module may be implemented also as an integrated circuit or through other hardware such as a programmable gate array. In another example, the DAS module may be implemented as DAS module which is implemented as computer program code and is executed by the one or more Processors. For instance, the one or more Memories and the computer program code are configured to, with the one or more Processors, cause the Node to perform one or more of the operations as described herein. The one or more network interfaces 161 communicate over a network such as via the links. Two or more Nodes communicate using, e.g., link. The link may be wired or wireless or both and may implement, e.g., an X2 interface.

The one or more Buses may be address, data, or control Buses, and may include any interconnection mechanism, such as a series of lines on a motherboard or integrated circuit, fiber optics or other optical communication equipment, wireless channels, and the like. For example, the one or more Transceivers may be implemented as a remote radio head (RRH), with the other elements of the Node being physically in a different location from the RRH, and the one or more Buses could be implemented in part as fiber optic cable to connect the other elements of the Node to the RRH.

The wireless network 100 may include a network control element (NCE) that may include MME (Mobility Management Entity)/SGW (Serving Gateway) functionality, and which provides connectivity with a further network, such as a telephone network and/or a data communications network (e.g., the Internet). The Node is coupled via a link to the NCE. The link may be implemented as, e.g., an S1 interface. The NCE includes one or more Processors, one or more Memories, and one or more network interfaces (N/W I/F(s)), interconnected through one or more Buses. The one or more Memories include computer program code. The one or more Memories and the computer program code are configured to, with the one or more Processors, cause the NCE to perform one or more operations.

The wireless network 100 may implement network virtualization, which is the process of combining hardware and software network resources and network functionality into a single, software-based administrative entity, a virtual network. Network virtualization involves platform virtualization, often combined with resource virtualization. Network virtualization is categorized as either external, combining many networks, or parts of networks, into a virtual unit, or internal, providing network-like functionality to software containers on a single system. Note that the virtualized entities that result from the network virtualization are still implemented, at some level, using hardware such as Processors and Memories, and also such virtualized entities create technical effects.

The computer readable Memories may be of any type suitable to the local technical environment and may be implemented using any suitable data storage technology, such as semiconductor-based Memory devices, flash Memory, magnetic Memory devices and systems, optical Memory devices and systems, fixed Memory and removable Memory. The computer readable Memories may be means for performing storage functions. The Processors may be of any type suitable to the local technical environment, and may include one or more of general purpose computers, special purpose computers, microprocessors, digital signal Processors (DSPs) and Processors based on a multi-core Processor architecture, as non-limiting examples. The Processors may be means for performing functions, such as controlling the C19TS, Node, and other functions as described herein.

In general, the various embodiments of the biomarker testing system 110 can include, but are not limited to, wireless communication components used for Bluetooth, cellular telephones such as smart phones, tablets, personal digital assistants (PDAs) having wireless communication capabilities, portable computers having wireless communication capabilities, image capture devices such as digital cameras having wireless communication capabilities, gaming devices having wireless communication capabilities, music storage and playback appliances having wireless communication capabilities, Internet appliances permitting wireless Internet access and browsing, tablets with wireless communication capabilities, as well as portable units or terminals that incorporate combinations of such functions.

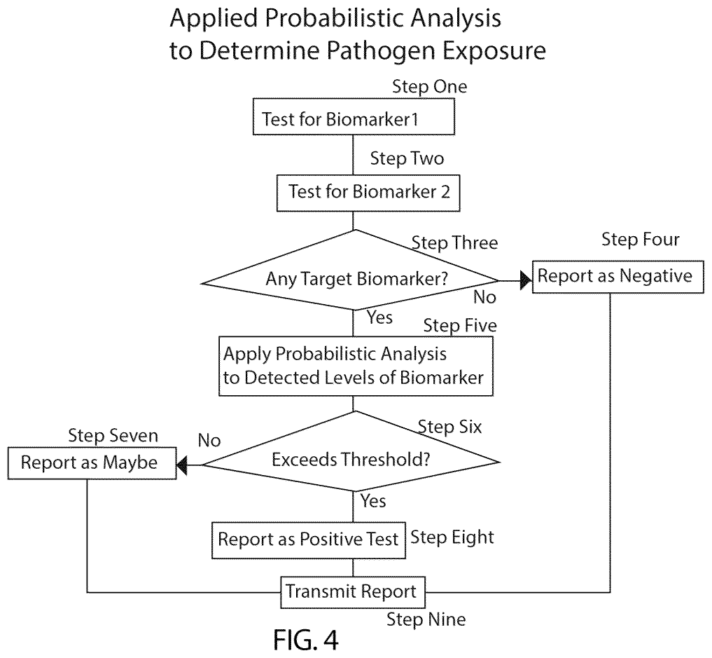

FIG. 4 is a logic flow diagram for Applied Probabilistic Analysis to determine the detection of a target biomarker, and illustrates the operation of an exemplary method, a result of execution of computer program instructions embodied on a computer readable Memory, functions performed by logic implemented in hardware, and/or interconnected means for performing functions in accordance with exemplary embodiments. For instance, the TBCA Module 140 may include multiples ones of circuit elements for implementing the functions shown in the blocks in FIG. 3, where each included block is an interconnected means for performing the function in the block. At least some of the blocks in FIG. 3 are assumed to be performed by the BTS e.g., under control of the TBCA Module at least in part.

Currently, the world is dealing with a pandemic caused by a novel coronavirus, SARS-CoV-2. This virus pathogen has caused millions of deaths, the shutting down of the economics of many nations, and trillions of dollars of economic loss world-wide. To combat the spread of the virus, billions of people around the world have halted their usual employment, entertainment and socializing activities. Testing for biomarkers that indicate exposure, infection and recovery from the virus pathogen can be used to enable a safer and more efficient restart of economic activities, while minimizing the spread of the virus, and keep a watch out for the progression of variants of the virus within a community, nation or world-wide.

For example, protein and RNA testing for active virus shows who is currently contagious. Antibody testing can be used to find the members of a population that have recovered from the virus and now may be immune to reinfection. During this current and a future pandemic, this knowledge can enable precision social distancing and more effective contact tracing, with the re-employment of a growing workforce of protected individuals and consumers. Those who remain at-risk of infection and transmission can be kept sequestered until a vaccine or other solution such as a high success rate pharmaceutical therapy is developed and made widely available. In the future, a rapid deployment of testing system, such as the mask-based diagnostic platform described herein, especially when the wireless communications infrastructure is used as described in FIGS. 3-6 and elsewhere herein, can quickly ascertain the location and speed of a pathogen spread through a population and identify those who are infected prior to traveling and bringing the virus to uninfected parts of the world.

The COVID-19 pandemic is an example of a viral pathogen that has required every nation to deploy massive resources in combating the disease, and the shutting down of economies, in an attempt to limit the spread of the virus while balancing the economic and social impacts on the population. Testing the population for the virus, tracking the pandemic spread and estimating the size of the infected population is a crucial tool to combat the current and any future endemic or pandemic outbreak.

Syndromic testing simultaneously tests for multiple pathogens with overlapping symptoms. In accordance with an exemplary embodiment, multiple biosensors are used to enable the simultaneous detection of a number of biomarkers, increasing the accuracy of test.

Stochastic analysis of data is used to handle changes that involve both randomness and uncertainty, aspects that are particularly difficult to manage during a fast spreading virus outbreak. More specifically, partially-observable stochastic analysis can account for incomplete knowledge of a contagious pathogen spreading through a population. For example, partial observation provides insights but only with a certain degree of certainty. In the case of a biological invasion or virus spread, testing for infection can often result in a false negative, so that the infection can then only be detected within a certain probability for an infected individual.

Applied probabilistic analysis can be used to improve the predictive model of an individual's infection status and in the aggregate, help to refine the testing results thresholds for an objective quantitative or qualitative testing system. In accordance with an exemplary embodiment, for the applied probabilistic analysis to determine pathogen exposure, Biomarker1 is first tested for (step one), Biomarker2 is then tested for (step two). Additionally, BiomarkerN is tested for where N can be any number of multiple biomarkers tested using the inventive testing system. If no target biomarker is detected (step three) then a Negative Test report is generated (step four). If any target biomarker is detected (step three) then probabilistic analysis may be performed depending simply on the detected presence (yes/no) or quantitative analysis (e.g., concentration) of the one or more detected biomarkers (step five). The probabilistic analysis can be performed using an updated probability model where probabilistic multipliers for the tested—for biomarkers are determined for a population. As an example, if a virus outbreak occurs earlier in time in a region or country different from the location of the currently applied testing, the probabilistic multipliers for the tested—for biomarkers can be determined from confirmed cases occurring during the earlier outbreak. A threshold can be determined for the results of the probabilistic analysis based on the probabilistic multipliers obtained from the confirmed cases, and help to improve the accuracy of the testing system. As an example, in an electronic biosensor, a threshold voltage for considering a test result as positive can be adjusted based on the probabilistic analysis of previously tested and confirmed positive cases. Over time, the accuracy and confidence of positive and negative determinations is improved based on the history of confirmed cases and obtained threshold voltages. As the database of tested cases grows, the overall testing regimen with interconnected communication, sharing and analysis of tests results is used to automatically improve the accuracy and confidence of future tests.

For examples of probabilistic analysis used for modeling the SARS-CoV-2 pandemic, see, Modeling the dynamics of the COVID-19 population in Australia: A probabilistic analysis, Eshragh et al., Oct. 2, 2020, https://doi.org/10.1371/journal.pone.0240153; Estimated Incidence of Coronavirus Disease 2019 (COVID-19) Illness and Hospitalization—United States, February-September 2020 Reese, et al., CID 2021:72, June 2021; and Güemes, A., Ray, S., Aboumerhi, K. et al. A syndromic surveillance tool to detect anomalous clusters of COVID-19 symptoms in the United States. Sci Rep 11, 4660 (2021). https://doi.org/10.1038/s41598-021-84145-5, the disclosures of which are all incorporated herein in their entireties.

If the probabilistic analysis does not exceed a threshold (step six) (e.g., low concentration of a particular target biomarker, or the presence of just one weak biomarker indicating likely infection), then a Maybe Test report is generated (step seven). If the probabilistic analysis does exceed a threshold (step six) (e.g., high concentration of a particular target biomarker, or the presence of two or more biomarkers indicating likely infection), then a Positive Test report is generated (step eight). The Test Report is then transmitted (step nine) (e.g., in a manner described herein or other suitable transmission mechanism including verbal, digital, written or other communication transmission that adds to the accumulated database of test results).

The logic flow of FIG. 4 is implemented by a non-limiting embodiment of an apparatus, comprising at least one Processor; and at least one Memory including computer program code, the at least one Memory and the computer program code configured to, with the at least one Processor, cause the apparatus to perform at least the following: detecting one or more biometric parameters using a droplet harvesting structure for converting breath vapor to a fluid droplet for forming a fluid sample and a testing system having a biomarker testing zone for receiving the fluid sample and detecting the biometric parameter, where the biometric parameters are biomarkers dependent on at least one physiological change to a patient in response to a concerning condition such as a virus infection; receiving the one or more biometric parameters and applying probabilistic analysis to determine if at least one physiological change threshold has been exceeded dependent on the probabilistic analysis of the one or more biometric parameters; and activating an action depending on the determined exceeded said at least one physiological change.

In accordance with an embodiment, a biosensor testing device is provided having one or more biometric detectors each for detecting biomarkers as one or more biometric parameters. The biometric parameters are dependent on at least one physiological change to a patient or test subject, such as the production of immune response chemicals, the presence in the body of an active or deactivated virus or virus component, antibodies, antigens, virus RNA or DNA, or other biomarker inducing change (including an immune response or viral load count). A microprocessor receives the one or more biometric parameters and determines if at least one physiological change threshold has been exceeded depending on the one or more biometric parameters. An activation circuit activates an action depending on the determined physiological change. The action includes at least one of transmitting an alert, modifying a therapeutic treatment, and transmitting data dependent on at least one physiological change, the one or more biometric parameters, and therapeutic treatment.

The inventive mask-based diagnostic platform, and/or components of the platform described herein, can also be used to monitor the progression of a disease in a patient, for example, a hospitalized patient that is going through the disease progression of Covid-19. The at least one physiological change can also be in response to an applied therapeutic treatment that causes a change in the condition of the patient to enable the monitoring of the body's response to an applied therapeutic. The action can include transmitting an alert, modifying a therapeutic treatment, and transmitting data dependent on at least one of the at least one physiological change, the one or more biometric parameters, and therapeutic treatment. The microprocessor can analyze the one or more biometric parameters using probabilistic analysis comprising determining from a data set of the one or more biometric parameters whether the data set is acceptable for deciding that the at least one physiological change threshold has been exceeded. The probabilistic analysis can further comprise applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters.

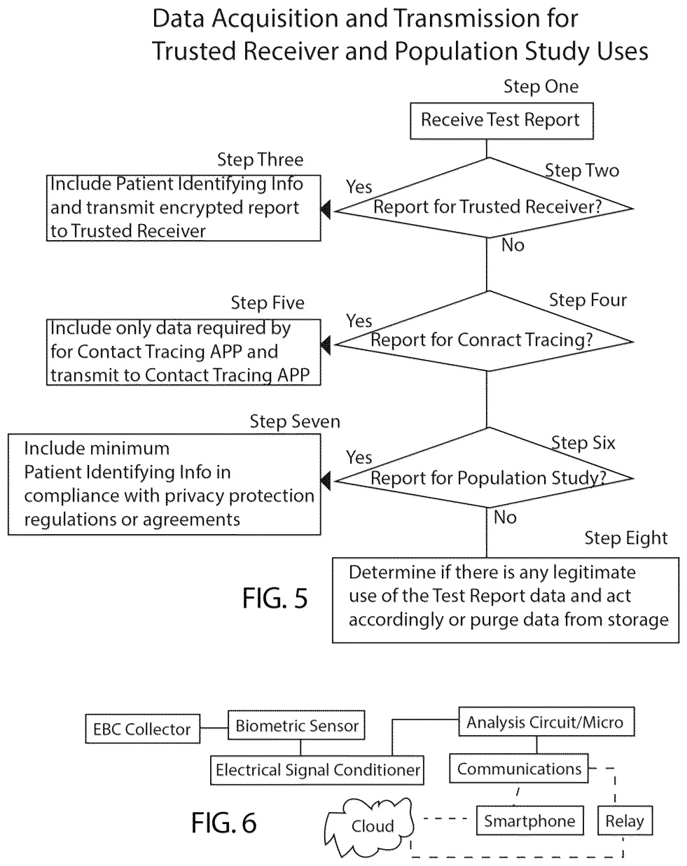

FIG. 5 is a logic flow diagram for Data Acquisition and Transmission for Trusted Receiver and Population Study uses, and illustrates the operation of an exemplary method, a result of execution of computer program instructions embodied on a computer readable Memory, functions performed by logic implemented in hardware, and/or interconnected means for performing functions in accordance with exemplary embodiments

The performance of the Data Acquisition and Transmission for Trusted Receiver and Population Study Uses process flow can be done at the testing system, Node, Smartphone, or combination of components located with the test subject or remote from the test subject, for example at storage location(s) of the acquired data. The acquired data can include patient or subject identifying information ranging from name, GPS location, list of known previous or future contacts (pre and/or post infection), prior medical history, demographics, etc. The Data Acquisition and Transmission for Trusted Receiver and Population Study Uses can be done at a secure server located anywhere on the network. For instance, the DAS module 150 may include multiple ones of circuit elements for implementing the functions shown in the blocks in FIG. 3, where each included block is an interconnected means for performing the function in the block. At least some of the blocks in FIG. 3 are assumed to be performed by a base station such as Node 170, e.g., under control of the DAS module 150 at least in part. A blockchain or other data security, storage and distribution technology can be used to enhance the privacy and controlled access to the de-identified patient data, while making this data available for researchers and authorities anywhere in the world.

The digital testing system architecture, manufacturing methods, and applications, can be used for capturing biometric data from the exhaled breath of a test subject or patient. Biometric data can be captured and transmitted continuously or at selected times with data access provided directly to a care-provider, enabling early diagnosis and ongoing monitoring, and to a researcher to gain valuable insights and assistance through AI analysis. This data detection is direct from the exhaled breath and can be provided through a wireless connection for Blockchain and AI database collection, access and analysis.

As shown in FIG. 5, a Test Report is received (step one) (e.g., from a Smartphone transmission from the patient or test subject). If the report is intended to be sent to a trusted receiver (step two), such as a patient's healthcare provider or insurance company, then an encrypted report can be generated (step three) and transmitted to the trusted receiver that includes patient identifying information. Two step verification or better, such as the verification protocols used to ensure online banking, can be used to make sure that the receiver of the patient's data is indeed a trusted receiver. If the report is not for a trusted receiver (step two) but instead is to be used for Contact Tracing (step four), then only the data required for Contact Tracking is transmitted. Also, for contact tracing, a Contact Tracing APP can be employed (step five). The Contact Tracing APP may be, for example, a system provided for identifying and notifying people who have come in contact with the test subject or patient within a given time prior or since testing for one or more target biomarkers. If the report is not for a trusted receiver (step two) or for contact tracing (step four) but instead is to be used for a Population Study (step six), then only the minimum patient identifying information in compliance with privacy regulations and/or agreements is transmitted and/or stored along with the received test report (step seven). If the report is not for a trusted receiver, contact tracing or population study (steps two, four, six) then it is determined if there is any legitimate use of the test report data and an action is taken accordingly or the data is automatically purged from storage (step eight).

FIG. 6 is a block diagram of the basic components for testing EBC and transmitting the test result to a smartphone and/or cloud server. The EBC collector provides a fluid sample that is received by the biometric detector or biosensor. An electrical signal conditioner, such as a signal amplifier, filter, etc. can be provided to condition the raw test signal from the biosensor before a microprocessor or analysis circuit determines the test result signal. After processing the conditioned signal, a test result signal is transmitted via a communications circuit, which may be the NFC system described earlier, or Bluetooth or other wired or wireless communications method and structures. A smartphone or access point relay can be used to receive the wireless test result signal and transmit it to the cloud.

In accordance with an embodiment, the electronic circuit comprises an amplification circuit for receiving the test signal from the biosensor and amplifying the test signal to an amplified electrical signal. A comparator circuit compares the amplified electrical signal with a pre-determined value based on at least one of a computer model-derived and empirically-derived electrical signal calibration of the biosensor. The calibration can be determined using at least one of a known presence and a known concentration of the target analyte in a calibration sample. The comparator circuit generates the test result signal based on the amplified electrical signal compared with the pre-determined value.

The electronic circuit can also comprise an analyte concentration circuit for determining a concentration value of the target analyte depending on the amplified electrical signal. In this case, the amplified electrical signal changes value depending on a number of target analyte molecules in the fluid biosample, and the test result signal is dependent on the determined concentration value.

In accordance with an embodiment, the electronic circuit further comprises a wireless communication circuit for wirelessly transmitting the test result signal to at least one of a smart phone, tablet, computer, relay, access point and computer network.

FIG. 7 shows a system comprising a face mask 702, an EBC collector 704, a condensate forming surface 706, a fluid transfer 708, and a biosensor 710. A KN95 face mask is shown with a prototype retrofittable testing system including EBC collector having an aluminum foil condensate-forming surface, embossed fluid conductor channels, a microfluidic fluid transfer system and electronic biosensor electrodes.

FIG. 8 shows a system comprising a face mask 802 and an EBC collector 804, showing the prototype testing system retrofit into the KN95 mask.

FIG. 9 shows a system comprising an electronics 902 and a face mask 904, showing the KN95 mask having removable testing and communication electronics disposed on the outside of the mask

FIG. 10 shows a system comprising a testing system support 1002, a biosensor 1004, an EBC collector 1006, a hydrophilic structure 1008, and a hydrophobic surface 1010. An Exhaled Breath Condensate Collector and a testing area is shown with ganged syndromic testing biosensors. In accordance with an exemplary prototype embodiment, a mask-based EBC collector is fabricated from 30 cm wide rolls of 120 μm thick natural virgin Polytetrafluoroethylene (PTFE) sheet (eplastics.com., USA), 10 cm wide 3M 465 double sided adhesive transfer tape (uline.com, USA) as well as a super absorbent polymer (SAP) powder, MediSAP 715 (M2 Polymer Technologies, Inc., Illinois, USA). A stamping jig can be constructed, for example, from 0.315 cm PTFE plate (eplastics.com, USA) and cut on a 100 W CO2 laser cutter (Orion Motor Tech, China). A Digital Combo Heat Press (Geo Knight, Massachusetts, USA) can be used to stamp the 127 μm thick PTFE sheet using the jig to form a pocket in the PTFE sheet for receiving a thermal mass mixture of water and the SAP. A second layer of the 127 μm PTFE sheet may be bonded to the heat stamped 127 μm PTFE sheet using a 3M 465 adhesive, sandwiching the thermal mass of water/SAP between layers of PTFE sheet. The completed PTFE/thermal mass/3M 465/PTFE laminated sandwich can be cut using a laser or die cutter into the final shape of the EBC collector that is configured and dimensioned to be inserted into a pre-existing face mask or built into a newly constructed face mask. The EBC collector constructed as described may be retro fit into various disposable face masks of different styles and constructions, including N95 and KN95 made by 3M and other manufacturers.

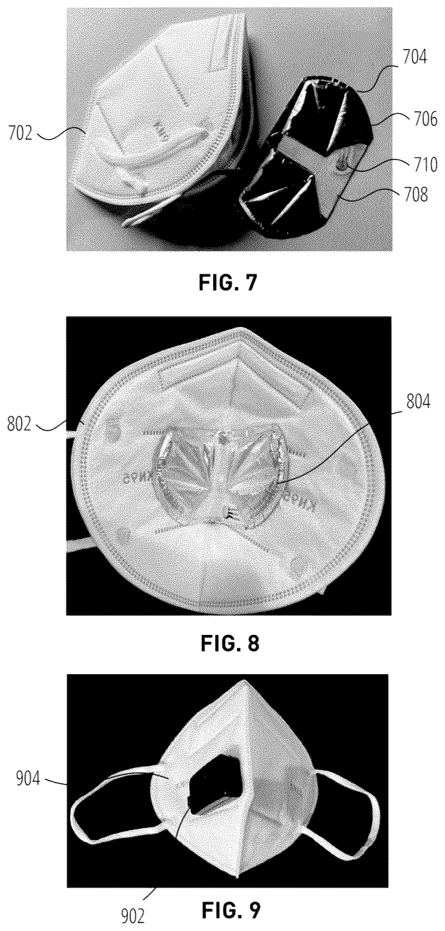

FIG. 11 shows a system comprising a pooling area 1102 and an adhesive 1104, showing the pooling area that forms a volume for holding the biosensors immersed in collected EBC.

FIG. 12 shows a system comprising an EBC collector 1202, a testing system support 1204, and a pooling area 1206. FIG. 12 shows an assembled EBC collector and pooling area. An exhaled breath condensate (EBC) collector converts breath vapor received from the lungs and airways of the test subject into a fluid biosample. The EBC collector includes a condensate-forming surface, and a thermal mass in thermal connection with the condensate-forming surface. A fluid transfer system transfers the EBC to at least one of a testing unit and an EBC containment vessel. The testing unit may be one or more electronic biosensors, one or more lateral flow assays or other microfluidic type testing systems, or a combination of the same. A testing system support for the EBC collector is configured and dimensioned to fit inside a face mask. The face mask forms an exhaled breath vapor containment volume to hold the exhaled breath vapor in proximity to the EBC collector to enable the exhaled breath vapor to efficiently coalesce into the fluid biosample.

As described in more detail herein, the testing unit may comprise a g-FET biosensor having a detection interface comprising a graphene layer functionalized with capture molecules, wherein the capture molecules are smaller than the Debye screening length. As an example of a syndromic testing device, the biosensors can be designed to bind to FluA, FluB, SARS N-protein (more conserved, slower to mutate protein across SARS viruses) and S-protein (faster to mutate, cause of the SARS-CoV-2 variants).

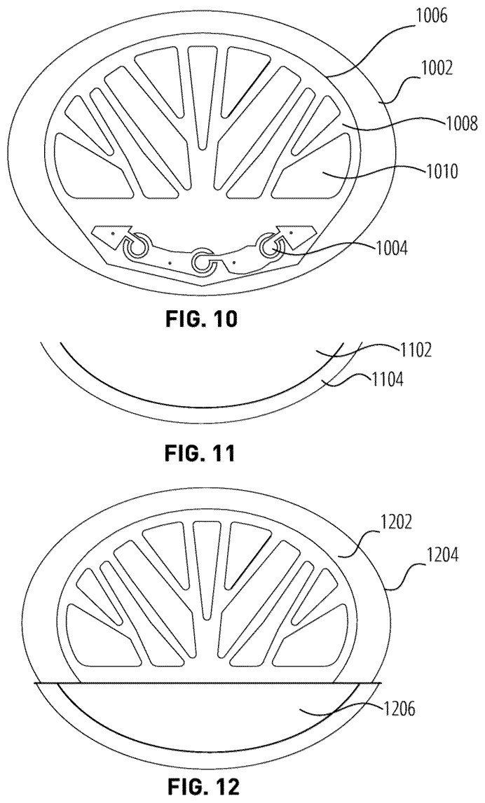

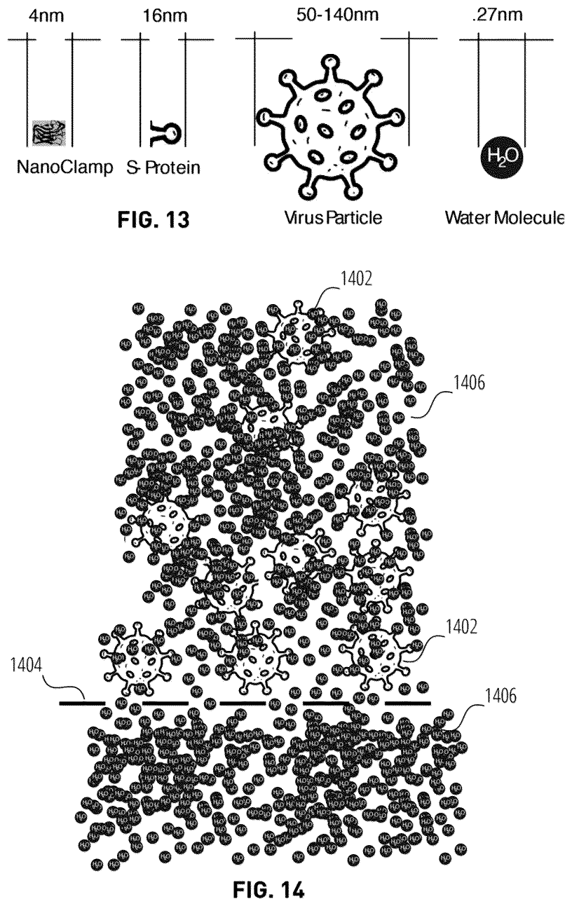

FIG. 13 shows the relative sizes of an engineered capture molecule known as a nanoCLAMP, an S-protein, a virus particle and a water molecule.

FIG. 14 shows an EBC concentrator having a semipermeable membrane for separating excess water from EBC to be tested with concentrated virus particles. The system includes a virus 1402, a selectively permeable membrane 1404, and a water 1406.

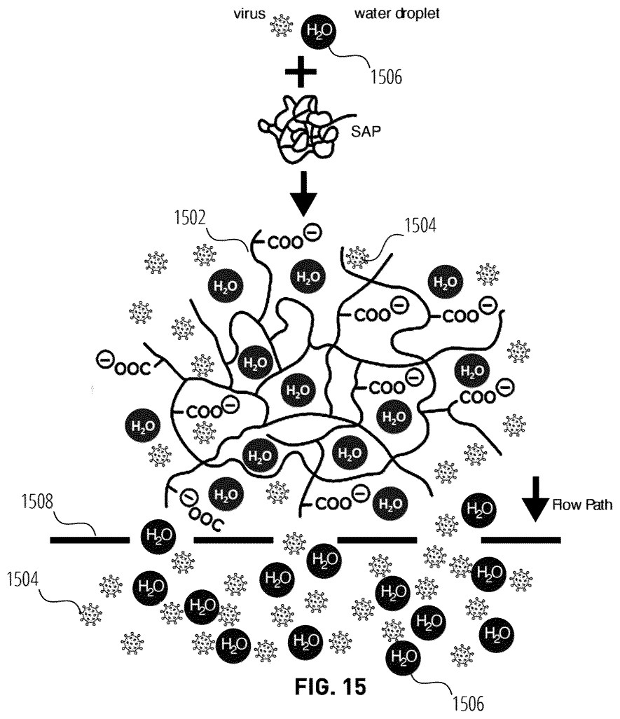

FIG. 15 shows the addition of a super absorbent polymer in the EBC concentrator. The system includes a SAP 1502, a virus 1504, a water 1506, and a selectively permeable membrane 1508.

nanoCLAMPs (nano-CLostridial Antibody Mimetic Proteins) are capture molecules developed by Nectagen (Kansas City, Kansas). Like aptamers and engineered antibodies, these capture molecules can be designed to bind to a specific antigen. nanoCLAMPS are based on an immunoglobulin-like, thermostable carbohydrate binding module from Clostridium hyaluronidase. nanoCLAMPs are small (4 nm×2.5 nm, 15 kDa) and have three variable loops comparable to immunoglobulin complementarity determining regions. nanoCLAMPS are within the Debye screening length and can be designed and enhanced to bind to different locations on the same antigen, different antigens, and large and small target molecules.

Particularly for larger macromolecules, experiments on capture molecules of different sizes indicates that the effectiveness of an FET-based sensor (where antigens are captured via specific binding to capture molecule-functionalized surfaces) is greatly affected by the Debye screening length. nanoCLAMPs typically bind selectively with nanomolar Kd's and release the captured antigen when treated with propylene glycol or glycerol.

For example, a nanoCLAMP can be designed to bind to biomarkers of viruses such as FluA HA, FluB NA, SARS N-protein (more conserved, slower to mutate protein across SARS viruses) and SARS S-protein (faster to mutate, a cause of the SARS-CoV-2 variants). Thus, a multi-biomarker, syndromic testing system can be obtained through a ganged biosensor array that receives the collected EBC biosample. This ganged biosensor can be fabricated as an array of separate biosensor with printed electrodes and/or semi-conductor (e.g., g-FET) designs that are functionalized for a specific use-case through the selection of a respective capture molecule for a corresponding target molecule. In the case of a mask-based diagnostic platform, products made from the platform can share most if not all the components (mask, EBC collector, concentrator, fluid transfer, etc. Since the blood-air exchange that occurs in the lungs produces many biomarkers contained in the EBC, a specific use-case (e.g., tuberculous, lung cancer screening, environmental exposure, health and wellness, fitness, fatigue, vitamin deficiency, etc.) for the mask-based diagnostic system is determined by the addition of biosensors that are functionalized with the appropriate commercially available or custom designed capture molecule.

In accordance with an exemplary non-limiting embodiment, the biomarker concentrator can comprise a super absorbent polymer for preferentially absorbing water from the EBC into polymer chains of the super absorbent polymer. The target biomarker is not absorbed by the polymer chains and flows along with the EBC through the SAP and microfluidics structures of the diagnostic platform. As the EBC flows along through the SAP, the water content in the EBC is removed while the content of the target molecules remains constant, increasing the tested sample concentration of the target molecules.

A testing system includes an exhaled breath condensate (EBC) collector for converting breath vapor received from the lungs and airways of a test subject into a fluid biosample. The elements along the path can be tailored for conditioning the biosample before it is tested. For example, since EBC is mostly water, the target molecule concentration in the tested sample can be improved significantly by removing excess water. A biomarker concentrator concentrates a target biomarker portion in the fluid biosample to form a concentrated fluid biosample. Other test confounding constituents in the EBC can be removed from tested sample. For example, dissolved salts in the EBC can be removed via the SAP and semi/selectively permeable membrane actions described herein, or other constituent removal techniques such as precipitation reactions and filtering can be utilized. A biomarker testing unit receives the concentrated fluid biosample and tests the concentrated fluid biosample for the target biomarker.

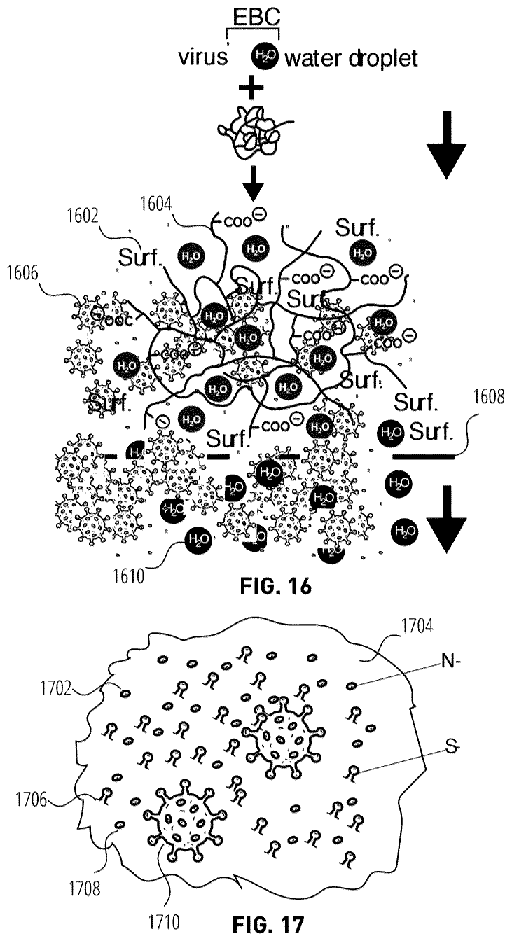

FIG. 16 shows the addition of a surfactant or lysing agent in the EBC concentrator. The system includes a surfactant 1602, a SAP 1604, a virus 1606, a selectively permeable membrane 1608, and a water 1610.

FIG. 17 shows an EBC tested sample having concentrated virus particles and lysed proteins. The system includes a fragment 1702, an exhaled breath vapor 1704, an S-protein 1706, an N-protein 1708, and a virus 1710.

A selectively permeable membrane is provided downstream in the flow path of the EBC sample from the super absorbent polymer. The membrane has a pore size that is configured and dimensioned to allow a portion of water in the EBC sample not absorbed in the super absorbent polymer blend, and the target analyte, to flow through the selectively permeable membrane. The pore size prevents the super absorbent polymer from flowing through the selectively permeable membrane resulting in a concentration of the target analyte in remaining water in the EBC sample on the permeate side of the membrane. The membrane allows virus particles and virus fragments to pass, along with the water not absorbed in the SAP to continue to flow towards the biosensor.

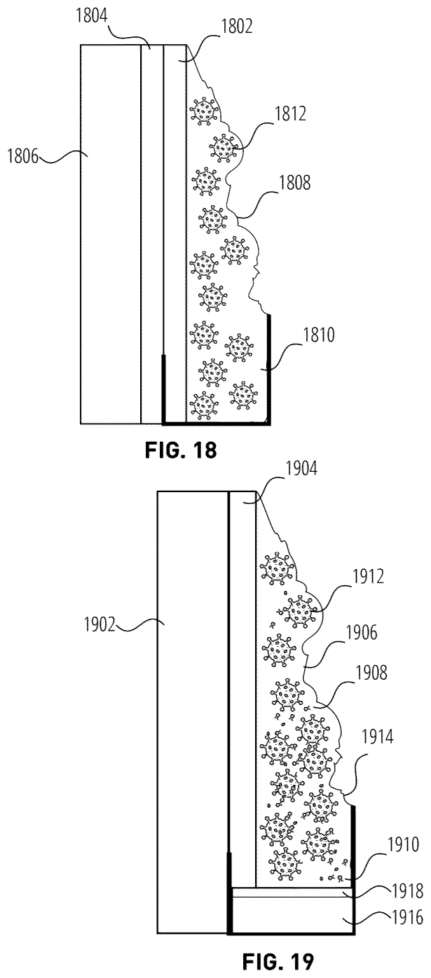

FIG. 18 is a cross section showing an EBC concentrator with a semipermeable membrane and wick disposed adjacent to a thermal mass. The system includes a selectively permeable membrane 1802, a wick 1804, a thermal mass 1806, an exhaled breath condensate 1808, a pooling area 1810, and a virus 1812.

The virus is concentrated in the EBC sample that accumulates at the pooling area formed by a barrier. Excess water in the EBC passes through the semipermeable membrane and is wicked away so that as the exhaled breath vapor is cooled into the EBC that forms on the EBC collector surface, excess water is continuously removed.

FIG. 19 is a cross section showing an EBC concentrator with a fluid conductor with lysing material and having a semipermeable membrane and wick in an EBC pooling area. The system includes a thermal mass 1902, a SAP 1904, an exhaled breath condensate 1906, an N-protein 1908, an S-protein 1910, a virus 1912, a virus-concentrated EBC 1914, a wick 1916, and a selectively permeable membrane 1918.

The biomarker concentrator may comprise a selectively permeable barrier for allowing excess water in the fluid biosample to pass through. The selectively permeable barrier blocks the target biomarker in the fluid biosample from passing through the selectively permeable barrier so that the fluid that is tested has a higher concentration of the target biomarker. An excess water absorbing wick can be provided for absorbing the excess water passing through the selectively permeable material.

Alternatively or in addition, a selectively permeable membrane can be provided upstream in the flow path of the EBC sample from the super absorbent polymer. The membrane has a pore size configured and dimensioned to allow a portion of water in the EBC sample to flow through the selectively permeable membrane to the super absorbent polymer. The pores prevent the target analyte from flowing through the membrane resulting in a concentration of the target analyte in remaining water in the EBC sample on the feed side of the membrane.

In accordance with an exemplary embodiment, an apparatus for testing exhaled breath condensate (EBC) for a target biomarker includes an EBC collector for converting breath vapor received from the lungs and airways of a test subject into an EBC biosample. The EBC biosample contains the target analyte. A biomarker concentrator comprises a super absorbent polymer layer in a flow path of the EBC biosample. During the collection, the EBC biosample sample is contacted with the super absorbent polymer layer and it absorbs a portion of water from the EBC biosample sample. The SAP layer does not absorb the target analyte, resulting in a concentration of the target analyte in remaining water in the EBC biosample. A biomarker testing unit receives the concentrated EBC biosample and tests the concentrated EBC biosample for a target biomarker.

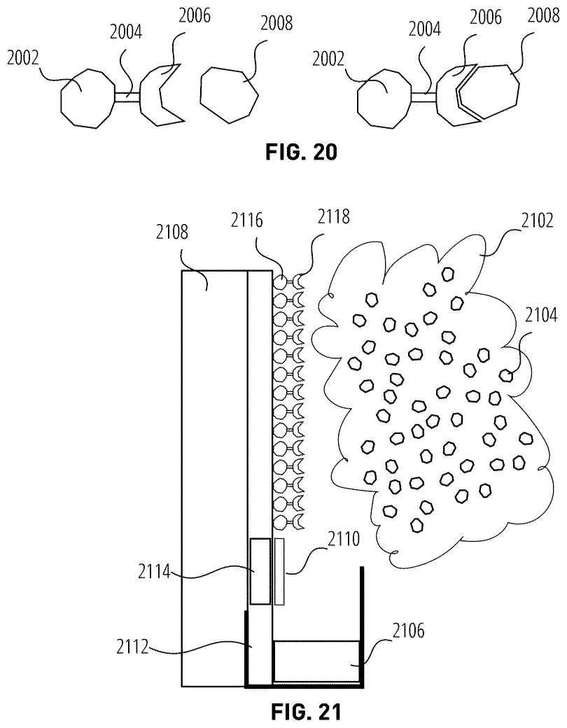

FIG. 20 illustrates a capture molecule/linker/magnetic nanoparticle conjugate (magnetically active molecule) with a free-floating and captured target molecule. The system includes a magnetic nano-particle 2002, a linker 2004, a capture molecule 2006, and a target molecule 2008.

Applied-field-reactive capture molecule conjugates are provided for capturing the target biomarker. The applied-field-reactive capture molecule conjugates have one or more applied-field-responsive ends and one or more capture molecule ends. The capture molecule ends bind to and capture the target biomarker. For syndromic testing, there can be multiple capture molecule types for capturing different target molecules (e.g., S-protein, N-protein of SARS-CoV-2; FluA protein, FluB protein, SARS protein, immuno-responsive marker for inflammation, cytokines, etc.).

The applied-field-reactive capture molecule conjugate may comprise a capture molecule(s) immobilized on a magnetic nanoparticle forming a magnetic nanoparticle conjugate having a magnetically attractive nanoparticle end (mag.NP). As shown in FIG. 21, the dissolvable adhesive can hold the magnetic nanoparticle conjugate in a path of the fluid biosample, the collected EBC. When the water in the fluid biosample dissolves the dissolvable adhesive, the magnetic nanoparticle conjugates become free-floating in the fluid biosample. A wick for absorbing excess EBC constituents and a fluid transfer system (e.g. microfluidic materials, as needed) cause the continuous flow of EBC past the magnetic trap so that the magnetic nanoparticle conjugate and captured target biomarker are concentrated at the magnetic trap and excess EBC constituents that are not held at the magnetic trap flow past the biomarker testing unit to the wick.

FIG. 21 illustrates an EBC collector with oriented and aligned magnetic nanoparticle conjugates on a dissolvable adhesive for capturing and concentrating target molecules in exhaled breath vapor. The system includes an exhaled breath vapor 2102, a target molecule 2104, a wick 2106, a thermal mass 2108, a testing area 2110, a dissolvable adhesive 2112, a magnetic trap 2114, a magnetic nano-particle 2116, and a capture molecule 2118.

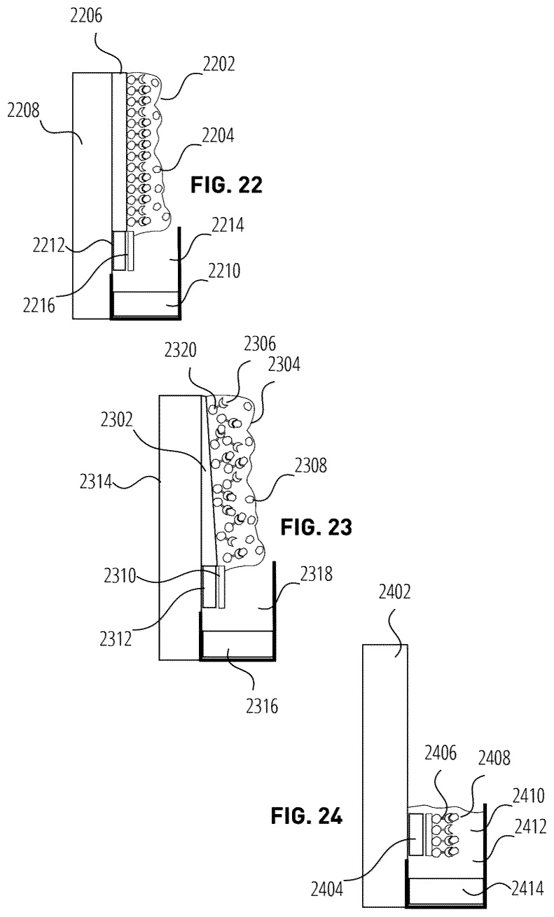

FIG. 22 illustrates the EBC collector with oriented and aligned magnetic nanoparticle conjugates with EBC formed on the surface called by the thermal mass placing the target molecules in the EBC in binding communication with the capture molecules. The system includes an exhaled breath condensate 2202, a target molecule 2204, a dissolvable adhesive 2206, a thermal mass 2208, a wick 2210, a magnetic trap 2212, a pooling area 2214, and a testing area 2216.

FIG. 23 illustrates the water in the EBC dissolving the dissolvable adhesive allowing magnetically active molecule and target molecule conjugates to flow towards a magnetic trap at a testing area. The system includes a dissolvable adhesive 2302, an exhaled breath condensate 2304, a capture molecule 2306, a target molecule 2308, a testing area 2310, a magnetic trap 2312, a thermal mass 2314, a wick 2316, a pooling area 2318, and a magnetic nano-particle 2320.

A dissolvable adhesive holds the applied-field-reactive capture molecule conjugate in a path of the fluid biosample. Water in the fluid biosample dissolves the dissolvable adhesive and allows the applied-field-reactive capture molecule conjugate to be free floating in the fluid biosample. A magnetic trap attracts and holds the magnetically attractive particle end to immobilize the magnetic nanoparticle conjugate and the captured target biomarker at the magnetic trap.

FIG. 24 illustrates the magnetically active molecule and target molecule conjugates held by the magnetic trap at the testing area. The system includes a thermal mass 2402, a magnetic trap 2404, a magnetic nano-particle 2406, a target molecule 2408, an exhaled breath condensate 2410, a pooling area 2412, and a wick 2414.

The magnetic trap is provided in proximity to the biomarker testing area and holds the magnetic nanoparticle conjugates. As the complexes that also include the captured target molecule accumulate, the detectable signal produced in response to the detected captured target molecules increases. Knowing or approximating the flow rate or volume of EBC that arrives at the testing area can be used to quantitatively measure the concentration of the target biomarker. The viral load, or environmental exposure, or progression of disease can be determined as a function of the flow and detectable signal.

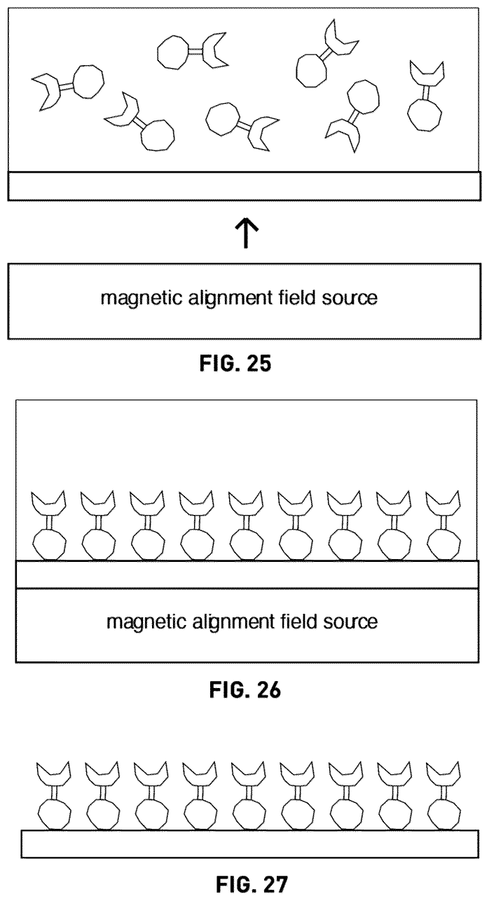

The formation of a well-organized magnetic nanoparticle capture molecule conjugate array is shown fixed on a dissolvable adhesive film. FIG. 25 shows a dissolvable adhesive film supporting a carrier fluid holding free floating and randomly dispersed magnetic nanoparticle conjugates before a magnetic alignment field is applied, where the carrier fluid is a non-solvent for the dissolvable adhesive.

FIG. 26 shows the magnetic nanoparticle conjugates oriented and aligned on the dissolvable adhesive film in the carrier fluid after a magnetic alignment field is applied.

FIG. 27 shows the well-organized magnetic nanoparticle conjugates fixed in place on the dissolvable adhesive film after the carrier fluid is allowed to evaporate and the magnetic alignment field is removed.

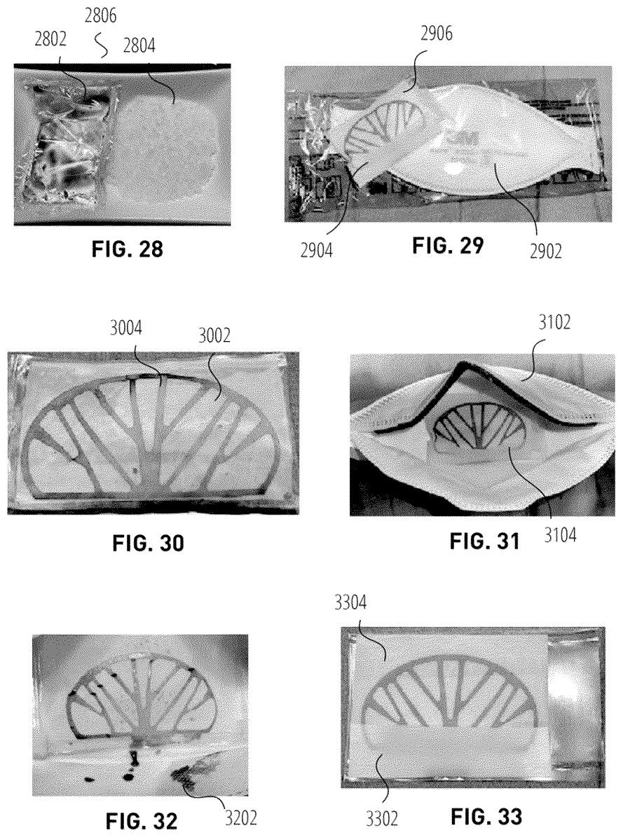

FIG. 28 shows the constituents of an endothermic reaction, including water and urea crystals. Other chemicals that react endothermically with water include ammonium nitrate and calcium ammonium nitrate. The system includes a water bag 2802, urea crystals 2804 which are examples of endothermic materials 2806.

FIG. 29 shows a retrofittable endothermic EBC collector and a pre-existing face mask. The system includes a face mask 2902, an EBC collector 2904, and a testing system support 2906.

FIG. 30 shows an endothermic EBC collector having hydrophilic channels on a hydrophobic field, with a dye incorporated in the hydrophilic channels to simulate a surfactant, precipitation reaction, and/or buffer additive. The system includes a hydrophobic surface 3002 and a hydrophilic structure 3004.

FIG. 31 shows an endothermic EBC collector retrofit into an existing face mask. The system includes a face mask 3102 and a testing system support 3104.

FIG. 32 shows a reacted endothermic EBC collector and dye colored collected EBC droplets formed from the exhaled breath condensate 3202.

In accordance with an exemplary embodiment, the EBC collector includes a condensate-forming surface and a thermal mass in thermal connection with the condensate-forming surface. The thermal mass may comprise at least a first chemical reagent and a second chemical reagent combinable to form an endothermic chemical reaction for absorbing thermal energy from the condensate-forming surface for converting the exhaled breath vapor to the EBC. FIG. 33 shows an endothermic EBC collector with a pooling area and dry buffer/surfactant. The system includes a pooling area 3302 and an EBC collector 3304.

FIG. 34 shows an Exhaled Breath Condensate Collector and a testing area with ganged syndromic testing biosensors. The system includes a fragment 3402 and a testing system support 3404.

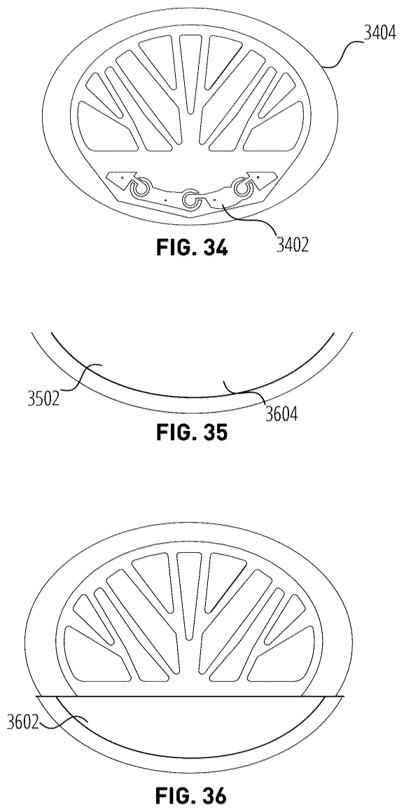

FIG. 35 shows a pooling area for immersing the biosensors in collected EBC, with dry buffer/surfactant in the pooling area. The system includes a pooling area 3502.

FIG. 36 shows an assembled EBC collector and pooling area with dry buffer/surfactant in the pooling area. The system includes a pooling area 3602 and a dry buffer/surfactant 3604.

The fluid transfer system may comprise at least one of a fluid conductor, a pooling area, and a microfluidics transfer path for controlling a flow of the fluid biosample received from the EBC collector. A target biomarker releasing material is disposed in or on said at least one of the fluid conductor, pooling area and microfluidics transfer path. The target biomarker releasing material may include at least one of a surfactant and a chemical lysing agent. The EBC collector can also include a target biomarker releasing structure for mechanically lysing at least one of a cell wall, encapsulating structure and viral envelope containing the target biomarker.

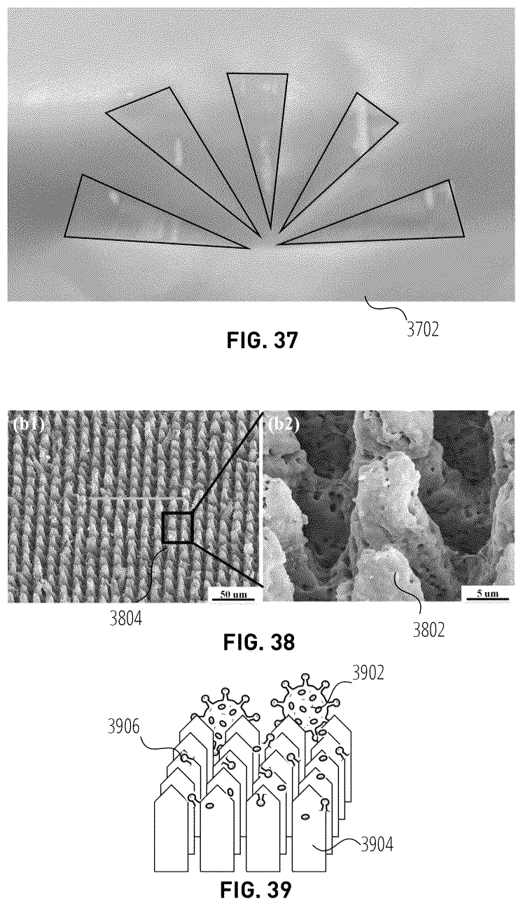

FIG. 37 shows a Teflon condensing surface with a textured fluid conductor. The system includes a patterned teflon sheet 3702.

FIG. 38 is a photomicrograph showing tunable water adhesion structures fabricated by laser ablation taken from a research paper, Superhydrophobic polytetrafluoroethylene surfaces with accurately and continuously tunable water adhesion fabricated by picosecond laser direct ablation, Qin et al., Materials and Design 173 (2019) 107782. The system includes teflon spikes 3802 and a patterned teflon sheet 3804.

FIG. 39 illustrates the tunable water adhesion structures form in a Teflon sheet and having Teflon “spikes” for mechanically disrupting a viral envelope to produce free floating N- and S-proteins of the virus. The system includes a virus 3902, a teflon spikes 3904, and a fragment 3906.

The target biomarker releasing structure may comprise lysing structures protruding from the condensate-forming surface and/or a surface in the flow path of the EBC. The lysing structures mechanically disrupt the cell wall, encapsulating structure and/or viral envelope containing the target biomarker. For example, to release the N-protein from the SARS-CoV-2 virus, the lipid viral envelope can be disrupted using the action of micro- or nano-structures. As the EBC forms and flows along the textured surface, spike-like elements forming the texture interact with the virus and break open the viral envelope. The EBC carrier fluid includes at least one of water, a buffer, a surfactant, a lysing material, a preservative and a body fluid, including at least one of saliva, urine, exhaled breath condensate, blood and sweat. In any case, the biosample is a fluid and the lysing action can be enhanced by providing other micro/nano-grit materials and may require some pressure and friction applied (e.g., by a user's thumb pressing structure on the outside of the mask that grinds the virus particles against the texture structures and/or the the micro/nano-grit material).

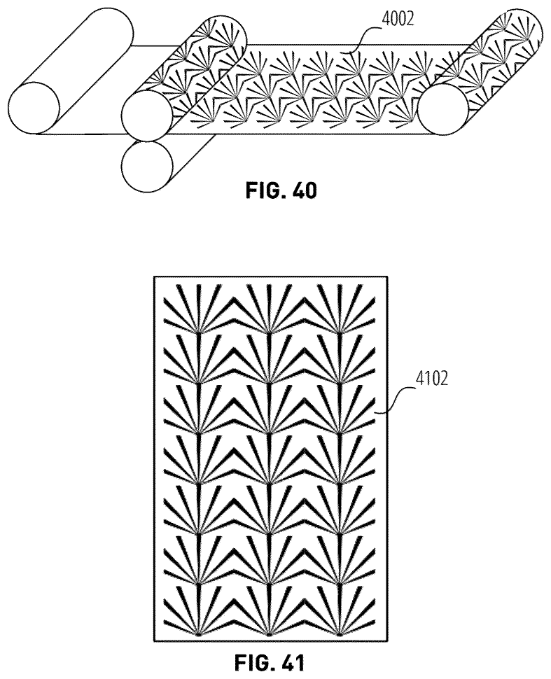

FIG. 40 shows a roll-to-roll process for mass producing a condensing surface with a textured fluid conductor. The system includes a patterned teflon sheet 4002.

FIG. 41 shows a sheet of condensing surfaces with textured fluid conductors. The system comprises a patterned teflon sheet 4102.

The sheet can be mass produced and be part of a materials stack for forming the EBC collector in a high volume manufacturing process, then the individual EBC collectors singulated from a finished roll of completed EBC collectors. The condensate-forming surface may have a relatively low energy surface property for limiting an adhesion of target biomarker to the condensate-forming surface. A fluid conductor disposed on the condensate-forming surface can be provided as a textured structure formed on the condensate-forming surface. The textured structure has a relatively higher energy surface property for guiding a flow of the EBC towards a desired direction.

A method for forming a condensate collector having fluid conductor channels on a substrate for guiding a flow of fluid towards a desired direction comprises providing the substrate having a surface having a relatively lower energy surface property. A textured structure forms fluid conductor channels on the surface having a relatively higher energy surface property for guiding a flow of fluid towards a desired direction.

The relatively lower energy surface property limits an adhesion of a target analyte on the surface and makes the surface relatively hydrophobic, and the higher energy surface property of the textured structure makes the channels relatively hydrophilic. The textured structure can be formed by at least one of laser ablation, sandblasting, etching and calendaring.

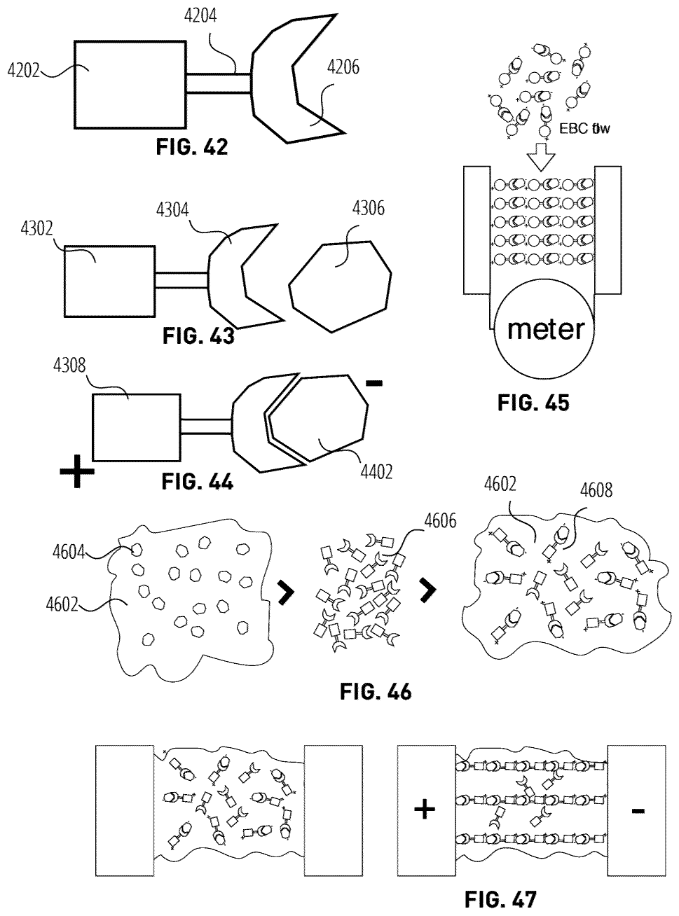

FIG. 42 shows a capture molecule structure having a label attached to a ligand. The system includes a label 4202, a linker 4204, and a capture molecule 4206.

FIG. 43 shows a capture molecule structure having a polarizable end and a ligand end for capturing a target analyte. The system includes a polarizable end 4302, a capture molecule 4304, a target molecule 4306, and a polarizable end 4308.

FIG. 44 shows the capture molecule structure with a captured target analyte forming a polar conjugate. The system includes a target molecule 4402.

FIG. 45 shows the alignment and testing of the polar conjugate to detect the target analyte in a fluid sample.

FIG. 46 shows a carrier fluid comprising a fluid sample with free floating target analyte molecules that is mixed with capture molecule structures to form free floating polar conjugates in the fluid sample. The system includes a carrier fluid 4602, a target molecule 4604, a capture molecule 4606, and a free-floating molecule 4608.

FIG. 47 shows the free-floating polar conjugates before aligning in an applied electric field and the free-floating polar conjugates aligned in the applied electric field.

A method for detecting a target analyte comprises providing a capture molecule structure having a ligand end and a polarizable end. When the capture molecule structure is disposed in a carrier fluid, the capture molecule structure is a free-floating element. A target analyte is provided as another free-floating element in the carrier fluid. The ligand end of the capture molecule structure binds to the target analyte and forms a free-floating polar conjugate having a positive end and a negative end. The polar conjugate is aligned in the carrier fluid in an electric field and an electrical property of the aligned polar conjugate is measured to detect the target analyte.

The carrier fluid can be a body fluid, including at least one of saliva, urine, exhaled breath condensate, blood and sweat. The step of measuring can involve pulsing the electric field for a duration and taking a measurement of the electrical property within a period of time after the duration, where the period of time is short enough to allow detecting the target analyte.

The carrier fluid can be a bio fluid sample and the capture molecule structure provided as a dry powder prior a step of mixing the capture molecule structure with the carrier fluid. The target analyte can be a constituent of the bio fluid sample. Alternatively, the carrier fluid can be an environmental fluid sample, and the target analyte is a constituent of the environmental fluid sample.

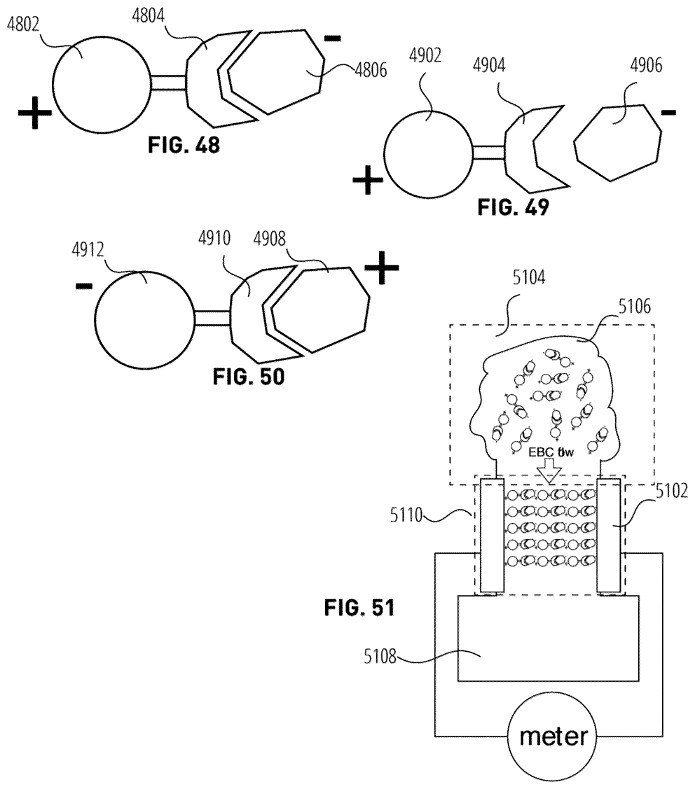

FIG. 48 shows a polarized gold nanoparticle/linker/capture molecule/target molecule conjugate. The system includes a gold nano-particle 4802, a capture molecule 4804, and a target molecule 4806. There is an affinity between the capture and the target molecules, where polarized Au nanoparticles provide a stronger electrochemical affinity for facilitating binding of a capture molecule to a corresponding target molecule.

FIG. 49 shows a polarized gold nanoparticle/linker/capture molecule conjugate where the linker is effective to functionalize the gold nanoparticle to form a positive gold nanoparticle end. The system includes a gold nano-particle 4902, a capture molecule 4904, a target molecule 4906, a target molecule 4908, a capture molecule 4910, and a gold nano-particle 4912.

FIG. 50 shows a polarized gold nanoparticle/linker/capture molecule conjugate where the linker is effective to functionalize the gold nanoparticle to form a negative gold nanoparticle end.

FIG. 51 schematically shows a testing system where EBC containing a target molecule is mixed with a polarizable capture molecule conjugate to form a polarized conjugate, and the polarized conjugate is aligned in a gap between electrodes and the electrical characteristics of the aligned polarized conjugate is used to test for the presence of the target molecule in the EBC. The system includes an electrode 5102, an EBC collector 5104, an exhaled breath condensate 5106, a wick 5108, and a fluid transfer 5110.

The captured target molecule complex can be driven into alignment and orientation in the EBC carrier fluid at the testing area. The organized array of captured target molecule complexes improves the detectable signal and the obtained test results. For example, the applied-field-reactive capture molecule conjugate may comprise a linker molecule disposed between the applied-field-responsive end and the capture molecule end. The linker molecule provides the applied-field-reactive capture molecule conjugate with electro-chemical properties wherein when the capture molecule end binds with the target biomarker at least one of the degree of polarity and the conductivity of the applied-field-reactive capture molecule conjugate changes.

In accordance with an exemplary embodiment, a capture molecule conjugate for detecting a target analyte comprises an applied-field-reactive capture molecule conjugate, where the capture molecule is part of a larger molecule conjugate that is reactive to an applied field. For example, a polar molecule conjugate that has a positive charge end and a negative charge end will orient along electric field lines between two parallel conductors that form an electronic circuit with one of the conductors being an anode and the other conductor being a cathode. The applied-field-reactive capture molecule conjugate has at least one applied-field-responsive end and at least one capture molecule end. The capture molecule end binds to and captures the target analyte. The binding of each capture molecule end to the target analyte increases an electrical charge difference between the applied-field-responsive end and the capture molecule end.

A linker molecule disposed between the applied-field-responsive end and the capture molecule end provides the applied-field-reactive capture molecule conjugate with electro-chemical properties. When the capture molecule end binds with the target analyte, at least one of the degree of the polarity and the conductivity of the applied-field-reactive capture molecule conjugate changes. For example, in the case of an applied-field-reactive capture molecule conjugate with a positively-charged Au nanoparticle end (for example, a AuNP conjugated with a nanoCLAMP capture molecule), the capturing of a negatively charged target molecule (S-protein of SARS-CoV-2) will create a polar analyte-labeled capture molecule complex that has a change in the degree of polarity at the positive end at the AuNP) and a negative end (at the captured S-protein).

The applied-field-responsive end may comprise at least one of a nanoparticle, a gold nanoparticle, a magnetically active nanoparticle, a carbon tube, graphene. The at least one capture molecule end comprises at least one of an engineered antibody, an antibody, an aptamer, a nano body, and a nanoCLAMP, a small molecule, and the like.

### Proof-of-Concept Experimental Results:

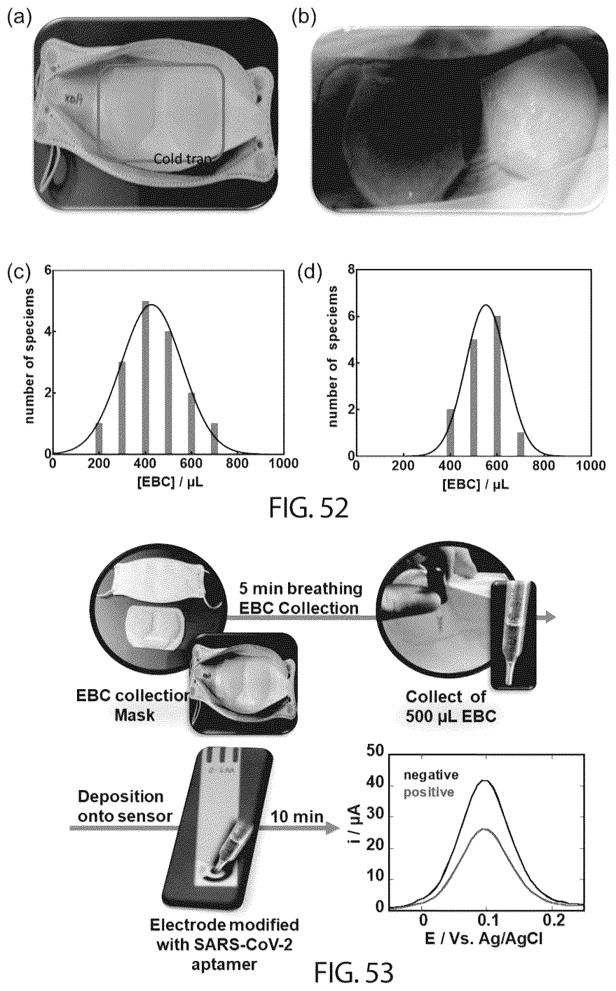

FIG. 52 shows (a) the inside of a proof-of-concept laboratory engineered mask showing an exhaled breath condensate (EBC) collector (cold trap) for converting breath vapor into a fluid sampling. The EBC collector is made of a Teflon-based condensate-forming surface, (b) an image of EBC formed on the Teflon collector after 5 min breathing into the mask, (c) a graph showing EBC volume collected in 5 minutes using the EBC Mask (n=14), and (d) a graph showing EBC volume collected in 5 minutes using a commercial RTube condensers (n=14).

The inventive Exhaled breath condensate (EBC) based diagnostic platform can be used for testing for SARS-CoV-2 infectivity, and for other pathogens and environmental exposures. In proof-of-concept testing, a laboratory engineered mask allows collection of EBC by first cooling the mask for 30 min in the freezer, putting on the cooled mask and breathing into it for 5 min. EBC formed in the Teflon-lining of the inside of the masks is collected and directly deposited onto an electrochemical sensor modified with a SARS-CoV-2 specific aptamer targeting the receptor-binding domain (RBD) region of the S1 spike protein as surface receptor. Using ferrocenemethanol as redox meditator before and after viral interaction allows discrimination between positive and negative EBC samples.

The mask-based EBC collection system is based on a commercial face mask fitted with an engineered EBC collector system based on a Teflon film cooling trap (a). To increase the EBC collection efficiency, the mask is placed into a freezer −20° C. for 30 min, before being placed over the mouth of the person to be tested. This polytetrafluoroethylene (PTFE) trap when cooled allows sample liquification on its surface, where the formed droplets can be collected with a pipette and used for analysis directly. The presence of a collection pool allows further collection of EBC (b) without the need for technical expertise. During the EBC collection the inside of the mask is not exposed to air and the risk of contamination of the EBC samples is negligible. Using this collection system, 400±150 μL of EBC can be collected within 5 minutes (c).

The collection efficiency was comparable to EBC collected by a commercially available RTube condenser (Respiratory Research Inc., USA) (d). The collection efficiency is person-dependent as seen in (c). However, in most cases the required 300 μL needed for further analysis was obtained in this manner. While indeed, collection of an equal volume of saliva is more efficient at a 5 min time span, saliva is a complex sample matrix containing proteases and other variable components that can impact most assays. This includes the potential degradation of the SARS-CoV-2 S1 protein targeted by the aptamer employed in this test. The much cleaner EBC sample is therefore believed to be more suitable and reliable for rapid testing.

FIG. 53 shows an EBC-based diagnostic strategy for testing for SARS-CoV-2 infectivity.

Experimental tests were performed for demonstrating the potential of the inventive diagnostic platform for diagnosing SARS-CoV-2 infection. In the experimental tests, the inventive mask-based diagnostic platform was used to test exhaled breath condensates (EBC) collected by a mask-based sampling device, and detection was performed with a proprietary electrochemical biosensor with a modular architecture that enables fast and specific detection and quantification of COVID-19. The face mask forms an exhaled breath vapor containment volume to hold the exhaled breath vapor in proximity to the EBC collector to enable a condensate-forming surface, cooled by a thermal mass, to coalesce the exhaled breath into a 200-500 μL fluid sample in about 2 minutes. EBC RT-PCR for SARS-CoV-2 genes (E, ORF1ab) on samples collected from 7 SARS-CoV-2 positive and 7 SARS-CoV-2 negative patients were performed. The presence of SARS-CoV-2 could be detected in 5 out of 7 SARS-CoV-2 positive patients. Furthermore, the EBC samples were screened on an electrochemical aptamer biosensor, which detects SARS-CoV-2 viral particles down to 10 pfu mL−1 in cultured SARS-CoV-2 suspensions. Using a “turn off” assay via ferrocenemethanol redox mediator, results about the infectivity state of the patience are obtained in 10 min.

While other human coronaviruses, e.g. HCoV-229E and HCoV-OC43, have only induced mild common cold effects, the SARS-CoV-2 pandemic has caused more than 4.47 M death worldwide (as for 26 Aug. 2021) with 214 M cases detected. Infection with SARS-CoV-2 is diagnosed worldwide using nasopharyngeal swab samples and more recently saliva samples by detection of SARS-CoV-2 RNA using real-time reverse-transcription polymerase chain reaction (RT-PCR). The procedure to obtain nasal swab samples is not only uncomfortable, but requires specialized personal with risk of contaminating the person performing the test. Saliva tests have the advantage of being simpler to perform, less invasive with limited risks and RT-PCR on saliva specimens is becoming more widely implemented. The viscose nature of saliva together with the presence of saliva proteases, responsible for the proteolytic activity of saliva, make the direct application of saliva samples challenging. It is well known that the major mechanisms of COVID-19 spread are airborne and contact infections primarily due to the high resistance of the virus once in aerosol droplets expelled from infected persons. Given the growing need for sample collection by patients themselves, exhaled breath condensate (EBC) might represent an important alternative specimen type for SARS-CoV-2 diagnostic.

The utility of an aptamer-based electrochemical biosensor has been validated by the experimental results presented herein. Aptamers exhibit many advantages as recognition elements when compared to traditional antibodies due their small size, enhanced chemical stability and low cost of production. In the experimental tests, electrochemical sensing was done targeting the spike protein(S) which is embedded in a lipidic membrane forming the SARS-CoV-2 viral outer wall. The spike protein protrudes from the viral membrane, and the viral entry into host cells is mediated by the receptor-binding domain (RBD) region of the spike protein that recognizes the host receptor ACE2. With the spike protein being repeated about 50-200 times on the viral surface, the RBD region of the S protein represents therefore an excellent diagnostic target. The aptamer chosen in this experimental testing is a 32-nucleotide aptamer from Base Pair Biotechnologies (Pearland, Texas, USA).

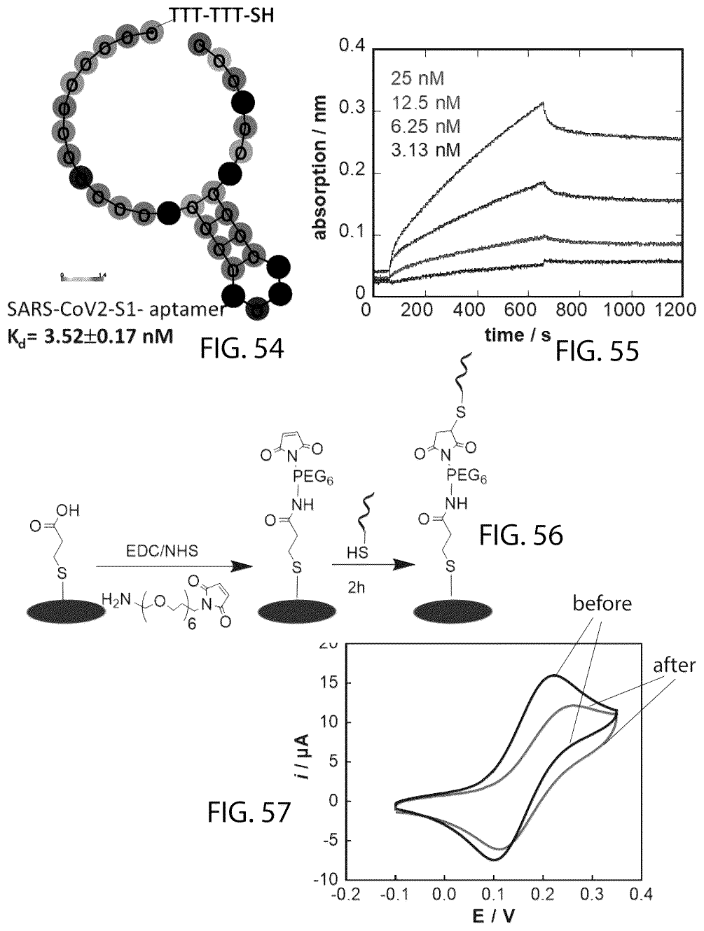

FIG. 54 illustrates a 2D aptamer capture molecule structure (sequence redacted) that was experimentally shown to be effective in the EBC-based diagnostic strategy for testing for SARS-CoV-2 infectivity.

FIG. 55 illustrates biolayer interferometry (BLI) measurements of biotinylated aptamer linked onto streptavidin-activated BLI sensors with different concentrations of SARS-CoV-2 S protein (3.13 nM, 6.25 nM, 12.5 nM and 25 nM): running buffer: 1×PBS, 1 mM MgCl2).

FIG. 56 illustrates a surface attachment strategy of SARS-Cov-2 aptamer on a gold working electrode of a screen-printed electrode using maleimide-thiol-aptamer linkage.

FIG. 57 illustrates cyclic voltammograms (CV) of a gold electrode before and after functionalization with SARS-CoV-2 aptamer (10 μg mL−1 for 2 h) using ferrocenemethanol as a redox mediator (1 mM in 0.1 M PBS, pH 7.4, Scan rate=100 mV s−1).

The SARS-CoV-2 aptamer targeting the S1 protein was selected via combinatorial libraries of nucleic acid sequences by the SELEX (systemic evolution of ligands by exponential enrichment) process. The aptamer investigated is a 20-base aptamer “CFA0688T” (Base Pair Bio) with 1 loop modified on 5′ end with a thiol-TTT-TTT to give the aptamer some flexibility for its anchoring onto gold interfaces. The binding affinity to the recombinant SARS-CoV-2 S1 spike protein was determined by Biolayer interferometry (BLI) measurements and was determined as KD=3.52±0.17 nM (R2=0.9985). This affinity value is comparable to other reported SARS-CoV-2 aptamers such as the 51-base pair aptamer with 3 hair-pined structures selective to RBD or the 58-base pair aptamer with KD values ranging from 5.8±0.8 nM to 0.49±0.05 nM.

The attachment of the SARS-CoV-2 aptamer to screen printed electrodes (SPE) was achieved via a maleimide functionalized poly(ethylene glycol) (PEG) spacer, a commonly employed hydrophilic polymer to avoid biofouling and used for cysteine-modified aptamer integration by others. The spacer aids in overcoming any potential steric hindrance in viral detection. The success of the linking strategy was validated using XPS.

A key concept in electrochemical systems is the fact that the kinetics of the heterogeneous electron transfer at modified electrodes is strongly dependent on the surface coverage and on the thickness of the modifying layer. The cyclic voltammograms of the gold working electrode before and after modification with the aptamer were recorded using ferrocenemethanol as a redox mediator. This small mediator can permeate to a small extent into a monolayer modified gold electrode or via diffusion through pinholes with electron transfer occurring at the free sites on the electrode. As expected, a decrease in electron transfer is observed in line with the presence of the aptamer on the electrode surface

The analysis of 50 nM receptor domain binding from solution on the aptamer-modified electrodes shows a clear decrease in current. This decrease in current is linear up to [RBD]=10 nM, reaching complete saturation at 50 nM. The corresponding curve can be fitted with the Langmuir isotherm using the following equation:

$$Θ = j ( c 0 ) / j ( c ∞ ) = KA \times c 0 / ( 1 + KA \times c 0 )$$

With Θ being the surface coverage, j(c0) the current density at a given RBD concentration, j (c∞) the current density at infinite bulk analyte concentration; assuming a 1:1 complex between the antigen (RBD) from solution and the aptamer receptor allows estimating the affinity constant KA. From the expected S-shaped curve, a dissociation constant (i.e. a half saturation-constant) KD=1.6±0.9 nM could be determined indicating high affinity of the aptamer for RBD, and in line with reported nanomolar dissociation constants for aptamer-protein interactions as well as independent affinity measurements made by Base Pair using biolayer interferometry.

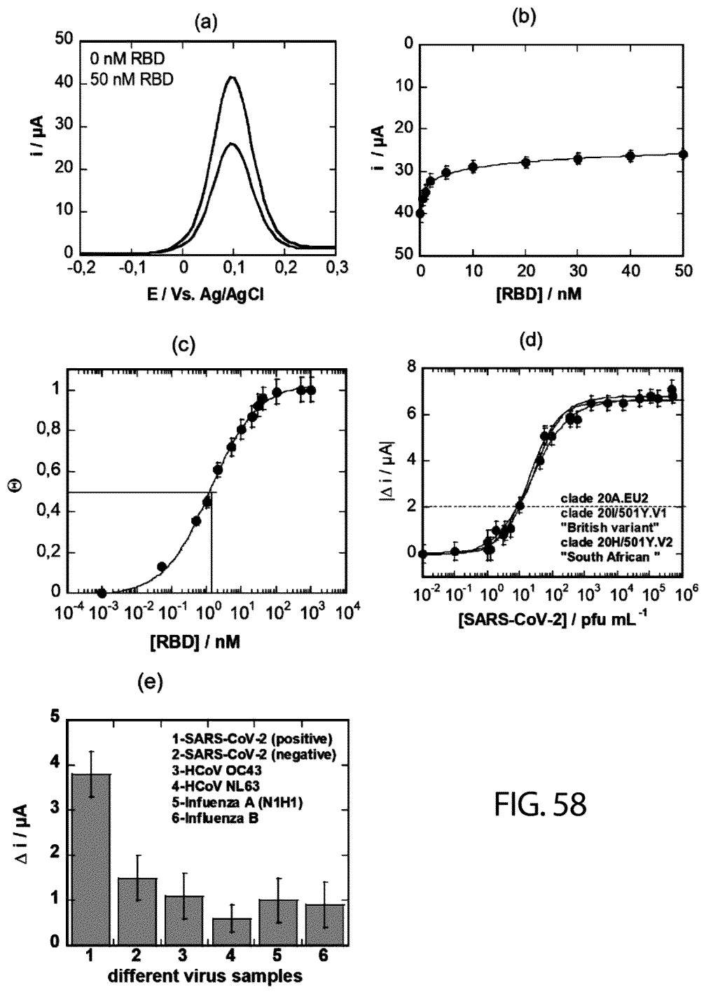

FIG. 58 shows (a) a differential pulse voltammogram of an aptamer electrochemical SARS-CoV-2 aptasensor, (b) current response to increasing binding domain concentrations, (c) Langmuir adsorption isotherm, (d) response curve of different virus variants, and (e) the selectivity of the aptamer sensors.

These interfaces were investigated for their potential to sense cultured SARS-CoV-2 viral particles. Immersion of the sensor into PBS (0.1 M, pH 7=4) containing different concentrations of a SARS-CoV-2 isolate shows that the limit of detection (LOD), defined as the lowest level that an analyte can be reliable distinguished from the background, correlates to about 10 pfu mL−1 (correlating to a current difference of 2 μA) with a saturation at 1.5×105 pfu mL−1. The detection limit was determined to be about 3 pfu mL−1 from five blank noise signals (95% confidential level). The analytical performance was compared to that of SPE where the thiol-terminated aptamer was directly linked onto the gold surface. From the analysis of RBD binding to the aptamer, a higher dissociation constant of KD-6.2±1.2 nM was determined. More importantly sensing of cultured SARS-CoV-2 viral particles indicates a LOD of about 200 pfu mL−1.

This sensing sensitivity of the maleimide-thiol aptamer sensor is comparable to other electrochemical and electrical sensors reported thus far in the literature. In addition, the possibility of detecting the variants 20I/501Y.V1 (called “British variant”) and 20H/501Y.V2 (called “South African variant”) was investigated by using SARS-CoV-2 variant isolates. (c) indicates that the aptamer-based sensor senses the 20I/501Y. V1e variant and the 20H/501Y.V2 variant equally well. This is in line with results using commercial recombinant SARS-CoV-2 S protein considering the different mutations. Indeed using BLI measurements, the affinity of the SARS-CoV-2 S protein UK variant to the aptamer is KD=6.0±3 nM with a kon=(1.52±0.003)×105 M−1s−1 while in the cases of the South African variant is KD=1.6.0±0.1 nM with a kon=(1.62±0.05)×105 M−1s−1. This is in the same order as the affinity constant of 3.52±0.17 nM for the wild type (Wuhan) variant.

The reproducibility of the SARS-CoV-2 aptamer electrodes was expressed in terms of the relative standard deviation, which was determined to be 2.3% at a viral concentration of 103 pfu mL−1 (n=5). The long-term stability of the sensor when stored in PBS was also evaluated showing a loss of 2.5% in the anodic peak current when testing virus solutions of 103 pfu mL−1 solution after the electrode has been stored at 4° C. for 1 month. To illustrate the selectivity of the sensor, the aptamer sensor was incubated with other viral samples, obtained by nasal swabs.

Testing on other coronaviruses producing symptoms close to those associated to SARS-CoV-2, HCoVOC43 and HCoV NL63, showed decreased interaction with the aptamer-interface as identified with a current difference smaller compared to the current difference recorded on a positive nasopharyngeal swab sample. The same was observed for Influenza A (H1N1) and influenza B samples.

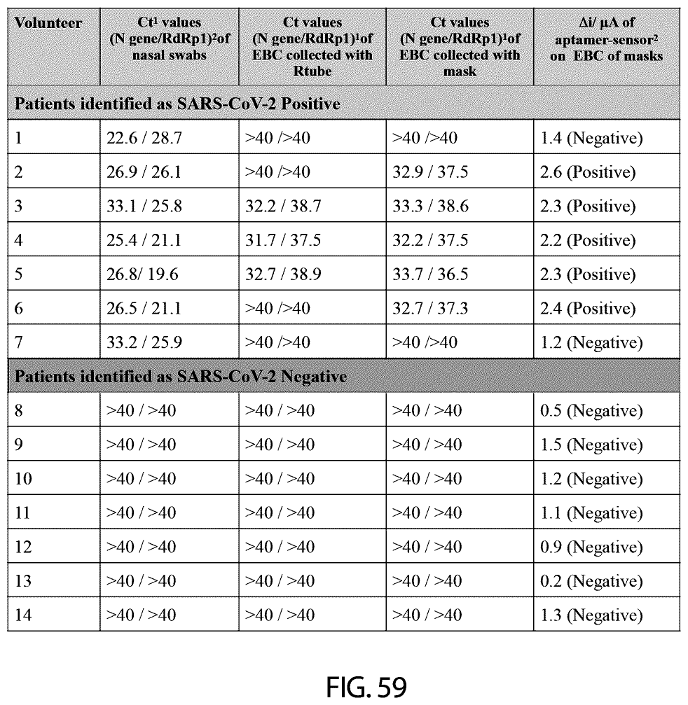

FIG. 59 is a table showing exploratory EBC studies on patients identified by nasopharyngeal swabs RT-PCR as SARS-CoV-2 positive (black numbers) or negative (red numbers).

Given that the disease is transmitted via exhaled droplets, and that EBC is the established modality for sampling exhaled aerosol, detection of SARS-CoV-2 in EBC is a promising approach for safe and efficient diagnosis of the disease. The experimental results validate the possibility of SARS-CoV-2 sensing using EBC collected by commercial Rtube condensers and EBC mask-based system. EBC samples were collected using a cold trap as described herein. In parallel, nasopharyngeal swab samples also were collected.

In a proof of principle study, EBC samples of 14 volunteers were collected and analyzed by PCR as shown in FIG. 60. Out of the 14 nasopharyngeal swab samples, seven were identified as SARS-CoV-2 positive and seven as SARS-CoV-2 negative (Cycle threshold (Ct)>40) by targeting the N structural protein as well as the RNA dependent RNA polymerase (RdRp) nonstructural protein via RT-PCR. Based on the experiment results with different dilutions of a SARS-CoV-2 isolate, it is estimated that a Ct of 34 approximately corresponds to about 104 copies of viral RNA per milliliter and that this dilution showed no infectivity to Vero cells.

The lower Ct values of 22 correlated to about 107 copies of viral RNA per milliliter.

The results of EBC RT-PCR performed on samples collected by Rtube condensers as well as EBC masks of the SARS-CoV-2 negative patients were in full agreement with that of nasopharyngeal swap samples (7/7, 100%). Testing these samples on the electrochemical sensor, where a current difference higher than 2 μA was considered to be linked to the presence of viral particles, resulted in further identification of these samples as SARS-CoV-2 negative.

In the case of EBC samples collected from patients identified by nasopharyngeal RT-PCR as SARS-CoV-2 positive, 3 samples out of 7 were identified as SARS-CoV-2 positive using the commercial RTube condenser and 5/7 using the face mask using a Ct of 40 as cut-off. In contrast to the nasopharyngeal samples, the Ct values of the RdRp gene detected in the EBS samples were always considerably lower than the N-gene. The difference in the Ct values between nasal swab samples and EBC is linked to the different viral load present in both fluids. Indeed, it has been postulated that the viral load of SARS-CoV-2 in aerosol samples is several orders of magnitude below those in nasopharyngeal swabs, which are in the order or 6.41×102-1.34×1011 copies/mL. This indicates that the N-gene is likely the more robust gene to target for EBC samples independently on the collection strategy applied. Testing the mask-collected EBC collected samples on the aptamer electrochemical sensor showed agreement with EBC RT-PCR results using masks. This indicates that such sensors are well-adapted for sensing EBC viral samples and further confirms the presence of active viral particles in exhaled breath of SARS-CoV-2 positive patients.

FIG. 60 illustrates an applied-field-reactive capture molecule conjugate having an applied-field-responsive end and a capture molecule end with a linker molecule providing electro-chemical properties that change at least one of a polarity and a conductivity. The system includes an applied field responsive end 6002, a linker 6004, a capture molecule 6006, and a target molecule 6008.

FIG. 61 illustrates a capture molecule conjugate having a magnetically attractive end and a capture molecule conjugate having a coated AuNP end. The system includes a magnetic nano-particle 6102 and a gold nano-particle 6104.

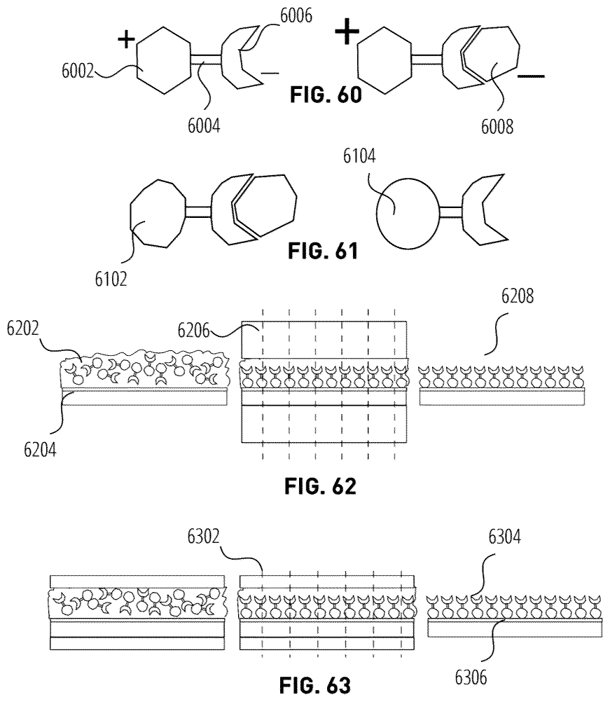

FIG. 62 shows a process for forming aligned and oriented capture molecule conjugates aligned in a magnetic field on a dissolvable adhesive. The system includes a carrier fluid 6202, a dissolvable adhesive 6204, a magnetic field 6206, and an aligned magnetic conjugate 6208.

FIG. 63 shows a process for forming aligned and oriented capture molecule conjugates aligned in an electric field on a dissolvable adhesive. The system includes an electric field 6302, an aligned polarized conjugate 6304, and a dissolvable adhesive 6306.

In accordance with an exemplary embodiment, an array of applied-field-reactive capture molecule conjugates is made by providing a dissolvable adhesive film, on a substrate, liner, or free standing. A carrier fluid that is a non-solvent for the dissolvable adhesive film has randomly dispersed applied-field-reactive capture molecule conjugates. An aligning field is applied to the carrier fluid for assembling the applied-field-reactive capture molecule conjugates onto the dissolvable adhesive film. The carrier fluid is removed (evaporated, dip or spin coating) leaving the assembled applied-field-reactive capture molecule conjugates fixed on the dissolvable adhesive film.

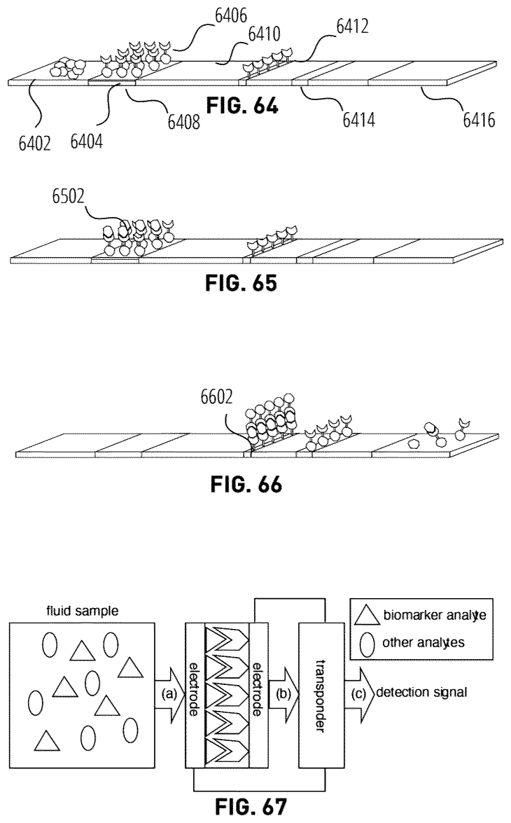

FIGS. 64-66 show a non-limiting exemplary lateral flow assay embodiment where an EBC or other liquid sample containing a target biomarker(s) flows through a multi-zone transfer medium through capillary action. The zones are typically made of polymeric strips enabling molecules attached to the strips to interact with the target biomarker. Usually, overlapping membranes are mounted on a backing card to improve stability and handling. The EBC biosample containing the target biomarker and other constituents is ultimately received at an adsorbent sample pad which promotes wicking of the fluid sample through the multi-zone transfer medium.

As shown in FIG. 64, the system includes a sample pad 6402, a dissolvable adhesive 6404, an aligned magnetic conjugate 6406, a conjugate release pad 6408, a membrane 6410, a test line 6412, a control line 6414, and a wick 6416. The EBC fluid sample including analytes or target biomarkers in a fluid carrier, such as water, is first received at a sample pad which may have buffer salts and surfactants disposed on or impregnated into it to improve the flow of the fluid sample and the interaction of the target biomarker with the various parts of the detection system. This ensures that the analytes will bind to capture reagents as the fluid sample flows through the membranes. The sample pad may be disposed in a pooling area of the EBC collector as shown, for example, in FIG. 12, or a collected EBC sample can be obtained from the pooling area by a pipette and transferred to an LFA.

The treated sample migrates from the sample pad through a conjugate release pad. The conjugate release pad contains labeled antibodies or other capture molecules that are specific for binding with the target biomarker or analytes, and are typically conjugated to colored or fluorescent indicator particles. The indicator particles are typically colloidal gold or latex microspheres. In accordance with an embodiment, an indicator particle is included with a magnetic nanoparticle conjugate, and an array of magnetic nanoparticle conjugates are provided having a magnetically attractive particle end that is fixed to a dissolvable adhesive or otherwise removably immobilized on the conjugate release pad. The magnetically attractive particle may serve as the indicator particle, or colloidal gold or latex microspheres can be additional constituents in the magnetic nanoparticle conjugates.

As shown in FIG. 65, at the conjugate release pad, the labeled capture molecules, indicator particles and target analytes bind to form a analyte-labeled capture molecule complex. The system includes a target molecule-labeled capture molecule conjugate 6502.

The EBC fluid sample dissolves the dissolvable adhesive or otherwise displaces the magnetic nanoparticle conjugates with the captured biomarkers or analytes from the conjugate release pad. Thus, if a biomarker or analyte is present, the fluid sample now contains the analyte conjugated to the labeled capture molecule conjugates. That is, the capture molecule is bound to the target biomarker to form an analyte-labeled capture molecule complex. These complexes along with separate labeled capture molecule conjugated to the indicator particles and magnetically reactive particles that have not been bound to the target biomarker or analyte migrate with the EBC fluid sample along the membrane into a detection zone.

As shown in FIG. 66, the detection zone is typically a nitrocellulose porous membrane and has specific biological components (usually antibodies or antigens) disposed on or impregnated in it forming a test line zone(s) and control line zone. The system includes a test line 6602.

The biological components react with the analyte-labeled capture molecule complex. For example, the analyte-labeled capture molecule complex will bind to a specifically selected primary antibody that is disposed at the test line through competitive binding. This results in magnetic, colored or fluorescent indicator particles accumulating at the test line zone making a detectable test line that indicates the target biomarker is present in the fluid sample.

The primary antibody does not bind to the constituents in the EBC sample that are not bound to an analyte or target biomarker, and these continue to flow along with the fluid sample. At a control line zone, typically a secondary antibody binds with the separate labeled capture molecules conjugated to the indicator particles and thereby indicates the proper liquid flow through the strip. However, in accordance with an embodiment, instead or requiring the additional reagents of the secondary antibodies, a magnetic control line can be provided so that the separate labeled capture molecule conjugated to the indicator particles without the bound target molecules are accumulated by magnetic attraction to the magnetically attractive particles of the magnetic nanoparticle conjugates.

The fluid sample flows through the multi-zone transfer medium of the testing device through the capillary force of the materials making up the zones. To maintain this movement, an absorbent pad is attached as the end zone of the multi-zone transfer medium. The role of the absorbent pad is to wick the excess reagents and prevent back-flow of the fluid sample.

The constituents are selected and disposed on the membranes so that if there is no target biomarker or analyte present in the fluid sample, there will be no analyte-labeled capture molecule complex present that flows through the test line zone. In this case there will be no accumulation of the magnetic, colored or fluorescent particles and no detectable test line will form. Even if there is no biomarker and thus no test line, there will still be a control line formed because the secondary antibody still binds to the separate labeled antibodies that flow along with the fluid sample and/or the magnetically attractive particles of the magnetic nanoparticle conjugates are held at the magnetic control line.

The test and control lines may appear with different intensities depending on the device structure and the indicator particles can be assessed by eye or using an optical or other electronic reader. Multiple biomarkers can be tested simultaneously under the same conditions with additional test line zones of antibodies specific to different biomarkers disposed in the detection zone in an array format. Also, multiple test line zones loaded with the same antibody can be used for quantitative detection of the target biomarker. This is often called a ‘ladder bars’ assay based on the stepwise capture of colorimetric conjugate-antigen complexes by the immobilized antibody on each successive line. The number of lines appearing on the strip is directly proportional to the concentration of the target biomarker.

FIG. 67 shows another testing system that can be used with the inventive EBC collection system that uses an electronic biosensor.

An electronic biosensor has the potential of a much higher sensitivity and can be used to provide a direct-to-electrical signal to enable, for example, easy wireless connectivity. The inventive EBC collection system with an electronic biosensor is easily deployable as a compliment to existing Contact Tracing APPs. The nanoscale dimensions mean many detectors are made at once on a single wafer or through a high volume roll manufacturing process, for lower cost, high throughput manufacturing.

FIG. 67 shows the mechanism of a biosensor detection system. Simplistically, the main components of the biosensor include a sample source (a); a biosensor area that is functionalized with a biomarker-specific bioreceptor (b); and a transducer for generating a readable signal (c). The bioreceptor is matched to a specific target biomarker for lock and key selectivity screening.

In accordance with an embodiment, an applied-field-reactive capture molecule conjugate is provided for capturing the target biomarker. The applied-field-reactive capture molecule conjugate has at least one applied-field-responsive end and a capture molecule end that binds to and captures the target biomarker. The applied-field-reactive capture molecule conjugate can comprise a linker molecule disposed between the applied-field-responsive end and the capture molecule end. The linker molecule provides the applied-field-reactive capture molecule conjugate with electro-chemical properties so that when the capture molecule end binds with the target biomarker at least one of a polarity and a conductivity of the applied-field-reactive capture molecule conjugate changes. For example, the capture molecule end binds to and captures the target analyte. The binding to the target analyte increases an electrical charge difference between the applied-field-responsive end and the capture molecule end.

In accordance with this embodiment, a capture molecule structure thus has a ligand end and a polarizable end. When the capture molecule structure is disposed in a carrier fluid, such as EBC, the capture molecule structure is a free-floating element. A target analyte in the EBC is another free-floating element. The ligand end of the capture molecule structure binds to the target analyte and forms a free-floating polar conjugate having a positive end and a negative end. This free-floating polar conjugate can be aligned the EBC carrier fluid in an electric field and an electrical property of the aligned polar conjugate measured to detect the target analyte.

By this construction of the capture molecule, biosensor and testing system, a fluid sample with some concentration of the target biomarker (possibly as small as a single molecule) flows onto the biosensor field. Some of the biosensor “locks” receive the biomarker “keys.” This causes a detectable change in the output of the transducer that transforms the biosensor output into a readable signal for amplification and data processing.

For example, the desired biomarker can be an antibody that indicates the recovery from a

Covid-19 infection. A fluid sample can be received as a droplet of sweat, nasal swab, blood droplet, or breath condensate, or other body fluid and if the target antibody is present in the sample it interacts with the biomarker-specific bioreceptor. The bioreceptor outputs a signal with defined sensitivity and the transducer generates, for example, a change in an electrical characteristic such as conductivity, indicating the presence of the antibody biomarker in the fluid sample.

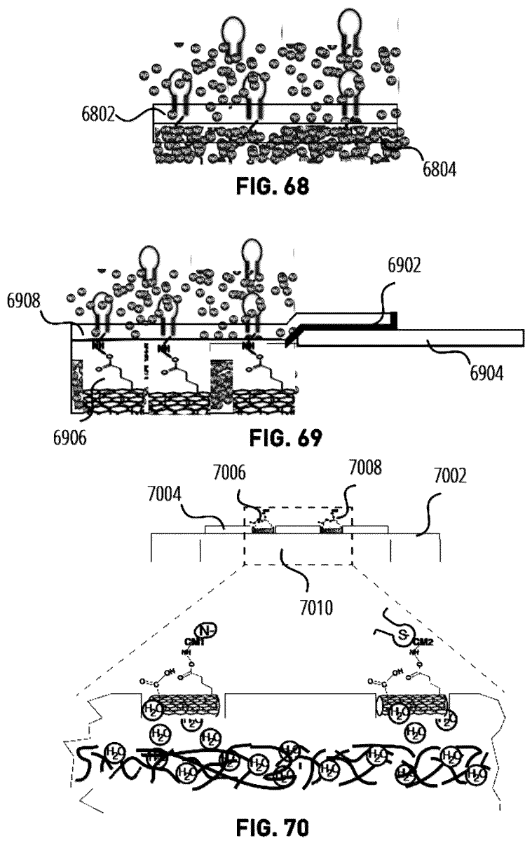

FIG. 68 shows a biosensor constructed on a water absorbing substrate comprising at least one of a selectively permeable membrane and super absorbent polymer fibers, where excess water in a fluid sample is absorbed by the substrate leaving behind a greater concentration of target biomarkers in the tested fluid sample. The system includes a biosensor on water permeable substrate 6802 and a selectively permeable membrane 6804. In accordance with an exemplary embodiment, an electronic biosensor comprises a substrate having a water absorbing property provided by at least one of a selectively permeable membrane, a super absorbent polymer, a microfluidic material, and a wick.

In accordance with an exemplary embodiment, the concentration of a target analyte in a fluid sample, such as an exhaled breath condensate (EBC) sample is increased. An EBC sample containing the target analyte is collected from the lungs and airways of a test subject. A super absorbent polymer is disposed in a flow path of the EBC during the collection. The EBC sample is contacted with the super absorbent polymer which absorbs a portion of water from the EBC sample. The super absorbent polymer does not absorb the target analyte resulting in a concentration of the target analyte in remaining water in the EBC sample.

The super absorbent polymer is provided as a fiber. The fiber can be a melt spun blend of a conventional synthetic material and a super absorbent polymer. The fiber can be pressed into a substrate onto which biosensor electrodes are deposited through vacuum deposition, screen printing, or other process. The electrodes can be functionalized with the capture molecule conjugate using the methods described herein or other sensor fabrication technique.

EP Application: 90311692.9, filed on 24 Oct. 1990, discloses a melt spun fiber blend of a conventional synthetic material and a super absorbent polymer and a process for its production. The blends may be selected from the group consisting of polyethylene, polypropylene, copolymers of a polyethylene, vinyl acetate, and cellulose acetates blended with a super absorbent polymer. These super absorbent fibers are disclosed as being useful in wound dressings, athletic clothing, and other such uses where high water absorbance of a fiber is important.

In accordance with an aspect of the present invention, a super absorbent fiber comprising a blend of synthetic material, such as polyethylene, polypropylene, copolymers of a polyethylene, vinyl acetate, and cellulose acetates, and spun with a super absorbent polymer, such as sodium polyacrylate is provided for concentrating an analyte or target biomarker in a fluid biosample. A wick is formed from the super absorbent fiber that draws excess water out of a fluid biosample, such as exhaled breath condensate.

The research paper Carbon nanotube-immobilized super-absorbent membrane for harvesting water from the atmosphere, Roy et al., Environ. Sci.: Water Res. Technol., 2015, 1, 753 describes a carbon nanotube (CNT)-immobilized membrane for harvesting pure water from air. The CNTs are incorporated into a layer of super-absorbing poly(acrylamide-coacrylic acid) which was cast over a porous hydrophilized polypropylene support. The super-absorbing polymer binds to the water molecules to form water clusters. The paper reports that the incorporation of CNTs led to the interruption of specific water-polymer as well as water-water interactions to generate more free water which permeated more easily through the membrane. The CNTs were functionalized with carboxylic groups to improve the dispersibility into the polymer matrix. The water vapor extraction efficiency reached over 50%, and the presence of CNTs led to an enhancement in water vapor removal by as much as 45% and in the mass transfer coefficient by 44%.

FIG. 69 shows a sample pad of a testing system, such as a lateral flow assay, with a target molecule concentrating structure comprising carbon nanotubes-immobilized super-absorbent and selectively permeable membrane, a SAP/wick and a dissolvable fluid dam. In accordance with a non-limiting embodiment, a sample pad is provided at a first stage of the lateral flow assay. The system includes a dissolvable fluid dam 6902, a conjugate release pad 6904, a wick 6906, and a sample pad 6908.

A selectively permeable membrane is provided adjacent to the sample pad for allowing a portion of a fluid sample to pass through the sample pad and for blocking at least some target molecules to accumulate target molecules in another portion of the fluid sample that does not pass through the selectively permeable membrane. A fluid absorber is provided in fluid communication with the selectively permeable membrane to absorb the portion of the fluid sample that passes through the selectively permeable membrane. A dissolvable fluid dam holds the portion of the fluid sample having accumulated target molecules from flowing to a next stage of the lateral flow assay. Once the dissolvable fluid dam dissolves, a fluid sample with accumulated target biomarkers flow through the constituent parts of the lateral flow assay and test the fluid sample for the target biomarker.

FIG. 70 shows a flow-through sensor electrode construction having one or more capture molecules functionalized on an electron transfer constituent at a detection interface, such as carbon nanotubes, where the electrodes are formed on a water absorbing substrate comprised of a selectively permeable membrane and SAP/capillary fiber composite wick. The system includes a selectively permeable membrane 7002, an electrode 7004, a capture molecule1 7006, a capture molecule2 7008, and a SAP 7010.

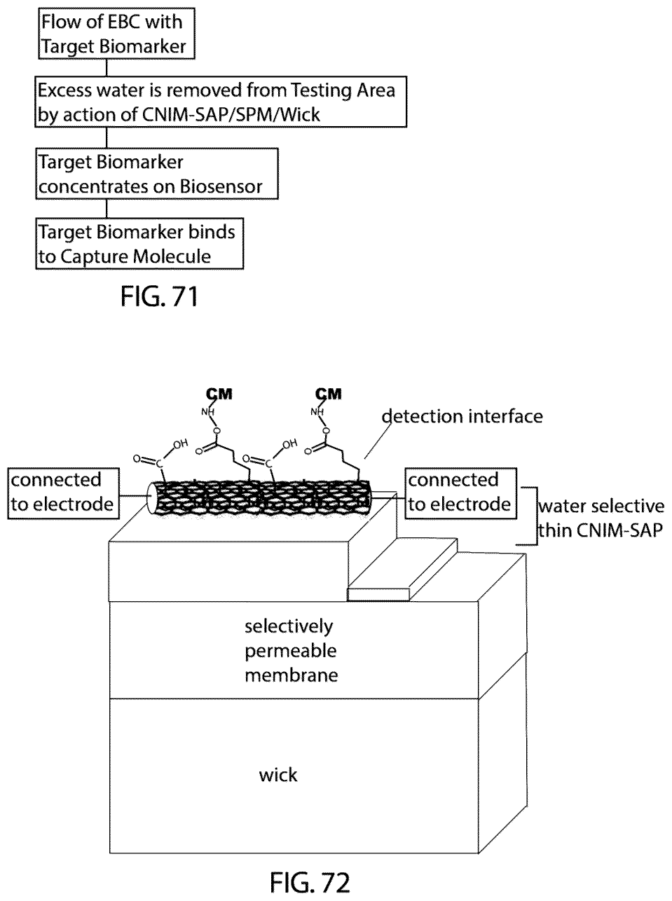

FIG. 71 is a flow chart showing the steps of concentrating a target biomarker in an EBC fluid biosample using an electronic biosensor comprising a substrate having a water absorbing property provided by at least one of a selectively permeable membrane, a super absorbent polymer, a microfluidic material, and a wick. A flow of EBC with a target biomarker is received (step one), excess water is removed by the action of selectively absorbing structure (step two) to concentrate the target molecule in the tested EBC sample received at the detection interface of a biosensor (step three), where the target biomarker binds to a capture molecule immobilized at the detection interface (step four).

FIG. 72 shows an electronic biosensor having a carbon nano-tube immobilized super-absorbent structure for concentrating a target biomarker in an EBC fluid biosample.

In accordance with a non-limiting exemplary embodiment, an electronic biosensor comprises a substrate having a water absorbing property provided by at least one of a selectively permeable membrane, a super absorbent polymer, a microfluidic material, and a wick. At least two electrodes are formed on a top surface of the substrate defining a gap there between. A functionalized detector provided in the gap comprises an electron transport material and a capture molecule. A target molecule captured by the capture molecule causes a change in at least one of a polarity and conductivity of the electron transport material. The target molecule is detected by testing for the change in the polarity or conductivity of the electron transport material. The target molecule that is detected depends on the capture molecule, so this electronic biosensor construction can be utilized to test for many different diseases and other use-cases by changing the capture molecule to match a known biomarker for a corresponding disease or use-case.

The target molecule may be an element of a capture molecule conjugate comprising at least a linker and the capture molecule. The linker bonds the capture molecule to the electron transport material. The electron transport material may comprise carboxylated carbon nanotubes incorporated in a layer of super-absorbing polymer, where the electron transport layer is cast over a porous hydrophilized polypropylene support and provided on the substrate. The functionalized target biomarker detected can be patterned on the hydrophilized polypropylene support and the electrodes formed on a top surface of the hydrophilized polypropylene support. The biomarker concentrator may include a lysing material incorporated with the super absorbent polymer for releasing the target biomarker from a biological element comprising at least one of a virus, a cell, and a bacteria. The target molecule may be an element of a capture molecule conjugate comprising at least a linker and the capture molecule. The linker bonds the capture molecule to the electron transport material. The electron transport material may comprise carboxylated carbon nanotubes incorporated in a layer of super-absorbing polymer, where the electron transport layer is cast over a porous hydrophilized polypropylene support and provided on the substrate. The functionalized target biomarker detected can be patterned on the hydrophilized polypropylene support and the electrodes formed on a top surface of the hydrophilized polypropylene support.

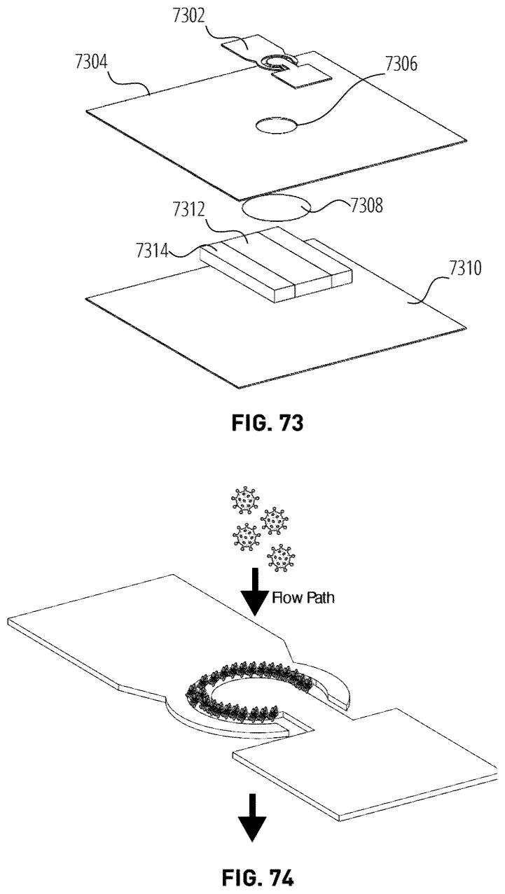

FIG. 73 shows an exploded view of a target molecule concentring flow-through electrode structure. The system includes an electrode 7302, a superstrate 7304, a drainage hole 7306, a selectively permeable membrane 7308, a substrate 7310, a cellulose sponge 7312, and a SAP 7314.

FIG. 74 shows the flow path of a fluid sample with the target biomarker blocked at the sensor electrodes and excess water flowing past the electrodes.

In accordance with a non-limiting embodiment, a sensor comprises a waterproof superstrate with at least a first and second electrode defining a gap there between. The sensor detection interface comprises capture molecules provided in the gap. A drainage hole is formed in the superstrate near the gap and a selectively permeable membrane or super absorbent polymer spun fiber, etc., is provided at the drainage hole for allowing a portion of a fluid sample to pass and for blocking at least some target molecules. A fluid absorber is provided in fluid communication with the selectively permeable membrane to absorb the portion of the fluid sample.

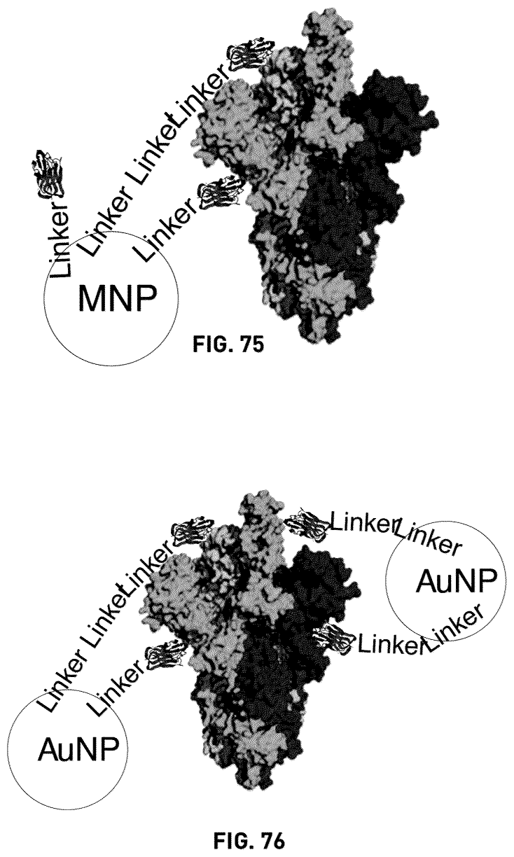

FIG. 75 shows nanoCLAMP capture molecules immobilized on a magnetically active nanoparticle with two capture molecules (e.g. nanoCLAMPs) binding with different binding sites of a protein molecule.

FIG. 76 shows two gold nanoparticles with immobilized nanoCLAMP capture molecules binding with the same target protein molecule.

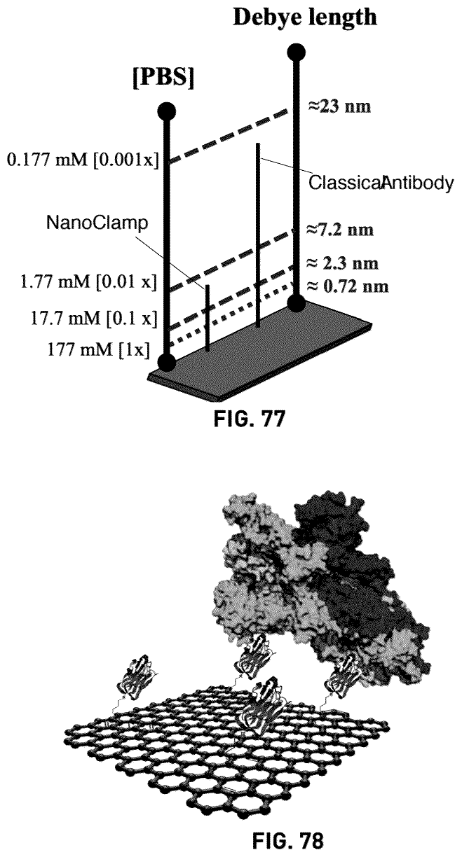

FIG. 77 shows the relative sizes of a nanoCLAMP and classical antibody capture molecule compared to the Debye length.

FIG. 78 shows a monolayer of graphene of an electrolyte-gated graphene field-effect transistor. The graphene layer is functionalized with nanoCLAMP capture molecules through a pyrene linker, where two or more nanoCLAMP capture molecules are binding to different binding sites of a target protein molecule.

As an example, the testing unit provided in a mask-based diagnostic system may comprises a g-FET biosensor having a detection interface comprising a graphene layer functionalized with capture molecules, wherein the capture molecules are smaller than the Debye screening length. A mask-based syndromic testing device including biosensors can be designed to bind to biomarkers of FluA, FluB, SARS N-protein (more conserved, slower to mutate protein across SARS viruses) and S-protein (faster to mutate, cause of the SARS-CoV-2 variants).

In a non-limiting exemplary embody, an electrolyte-gated graphene field-effect transistor is used as the biosensor, with nanoCLAMP capture molecules immobilized on a monolayer graphene structure provided at a detection interface of the sensor. In an electrolyte-gated graphene field-effect transistor, an applied gate voltage leads to an interfacial charge separation between the electrolyte and the graphene channel, with a diffusive double-layer, serving as a dielectric layer on top of graphene. The distance between the two charged layers is usually called Debye length. This length is highly influenced by the ionic strength of the medium. If this is too high, the counter ions present in solution may shield the molecular charges that are away from the gate insulator. As such, this length should be preferably wide enough to detect the interaction between a sensing probe and a charged analyte. The dependency of the Debye length at a solution/gate interface based on the electrical double layer (EDL) with the ionic strength of the electrolyte solution is expressed by:

$${λ}_{D} = \sqrt{\frac{{ε}_{0} {ε}_{r} {k}_{B} T}{2 {NAe}^{2} I}} \qquad ( 1 )$$

where I is the ionic strength of the electrolyte, ε0 is the permittivity of free space, εr is the dielectric constant of the electrolyte, kB is the Boltzmann constant, T is the absolute temperature, NA is the Avogadro number, and e is the elementary charge. The ionic strength of an electrolyte I is expressed by the equation (2):

$$I = \frac{1}{2} \sum {c}_{i} {z}_{i}^{2} \qquad ( 2 )$$

where Σ is the sum of all ions in the electrolyte; i is the total number of ion species; zi is the number of the charges carried by the ion and ci is the ion concentration, which determines the ionic strength of an electrolyte.

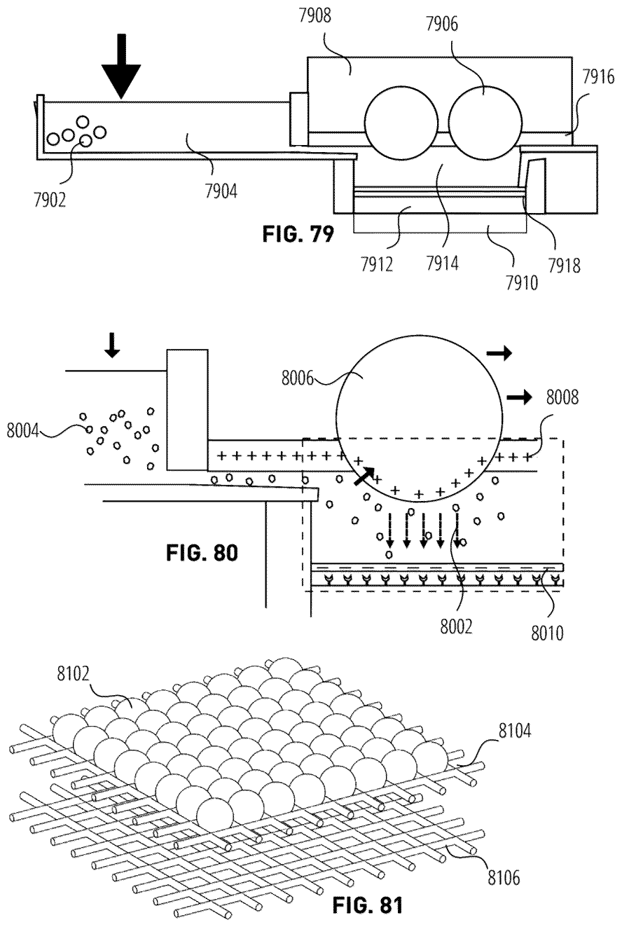

In accordance with a non-limiting embodiment, shown for example, in FIG. 79, temperature T can be increased by a heating element provided adjacent to the detection area of the sensor. This heating element can be a resistive device, where the local temperature of the detection area is increased to speed and improve the binding of capture molecules to target molecules.

According to equations (1) and (2), the Debye length (λD) of a 0.001×PBS solution (I=0.177 mM) is estimated at about 23 nm, which is larger than those of 0.01×PBS (I=1.77 mM, λD=7.2 nm), 0.1×PBS (I=17.7 mM, λD=2.3 nm), and 1×PBS (I=177 mM, λD=0.72 nm). The lower the electrolyte concentration the smaller the ionic strength and the larger λD is.

EBC contains less ions than serum, saliva and sweat and can be considered to have an ionic strength equal to 0.01×PBS (I=1.77 mM, λD=7.2 nm) which makes EBC a superior biosample matrix for GFET sensing, especially if the receptor/analyte interaction is less than about 7.2 nm. With a nanoCLAMP capture molecule of about 4 nm size this superior receptor/analyte interaction is readily achieved (as compared to a classical nanobody/antibody capture molecule). Classical nanobodies having about 13 nm in length results in an inferior GFET sensing as compared to the use of a smaller sized nanoCLAMP as the capture molecule.

### Experimental Results:

A graphene field-effect transistor (g-FET) was constructed, for example, to be functionalized with a nanoCLAMP capture molecule designed for capturing the S-protein of the SARS-CoV-2 virus. Graphene is first synthesized in-house as described below. Interdigitated microelectrodes (ED-IDE1-Au w/o SU8) are provided by Micrux Technologies.

Graphene synthesis: The monolayer graphene is grown by chemical vapor deposition (CVD) on commercial Cu foil from Alpha Acsar (high purity—99.9999%). Graphene growth is carried out in a Jipelec JetFirst Rapid Thermal CVD (RTCVD). This system allows heating and cooling at high rates (10° C. s−1). The growth itself comprises heating, annealing, growth and cooling steps. We used a mixture of 100 sccm of argon and 5 sccm of dihydrogen during all the steps and 20 sccm of methane as a precursor during the growth phase. We first cut the Cu foil in small pieces (2.5×2.5 cm), clean them with acetic acid, acetone and IPA for 5 min each under ultrasonication in order to remove all possible copper oxide and to produce the cleanest surface possible. We then put the pieces onto a Si wafer in the chamber. We proceed to a high vacuum (<5×10−5 bar) before starting and then the sample is heated for 5 min from room temperature to 300° C., followed by 2 min from 300° C. to 1070° C., annealing for 5 min, growth for 5 min and finally a quick cooling of the chamber using a water flow (with a decrease rate of 60° C. s−1 from 1000 to 700° C.), for 10 min to reach room temperature.

FET sensor fabrication: Prior to graphene transfer, the interdigitated microelectrodes (ED-IDE1-Au w/o SU8, Micrux Technologies) are cleaned in an UV-Ozone chamber (Jelight, USA) for 10 min followed by submersion for 15 min sequentially in 10 mL of acetone, iso-propanol and water. Finally, every chip is copiously rinsed with large amount of water and dried under a nitrogen flow. The cleaned interfaces are placed in a plastic Petri dish and stored in a desiccator under vacuum. The cleaned IDE are modified with trimethoxyphenylsilane (TMPS, 300 μL of TMPS in 15 mL of ethanol) in a plastic falcon tube for 1 h. Afterwards, the electrodes are immersed for 15 min in ethanol to remove the excess of the silane compound from the surface. Subsequently, the modified interfaces are nitrogen blow-dried and stored under vacuum. The chips are placed on a hot plate at 120° C. at ambient pressure for 1 h to anneal the formed monolayer and provide complete removal of the solvents from the surface. Graphene is directly transferred to these interfaces. For graphene transfer, a polymethyl methacrylate (PMMA) film of 200 nm in thickness is spin-coated onto the graphene/Cu foil and annealed at 110° C. with a very slow heating and cooling rate (1° C. min−1) in order to prevent cracks in the graphene due to the difference of the thermal expansion coefficient between copper and graphene. The graphene on the back side of the Cu foil was re-moved by reactive ion etching (RIE) in an O2 plasma (50 W/100 mT/25 sccm/1 min). Copper foil etching was achieved in 0.2 M ammonium persulfate ((NH4) 2S2O8) for 8 h and the floating PMMA/graphene sample was put in a DI water. This operation was repeated about 10 times in order to rinse the graphene from the etchant solution. Graphene transfer onto the IDE was achieved by submerging the IDE under the floating graphene/PMMA film. To remove traces of trapped water between graphene and IDE and to increase the adhesion of graphene to the IDE, the substrate was placed on a hot plate and annealed at 90° C. for 30 min using a slow heating and cooling rate (1° C. min−1). The PMMA layer was effectively removed by UV/ozone cleaning (28-35 mW cm−2) for 5 min followed by a hot acetone rinse (30° C. for 30 min).

Electrical sensing: Electrical measurements were conducted using a probe station source meter unit U2322A (Keysight Technologies, USA). All measurements were performed using a PMMA commercial flow cell (Micrux Technologies, Spain) with fixed flow channel geometry (16 μL), ensuring a defined flow rate of 50 μL min−1 to minimize mass transport limitation of the analyte to the sensor surface in all experiments. A silver chloride wire (diameter 1 mm, Sigma-Aldrich) was used to operate the GFET device in liquid gate configuration, with a constant gate bias (VGS) of −0.1 V and a constant source-drain bias (VDS) of 0.05 V, sweeping the gate voltage (VG) between −0.8 V and +0.8 V. The general procedure of the sensing experiment started with continuously flushing the pure buffer (PBS, 0.01×) until a stable baseline of drain current was established, followed by injection of the analyte at a constant flow rate of 50 μL min−1.

Researchers have shown an electric field-enhanced electrochemical CRISPR biosensor for DNA detection (Li, et al., Electric field-enhanced electrochemical CRISPR biosensor for DNA detection, Biosensors and Bioelectronics, Volume 192, 2021, 113498, ISSN 0956-5663). A CRISPR electrochemical biosensor was tested to directly detect unamplified human papillomavirus-16 (HPV-16) DNA with a sensitivity of 1 pM with a pulsed electric field used to enrich nucleic acids on the electrode surface.

FIG. 79 is a cross-section view showing a biosensor having an electric field applying structure for concentrating target molecules received at a detection interface, and a local heating element provided at the biosensor for speeding a binding reaction between the capture molecules and the target molecules. The system includes a time release lysing beads 7902, an EBC reservoir 7904, a SAP bead 7906, a wick 7908, a heating element 7910, a biosensor 7912, a capture pool 7914, a top driving electrode 7916, and a bottom driving electrode 7918.

FIG. 80 is an enlarged view showing an electric field that is driving charged target molecules towards capture molecules at a detection interface. The system includes an electric field 8002, a target molecule 8004, a SAP bead 8006, a top driving electrode 8008, and a bottom driving electrode 8010.

The wick includes super absorbent polymer beads having a shell surface, and the electric potential applies a shell charge potential to the shell. The shell charge potential is an opposite charge as a target molecule charge potential and drives the target molecules away from the super absorbent polymer beads while allowing a portion of the fluid sample to enter through the shell and be absorbed by the beads.

FIG. 81 is an isolated view showing SAP beads held on a top driving electrode grid for absorbing excess water from a fluid sample. The system includes a SAP bead 8102, a top driving electrode 8104, and a bottom driving electrode 8106. Alternatively to SAP beads, an SAP powder or SAP/fluid conductive fiber composite material can be provided. The top driving electrode grid applies the electric field while holding back the SAP material from entering into the detection area. Since the wires making up the grid electrode are small, water tension is effective to draw the water in the fluid sample through the grid and to the SAP material. If needed to enhance the concentration of the target molecules in the tested fluid sample, the SAP beads and/or the grid can include a selectively permeable shell, coating or film to block molecules and particles larger than a desired size from being absorbed into the SAP material. In accordance with a non-limiting embodiment, a sensor for detecting target molecules in a fluid sample comprises a detection area for receiving the fluid sample comprising the target molecules and having a detection interface functionalized with capture molecules. At least one of the top and the bottom driving electrode comprises a grid electrode having spaces between conductive elements to allow the fluid sample to flow. A top driving electrode and a bottom driving electrode defining a gap there between. A fluid conductor disposed in the gap conducts the fluid sample through the gap. An electric potential applied to the top and the bottom driving electrode drives the target molecules towards the capture molecules to concentrate the target molecules in a portion of the fluid sample received at the detection interface.

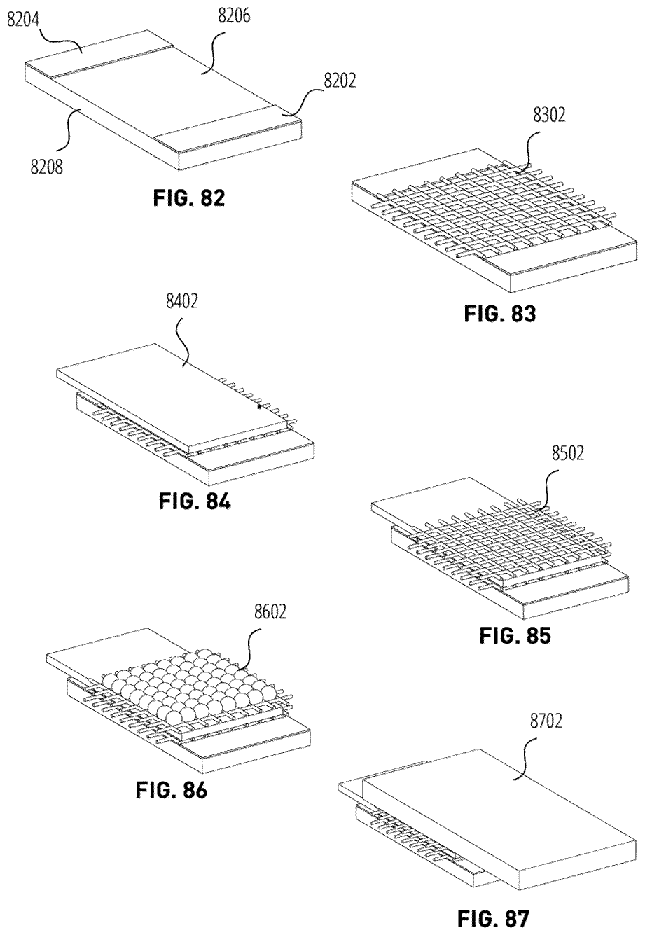

FIG. 82 schematically shows a structure of a field-effect transistor sensor. The system includes a drain 8202, a source 8204, a detection interface 8206, and a substrate 8208.

FIG. 83 schematically shows a structure of the field-effect transistor sensor with electric filed applying bottom driving electrode grid that also acts as a gate electrode for the field-effect transistor sensor. The system includes a bottom driving electrode 8302.

FIG. 84 shows a fluid conductor applied on top of the bottom driving electrode grid/gate electrode. The system includes a fluid transfer 8402.

The fluid transfer 8402, or fluid conductor, comprises at least one of a capillary channel, a microfluidic material, a super absorbent polymer, and a fluid conductive fibrous sheet.

FIG. 85 shows a top driving electrode disposed on the fluid conductor. The system includes a top driving electrode 8502.

FIG. 86 shows SAP beads for removing excess water from the fluid sample held on the top driving electrode grid. The system includes a SAP bead 8602.

FIG. 87 shows a wick in fluid communication with the SAP beads for removing excess water from the fluid sample. The system includes a wick 8702.

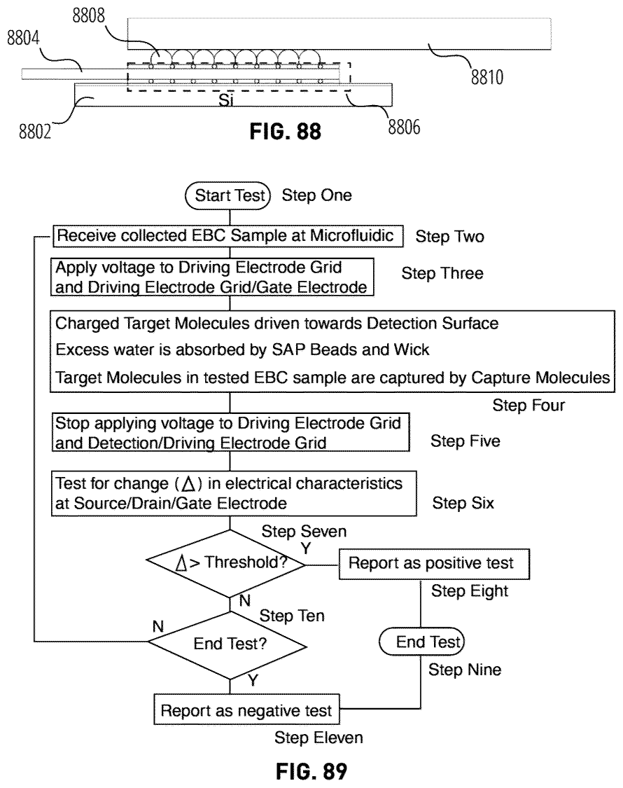

FIG. 88 is a side view of a sensor for detecting target molecules in a fluid sample with a detection area for receiving a fluid sample and a top driving electrode and a bottom driving electrode where an electric potential drives the target molecules towards the capture molecules to concentrate the target molecules in a portion of the fluid sample received at the detection interface. The system includes a substrate 8802, a fluid transfer 8804, a detection interface 8806, a SAP bead 8808, and a wick 8810.

The top and the bottom driving electrode can be disposed in the detection area. A wick absorbs excess fluid from the sample. The fluid conductor conducts a portion of the fluid sample containing relatively less target molecules through the gap to the wick and another portion of the fluid sample containing relatively more target molecules towards the capture molecules. A gate electrode of the sensor comprises at least one of the top and the bottom driving electrode. In the case of the sensor constructed as an electrolyte-gated graphene field-effect transistor, the bottom driving electrode is also the top gate electrode that is in contact with the fluid sample acting as the electrolyte. An applied gate voltage leads to an interfacial charge separation between the electrolyte and the graphene channel, with a diffusive double-layer, serving as a dielectric layer on top of graphene.

FIG. 89 is a flowchart of the steps for concentrating the target molecules in a fluid sample and testing for a change in electrical characteristics of a functionalized transistor sensor.

In accordance with another non-limiting embodiment, a method for detecting a target molecule from a fluid sample comprises the steps of: receiving the fluid sample comprising the target molecule at a microfluidic channel; transferring the fluid sample from the microfluidic channel to a detection interface of a sensor, the sensor comprising a detection area for receiving the fluid sample and having the detection interface functionalized with capture molecules, a top driving electrode and a bottom driving electrode defining a gap there between, and a fluid conductor disposed in the gap for conducting the fluid sample through the gap, wherein an electric potential applied to the top and the bottom driving electrode drives the target molecules towards the capture molecules to concentrate the target molecules in a portion of the fluid sample received at the detection interface, the sensor comprising a field effect transistor having a gate disposed in electrical communication with the detection interface functionalized with the capture molecules, and a source and drain on either side of the gate, and wherein at least a portion of at least one of the top and the bottom driving electrode is disposed at or in electrical communication with the detection area, and where a gate electrode of the sensor comprises at least one of the top and the bottom driving electrode; and intermittently and selectively applying electric potentials to driving electrodes and gate, source and drain electrodes for driving the target molecules and for taking a test reading of a change in an electrical characteristic at the source, drain and gate.

As shown in FIG. 89, at the start of the test (step one) a collected EBC sample is received at the microfluidics (step two). A voltage is applied to the driving electrode grid and the driving electrode grid/gate electrode (step three). The applied voltage drives the charged target molecules in the EBC sample towards the capture molecules at the detection interface of the biosensor to concentrate the target molecules in a portion of the fluid sample received at the detection interface, excess water is absorbed by SAP and/or the wick, and the target molecules in the tested EBC sample are captured by the capture molecules immobilized at the detection interface. Applying the electric potential for driving the target molecules is stopped (step five). A test reading taken of a change in an electrical characteristic caused by the captured target molecules affecting the electron charge mobility at the detection interface (e.g., through the charge interactions of the target molecule and capture molecule at a distance that is less than the Debye screening length for the capture molecule immobilized on the graphene layer shown in FIG. 78). Stated otherwise, the electric potential for driving the target molecules is applied intermittently with taking a test reading of a change in an electrical characteristic at the source/drain/gate electrodes of the transistor biosensor (step six). Alternative electrode and biosensor configurations can also be used, including printed electrodes with nanoparticle, nanotube, metal, semiconductor and/or organic detecting interface materials. If the change in electrical characteristics is greater than a threshold value (step seven) then a positive test is reported (step eight) and the tested ended (step nine). If the change is less than the threshold value (step seven) then it is determined if it is time to end the test (step ten). For example, the test can be ended after a given period of time or a given amount of fluid sample is tested. If it is not time to end the test (step ten) then the process flow returns to receiving more of the collected EBC sample at the microfluidic (step two). If it is time to end the test (step ten) and the change in electrical characteristics has not exceeded the threshold (step seven), then a negative test is reported (step eleven) and the test is ended (step nine).

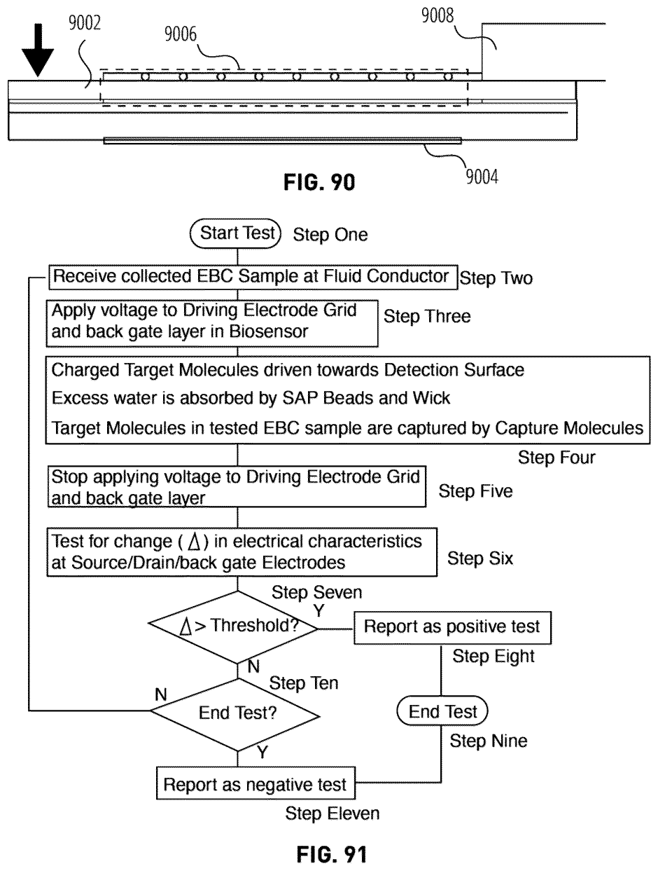

FIG. 90 is a side view of a back gated field-effect transistor sensor with a detection area for receiving a fluid sample and a top driving electrode working in cooperation with a back gate electrode for applying an electric potential that drives the target molecules towards the capture molecules to concentrate the target molecules in a portion of the fluid sample received at the detection interface. The system includes a fluid transfer 9002, a back gate 9004, a detection interface 9006, and a wick 9008.

The sensor comprises a field-effect transistor having a gate disposed in electrical communication with the detection interface functionalized with the capture molecules, and a source and drain on either side of the gate. At least a portion of at least one of the top and the bottom driving electrode is disposed in electrical communication with the detection area, and where a gate electrode of the sensor comprises at least one of the top and the bottom driving electrode. In the case of the sensor constructed as a back gated field-effect transistor, the bottom driving electrode can also be the back gate electrode of the transistor. When an electric potential is applied to the top driving electrode grid and the back gate electrode, target molecules having a charge are driven towards the detection interface. For example, if the target molecule is the S-protein of the SARS-CoV-2 virus, the target molecule has a negative charge. In this case, the top driving electrode grid is applied with a negative potential and the back gate electrode is provided with a positive potential to drive the negatively charged S-protein target molecule towards the capture molecules immobilized on the detection interface.

FIG. 91 is a flowchart of the steps for using an applied electric field for concentrating target molecules and testing for a change in electrical characteristics of a functionalized field-effect transistor sensor. Similar to the flowchart shown in FIG. 89, at the start of the test (step one) a collected EBC sample is received at the microfluidics (step two). In the case of a back gated transistor biosensor, the back gate of the transistor can be utilized as both the bottom driving electrode for driving the target molecules towards the detection interface and the gate electrode for testing for captured target molecules by capture molecules immobilized at the detection interface. The voltage is applied to the driving electrode grid and the back gate layer of the biosensor (step three). The applied voltage drives the charged target molecules in the EBC sample towards the capture molecules at the detection interface of the biosensor to concentrate the target molecules in a portion of the fluid sample received at the detection interface, excess water is absorbed by SAP and/or the wick, and the target molecules in the tested EBC sample are captured by the capture molecules immobilized at the detection interface. Applying the electric potential for driving the target molecules is stopped (step five). A test reading taken of a change in an electrical characteristic at the source/drain/back gate layers/electrodes of the transistor biosensor (step six). If the change is greater than a threshold value (step seven) then a positive test is reported (step eight) and the tested ended (step nine). If the change is less than the threshold value (step seven) and if it is not time to end the test (step ten) then the flow returns to receiving more of the collected EBC sample at the microfluidic (step two). If it is time to end the test (step ten) and the change in electrical characteristics has not exceeded the threshold (step seven), then a negative test is reported (step eleven) and the test is ended (step nine).

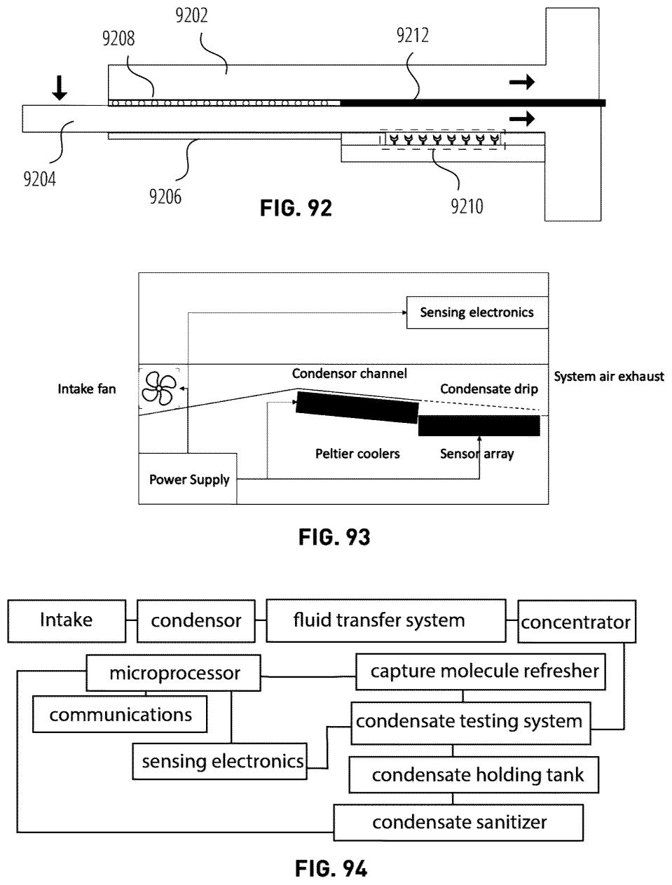

FIG. 92 is a side view of a sensor for detecting target molecules in a fluid sample with a detection area for receiving a portion of the fluid sample downstream from where a top driving electrode and a bottom driving electrode apply an electric potential to separate the target molecules from excess water in the fluid sample. The system includes a wick 9202, a fluid transfer 9204, a bottom driving electrode 9206, a top driving electrode 9208, a detection interface 9210, and a separator 9212.

In accordance with this embodiment, the top and the bottom driving electrode can be disposed upstream of the fluid sample flow from the detection area. The fluid conductor is provided upstream and downstream from the detection area to flow the fluid sample with the target molecules over the detection area. A wick removes excess water from the fluid sample upstream from the detection area, with a separator keeping the flow of excess water in the wick separate from the flow of the tested sample with the target molecules through the fluid conductor. The fluid conductor can have a different composition at different locations in the fluid flow path, for example, a hydrophilic surface can be provided at the EBC collector condensing surface, a fibrous microfluidic material can be provided for drawing the collected EBC to the detection area, and to maximize the particle and ion mobility for alignment and movement at the detection area, a capillary channel can be provided where the inherent surface tension of the water in the fluid sample acts to draw the fluid sample over and past the detection interface. A fibrous wick, SAP composite or other absorbing material can be provided downstream from the detection area.

FIG. 93 schematically shows a room scale biosensor for detecting target molecules in a fluid sample comprised from ambient humidity pulled from the room.

FIG. 94 is a block diagram showing the components of the room scale biosensor.

In accordance with a non-limiting exemplary embodiment, a room scale biosensor comprises an intake for taking in ambient air. A condenser cools the ambient air to condense moisture vapor to a condensate containing water and at least one target molecule. A condensate testing system tests the condensate for the target molecule. The condensate testing system includes a biosensor comprising one or more g-FET biosensors each having a detection interface comprising a graphene layer functionalized with respective capture molecules. In a preferred embodiment, the capture molecules are smaller than the Debye screening length for the fluid sample.

A target molecule concentrator can be provided for concentrating the at least one target molecule in the fluid biosample to form a target molecule concentrated condensate. The target molecule concentrator may comprise a selectively permeable barrier for allowing excess water in the condensate to pass and block the target molecule in the condensate from passing through the selectively permeable barrier, and an excess water absorbing wick for absorbing the excess water passing through the selectively permeable material.

The target molecule concentrator can also or alternatively comprise a super absorbent polymer (SAP) for preferentially absorbing water from the condensate into polymer chains of the super absorbent polymer, wherein the target molecule is not absorbed by the polymer chains and flows along with the condensate through the SAP. As the condensate flows along through the SAP, the water content in the condensate is removed to increase the tested sample concentration of the target molecules.

The biosensor can include an array of respective biosensors, each functionalized with different corresponding capture molecules designed to bind to at least one biomarker of FluA, FluB, SARS (SARS N-protein and SARS S-protein). The biosensor can also include capture molecules to test for environmental exposures, VOCs, bomb making materials, and other markers that may be present in the sampled ambient air.

A fluid transfer system can be provided for transferring the condensate from the condenser to the condensate testing system where the fluid transfer system includes at least one of a fluid conductor, a pooling area, and a microfluidics transfer path for controlling a flow of the fluid biosample received from the EBC collector. A target molecule releasing material can be disposed in the fluid conductor, pooling area and microfluidics transfer path, or anywhere along the path from the condenser to the biosensor, where the target molecule releasing material includes at least one of a surfactant and a chemical lysing agent.

In addition or alternatively to the surfactant and/or chemical lysing agent, a target molecule releasing structure can be provided for mechanically lysing at least one of a cell wall, encapsulating structure and viral envelope containing the target molecule. The target molecule releasing structure can comprise lysing structures protruding from at least one of the condensate-forming surface and the surface of a flow path of the EBC. The lysing structures mechanically disrupt the cell wall, encapsulating structure and/or vial envelope containing the target molecule.

The condenser can also include a condensate-forming surface comprising a relatively low energy surface property for limiting an adhesion of target molecule to the condensate-forming surface. A fluid conductor can be disposed on the condensate-forming surface, where the fluid conductor comprises a textured structure formed on the condensate-forming surface with the textured structure having a relatively higher energy surface property for guiding a flow of the EBC towards a desired direction.

In operation, an example embodiment of the room scale biosensor is placed in a room, or within the HVAC system of a building. An intake fan draws ambient air in and over to a condenser channel that is cooled by a cooling system, such as coils with a working fluid (e.g., a conventional dehumidifier or air conditioner) or by another means such as Peltier coolers. Humidity in the cooled air is condensed into a fluid sample condensate that contains particles, dissolved VOCs, non-target molecules and target molecules, etc. The condensate is transferred to a target molecule concentrator via a fluid transfer system (capillary action, surface flow, wick, etc.). The target molecule concentrated condensate drips onto or is otherwise received at a condensate testing system containing at least one biosensor. The condensate can be flowed over the biosensor for testing then transferred to a condensate holding tank, or the sensor can be located in the condensate holding tank. Sensing electronics receive a test signal from the condensate testing system and determine if a target molecule has been detected. The sensing electronics can determine the presence and/or the concentration of the target molecule present in the ambient air. A microprocessor controls the operation of the constituent parts of the room scale biosensor and receives a test result signal from the sensing electronics. If a target molecule is detected, the microprocessor can control a condensate sanitizer to automatically inject a sanitizing solution into the condensate holding tank and/or through the entire system. A capture molecule refresher system can be controlled to wash a captured molecule release agent, such as propylene glycol or glycerol, over the detection interface to refresh the biosensor array. A communications system, which may include at least some of the components shown and described herein, for example at FIGS. 3-6, the communications system is controlled by the microprocessor to automatically send out an alert or report, which may include details on the type(s) of target molecule detected, the time of detection and concentration.

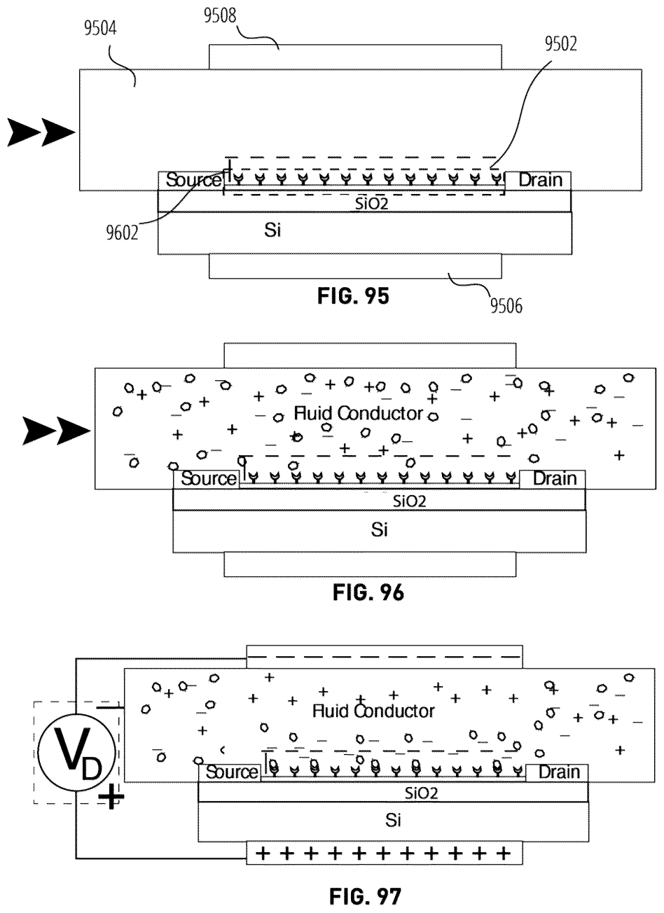

FIG. 95 is an illustration of a field-effect transistor biosensor with a fluid sample flow through conductor and capture molecules standing off from the conductive surface of a detection interface at a height less than the Debye screening length for the fluid sample being tested. The system includes a detection interface 9502, a fluid transfer 9504, a back gate 9506, and a liquid gate electrode 9508.

In accordance with a non-limiting exemplary embodiment, a field-effect transistor sensor circuit for detecting target molecules in a fluid sample comprises a semiconductor substrate of one conductivity type. A source region and a drain region are provided defining there between a channel region of the one conductivity type. The source region and the drain region are of an opposite conductivity type to the semiconductor substrate (e.g, a PNP or NPN transistor construction). An insulator is formed over the channel region and the channel region forms a back gate of the field-effect transistor. A detection area is formed over the insulator for receiving the fluid sample and has a detection interface functionalized with capture molecules.

FIG. 96 is an illustration of the field-effect transistor biosensor showing a fluid sample flow with randomly dispersed target molecules and ions in a flow conductor above the detection interface. The system includes a Debye screening length 9602. A top electrode defines a gap with the detection area and forms a liquid gate electrode of the transistor.

A fluid conductor is disposed in the gap for conducting the fluid sample through the gap. The fluid conductor can be formed, for example, from one or more components in fluid communication with each other. For example, the fluid conductor can be made from a microfluidic material, a capillary channel (thin empty space for the fluid sample to flow via capillary action), a fibrous wick, micro-channels, etc.

FIG. 97 is an illustration of a field-effect transistor sensor sensing circuit for detecting target molecules in a fluid sample showing a driving circuit for driving the target molecules towards the capture molecules immobilized on the detection interface.

As an example, the electronics for the driving circuit and for reading the test signal can include a battery power source and a Synchronous Step-Up DC/DC Converter, such as the XC series of discrete electronic components made by Torex Semiconductor, LTD. The DC/DC Converter can be used in series as necessary to step up the battery voltage so that it is useful for aligning and driving the target molecules towards the detection interface. The stepped-up voltage can also be used in the case of a biosensor construction that has a back gate disposed under an insulating layer on which is formed the detection interface. Most or all of the discrete semiconductor devices making up the driving circuit, the detection circuit and the biosensor can be fabricated as an integrated circuit on a semiconductor wafer significantly lowering the cost, power requirements and complexity while raising the consistency and accuracy of the system. The driving circuit applies an electric potential of one polarity to the top electrode and of the opposite polarity to the back gate. The electric potential drives the target molecules towards the capture molecules to concentrate the target molecules in a portion of the fluid sample received at the detection interface. The detection interface comprises at least one of graphene, nanoparticles, aligned carbon nanotubes, vacuum deposited conductive material, transferred conductive material, a semiconductor, a metal, and a screen-printed conductive ink.

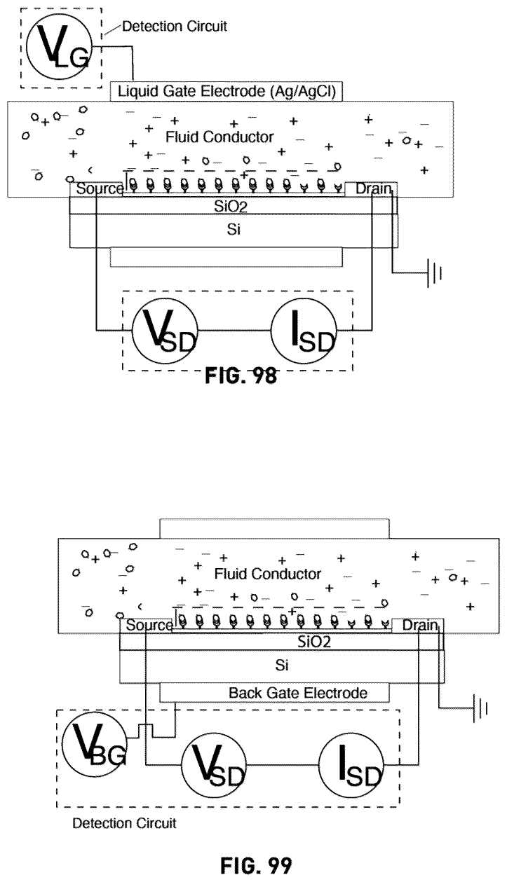

FIG. 98 is an illustration of a field-effect transistor sensor sensing circuit for detecting target molecules in a fluid sample showing a liquid gate detecting circuit for detecting the presence and quantity of target molecules captured at the detection interface.

FIG. 99 is an illustration of a field-effect transistor sensor sensing circuit for detecting target molecules in a fluid sample showing a back gate detecting circuit for detecting the presence and quantity of target molecules captured at the detection interface. The detection circuit applies a voltage to the source region and at least one of the back gate and the top electrode, and detects a change in current through the drain region dependent on a binding of the target molecules with the capture molecules. The driving circuit and the detection circuit can be controlled to operate cyclically in sequence with each other, and may include a test reading taken using both the back gate and the liquid gate.

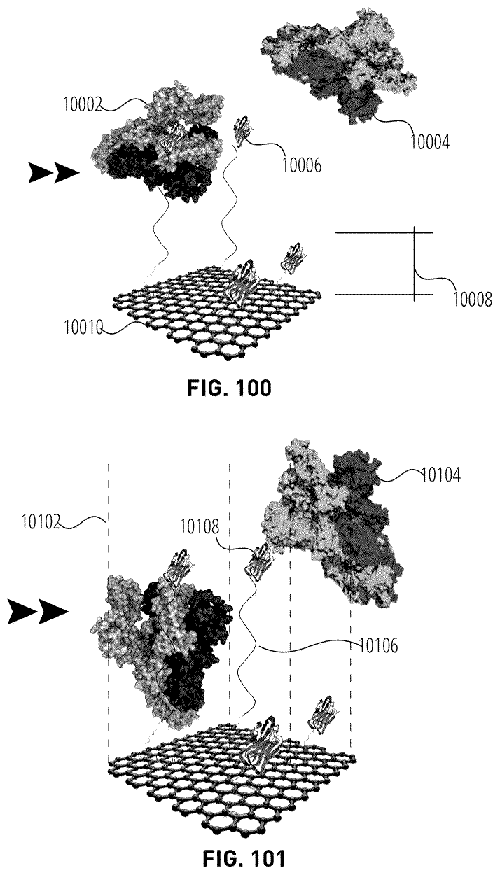

FIG. 100 illustrates a graphene detection interface with nanoCLAMP capture molecules immobilized by linker molecules, with a portion of the capture molecules immobilized at a greater distance than another portion of the capture molecules immobilized on the detection interface. The system includes a non-target protein 10002, a target protein 10004, a nanoCLAMP 10006, a Debye screening length 10008, and a graphene 10010.

FIG. 101 illustrates an electric field potential applied in the detection area and driving a target molecule and non-target molecule towards the capture molecules, where the target molecule is captured by a capture molecule extending on linker molecules a relatively longer distance from the detection interface. The system includes an electric field 10102, a target protein 10104, a linker 10106, and a capture molecule 10108.

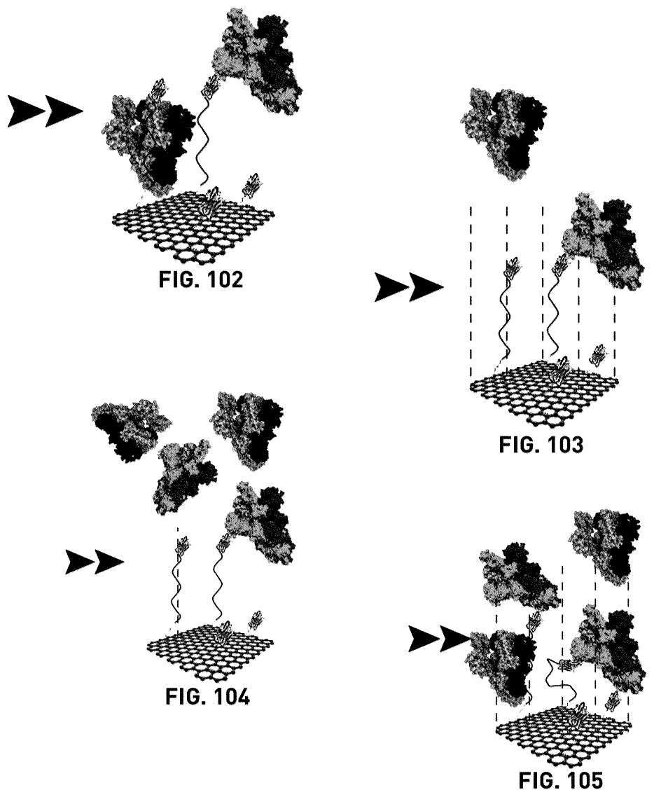

FIG. 102 illustrates the electric field potential removed from detection area. In accordance with a non-limiting embodiment, a portion of the capture molecules is immobilized on the detection interface at a greater distance from a top surface of the detection interface than another portion of the capture molecules immobilized on the detection interface. As a target molecule is driven towards the detection interface, it may encounter a capture molecule tethered at a relatively longer stand-off distance and get captured and thus immobilized and tethered to the detection interface.

FIG. 103 illustrates an opposite polarity electric field potential that drives the target molecule and non-target molecule away from the detection interface.

The target molecule remains tethered by the capture molecule immobilized by the relatively longer linker on the detection interface. The non-target molecules and ions contained in the fluid sample along with the target molecule that are not captured are driven further away from the detection interface by the opposite polarity electric field. The driving circuit reverses polarity of the applied electric potential to drive non-target molecules from the detection area (making room for another target molecule to migrate towards the detection interface) while target molecule captured by capture molecules immobilized on the detection interface are retained in the detection area.

FIG. 104 illustrates more target molecules and non-target molecules flowing into the detection area.

FIG. 105 illustrates the electric field potential applied in the detection area, driving the tethered target molecule into position to be captured by a capture molecule immobilized by a relatively shorter linker molecule. Also, another target molecule is captured by another capture molecule extending on linker molecules a relatively longer distance from the detection interface.

This cycle of reversing electric field polarity can be used to concentrate the target molecules in a portion of the fluid sample received at the detection interface. Some target molecules may get captured directly at the shorter standoff distance capture molecules, or through a pumping action of cycling the polarity of the applied electric field, the target molecules over time become more prevalent and captured by the capture molecules at the detection interface while the non-target molecules and ions present at the detection interface are reduced. Through the application of the electric field and the tethering and selective binding by the capture molecules bound to target molecules within the Debye screening length, the resulting different capture molecule standoff distances from the detection surface produce different sensor-to-antigen binding site distances with the capture molecules that bind to the target molecules within the Debye screening length contributing a stronger signal response of the detection circuit.

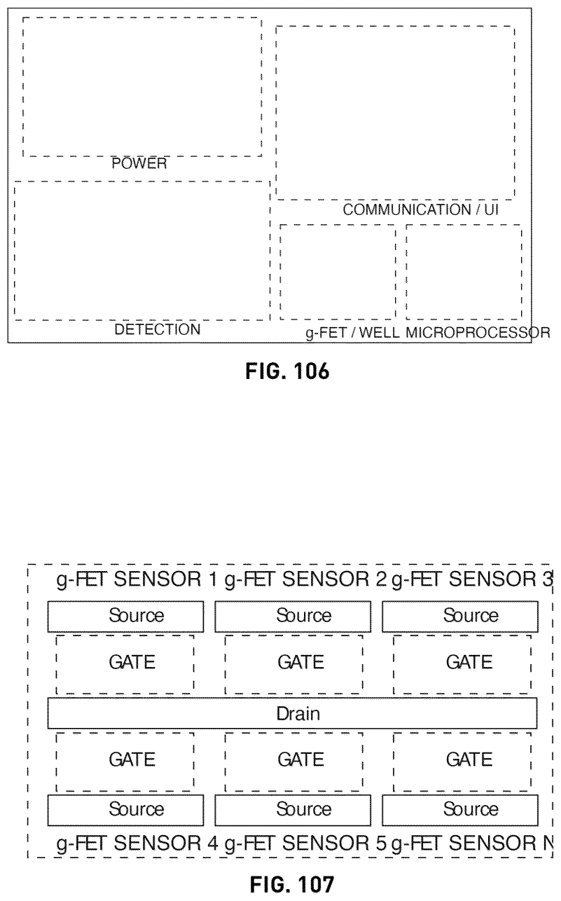

FIG. 106 is a block diagram of an g-FET sensor integrated circuit having a functionalizable detection interface. An integrated circuit can be provided that includes a packaged biosensor semiconductor device with access for a fluid sample to a detection interface of the biosensor. For example, a universal biosensor IC chip can include most if not all the electronic circuit elements formed through conventional IC fabrication techniques, with the additional feature of providing access to detection interface. In an example universal biosensor that can detect target RNA and DNA molecules, a Cas9 complex is immobilized on a graphene detection interface. The adsorption and interaction of charged molecules at the surface of the graphene detection interface causes a change in electrical characteristics among the source, drain and gate(s) electrodes. When a detection circuit applies a voltage across the surface of the detection interface, the capturing of target DNA molecules by the immobilized ribonucleoproteins (CRISPR Cas protein capture molecule complexes) is detectable as a change in electrical current and transmitted as a direct-to-electrical signal directly from a packaged integrated circuit semiconductor device. Just a few leads are needed, for connecting to an external power source and antenna. In the case of a near-field-communications and energy harvesting scenario, the only leads needed may be for connecting with an RF energy harvesting and RF transmitting antenna.

FIG. 107 shows ganged g-FET sensors fabricated in a functionalizable detection area comprising individually addressable channel regions. A multi-biomarker, syndromic testing system can be obtained through a ganged biosensor array fabricated at the wafer level and that receives a collected EBC biosample from a mask-based diagnostic system. This ganged biosensor has different channel areas functionalized for a specific use-case through the selection of a respective capture molecule for a corresponding target molecule.

FIG. 108 shows a g-FET semiconductor device encapsulated in an epoxy base with the detection interface of the g-FET exposed at the surface of the epoxy base. The system includes a detection interface 10802, a drain 10804, a source 10806, and a detection interface 10808.

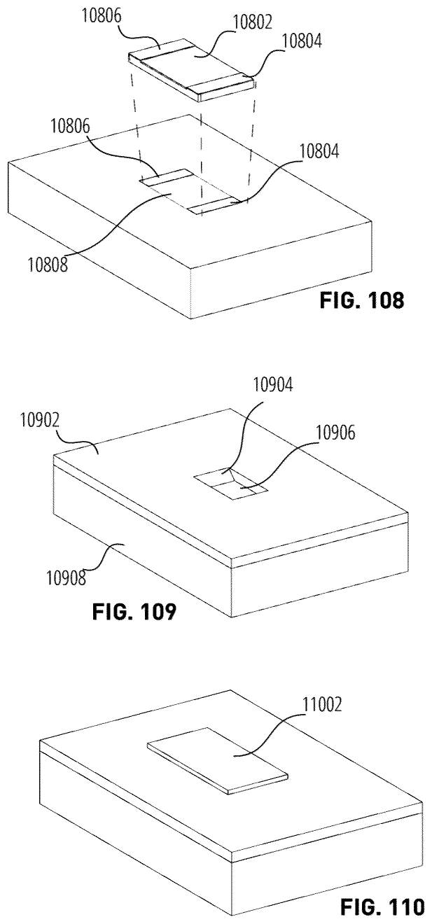

FIG. 109 shows an epoxy top with a detection well opening disposed over the epoxy base with the detection interface accessible through the detection well opening. The system includes an epoxy top 10902, a detection well 10904, a detection interface 10906, and an epoxy base 10908.

FIG. 110 shows a fluid conductor in fluid communication with the detection interface through the detection well. The system includes a fluid transfer 11002.

FIG. 111 shows a mesh driving electrode/liquid gate electrode disposed on the fluid conductor. The system includes a liquid gate electrode 11102.

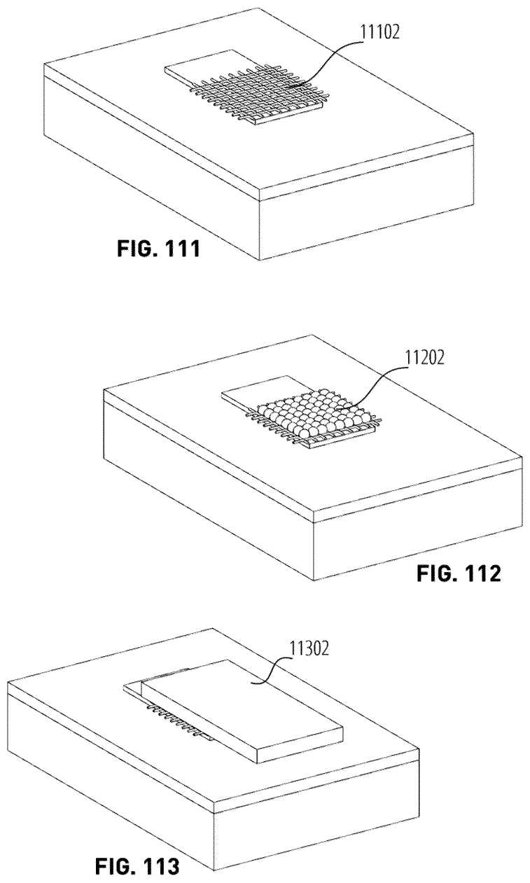

FIG. 112 shows SAP optionally provided on the top of the mesh driving electrode/liquid gate electrode. The system includes SAP beads 11202.

FIG. 113 shows a wick disposed over the SAP and the mesh driving electrode/liquid gate electrode. The system includes a wick 11302.

In accordance with a non-limiting embodiment, the capture molecules include at least one of antibodies, engineered antibodies, aptamers, nanoCLAMPS, and CRISPR Cas protein conjugates.

CRISPR-associated (Cas) proteins (e.g., Cas9, Cas12a, Cas13a) have recently been used for sequence-specific nucleic acid detection. In a specific construction of a non-limiting exemplary embodiment, the field-effect transistor sensor is a g-FET with monolayer graphene sheet disposed as the detection interface. The capture molecules includes a catalytically inactive Cas9 protein conjugated with guide RNA and immobilized on the graphene detection interface. The Cas9 protein complex is provided as the capture molecule to form a CRISPR universal biosensor. The Cas9 capture molecule binds with a target DNA molecule, causing a detectable change in electrical characteristics at the biosensor electrodes.

The CRISPR cleavage (gene editing) capability may be used to detect a suppressed current flow when a target molecule is captured (Wei Xu, Tian Jin, Yifan Dai, Chung Chiun Liu, Surpassing the detection limit and accuracy of the electrochemical DNA sensor through the application of CRISPR Cas systems, Biosensors and Bioelectronics, Volume 155, 2020, 112100, ISSN 0956-5663).

CRISPR technology can be used to improve the sensitivity, selectivity, and specificity of the biosensors disclosed herein while simplifying the detection process. Because of the long distance between linear ssDNA and the electrodes including the detection interface, the efficiency of electron transfer will be affected, and a certain steric hindrance will be generated, which affects the cutting of linear DNA by Cas12a. To overcome this problem, hairpin ssDNA with two ends connected to the detection interface and a receptor like methylene blue (MB) respectively, directly improves the analytical performance of the biosensor (g-FET, electrochemical, etc). Depending on the length and amount of DNA fragment on the Cas hairpin form the ss DNA is the same size as nanoCLAMP (but it depends on the length and amount of DNA fragments on it).

Alternatively, researchers have described a g-FET CRISPR universal biosensor where the Cas9 protein is conjugated with guide RNA and immobilized on a graphene layer at the detection interface (https://www.synthego.com/blog/crispr-electronic-biosensor). The catalytically inactive Cas9 protein conjugate recognizes and binds to the target DNA molecule but does not cleave the DNA. The target DNA is tethered to the detection interface. The catalytically inactive Cas9 protein can be immobilized on graphene using PBA linker and is smaller than the Debye screening length for detecting a target DNA or RNA molecule in an exhaled breath condensate biosample.

FIG. 114 shows a packaged g-FET semiconductor device encapsulated in an epoxy barrier material with source, gate and drain leads. The system includes a detection well 11402, a drain 11404, a gate 11406, and a source 11408.

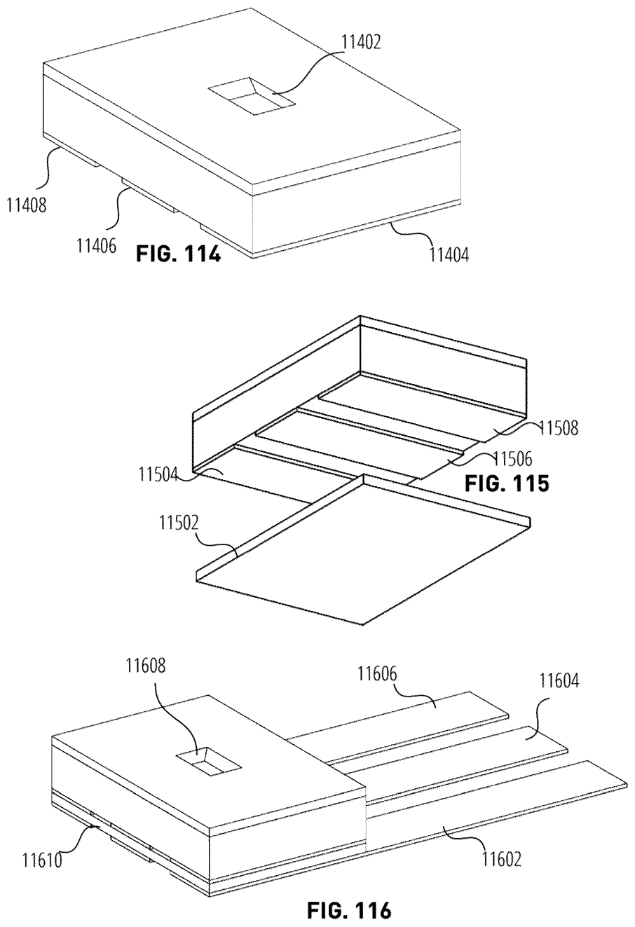

FIG. 115 shows a z-axis conductor for connecting the source, gate and drain leads of the packaged g-FET semiconductor device to an external electrical circuit. The system includes z-axis conductive tape 11502, a source 11504, a gate 11506, and a drain 11508.

FIG. 116 shows the packaged g-FET semiconductor device connected to source, gate and drain circuit lines of an external electrical circuit. The system includes a drain 11602, a gate 11604, a source 11606, a detection well 11608, and z-axis conductive tape 11610.

A double-sided sticky z-axis tape (e.g., 3M 9703, 3M, Minnesota) can be used to make simple and effective electrical connections between the leads of the packaged IC semiconductor device and the corresponding lead lines and connection pads of the detection circuit, power, or other external electrical circuits. The z-axis tape also provides the potential for manually placing the packaged IC semiconductor device making it easy for an end-user to select for the detection of target biomarker(s) depending on a diagnostic use-case.

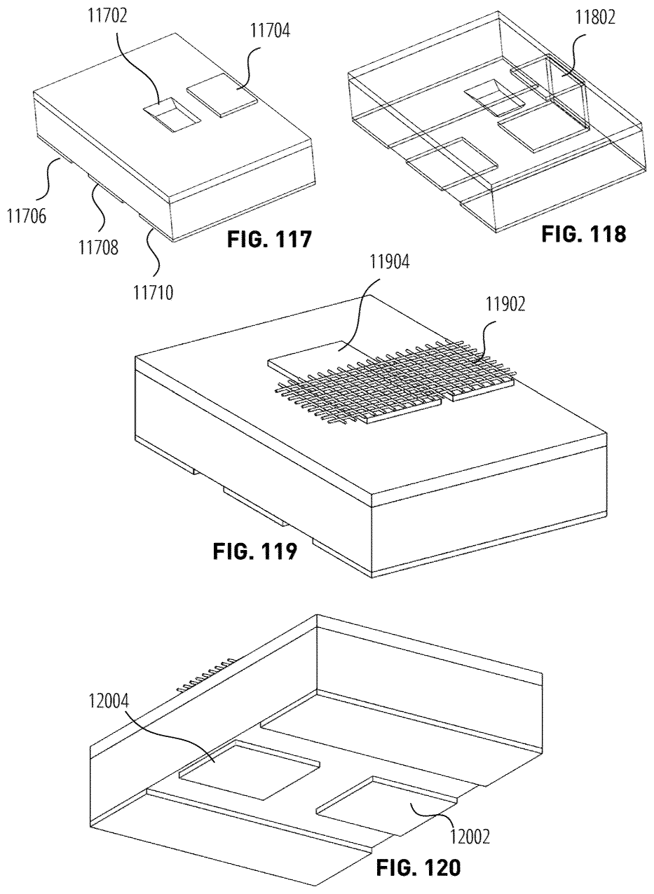

FIG. 117 shows the packaged g-FET semiconductor device having a driving electrode/liquid gate lead disposed for connecting the mesh driving electrode/liquid gate electrode to a lead line of an external electrical circuit. The system includes a detection well 11702, a liquid gate electrode 11704, a source 11706, a back gate 11708, and a drain 11710.

The packaged biosensor semiconductor device comprises a semiconductor die mounted on a lead frame and encapsulated in a barrier material, such as epoxy. The semiconductor die includes a source region and a drain region defining there between a channel region. An insulator is formed over the channel region. The channel region forms a back gate of the field-effect transistor. A detection area is disposed over the insulator and receives the fluid sample. The detection area has a detection interface that is functionalized with capture molecules.

The capture molecules can be mobilized at the wafer fabrication layer through a conventional masking/etching semiconductor fabrication process. At the near final steps in the fabrication process, the detection interface is applied to at least one channel region, for example the vacuum deposition, transfer, spin coating, or other deposition methods. Different capture molecules can be selectively applied at the channels through masks and etching. At least the final mask applied over the detection region as the detection interface is formed is a thin water soluble layer that allows for the preservation of the graphene detection interface decorated with immobilized capture molecules. Spacer molecules, such as PEG, can also be immobilized to create a desired density of capture molecules on the detection interface. The water soluble mask layer protects the detection interface through die-on-wafer testing, singulation, sorting, packaging, etc. Proper solvents, handling and materials need to be used during washing and other processes to prevent the water soluble mask layer from pre-maturely dissolving.

The singulated die is mounted on a die pad that has leads for conducting electrical signals from the source, channel and drain regions to an external electronic circuit. A base barrier section (epoxy base) encapsulates the semiconductor substrate and leaves the detection interface exposed. A top barrier section (epoxy top) has a detection well forming an opening over the exposed detection interface for receiving a fluid sample (where the floor of the well is the detection interface decorated with capture molecules).

A top gate lead is provided with one end at the top surface of the top barrier section. The top gate lead connects to the liquid gate electrode. Another end of the top gate lead connects the liquid gate electrode to the external electronic circuit. The liquid gate electrode defines a gap with the detection region and is in electrical communication with a fluid sample when the fluid sample is disposed in the detection well. The geometry and volume of the detection well accommodates and optimizes the collected biosample. For example, in an experimental test, EBC was successfully tested as a unaltered biosample flowing through a fibrous microfluidic material (filter paper) in contact with the detection interface of an electrochemical biosensor with aptamer capture molecules. Also, or alternatively, a capillary channel can be formed with the detection well. A combination of microfluidic materials and structures can be provided to flow the sample from collection through to a final wicking stage.

The target molecules in the fluid sample are detected by a change in electrical characteristics occurring at one or more of the source, drain, back gate and liquid gate electrode when the capture molecules bind to the target molecules. Typically, the change in the detected signal is proportion to the number of target molecules bound and held by the capture molecules. The capture molecules can include antibodies, engineered antibodies, aptamers, nanoCLAMPS, and/or CRISPR Cas protein conjugates.

FIG. 118 is a wireframe of the packaged g-FET semiconductor device showing the driving electrode/liquid gate lead disposed on the top of the epoxy top and at the bottom of the epoxy base for connecting the mesh driving electrode/liquid gate electrode to a lead line of an external electrical circuit. The system includes a liquid gate electrode 11802.

The driving electrode/liquid gate lead disposed on the top of the epoxy top can be connected to a detection circuit integrated into an IC package. A packaged semiconductor device that includes a universal biosensor is provided from a g-FET die singulated from a wafer and encapsulated in epoxy (typical semi-conductor discrete electronic device construction). A detection well is formed in molded epoxy top and disposed over the graphene detection interface so the fluid sample can reach the immobilized capture molecules at the detection interface. The graphene layer may be formed and functionalized with the capture molecules at the wafer level. During fabrication, the functionalized graphene is protected with a dissolvable mask left in place so the die can be picked and placed, and mounted on a lead frame then encapsulated in epoxy (with the detection well either preformed in an epoxy cap or formed in place during encapsulation).

FIG. 119 shows the packaged g-FET semiconductor device having the driving electrode/liquid gate lead disposed on top of the fluid conductor for connecting to a lead line of an external electrical circuit. The system includes a top driving electrode 11902 and a fluid transfer 11904.

FIG. 120 shows the bottom of the packaged g-FET semiconductor device showing the source and drain leads, and the back gate and driving electrode/liquid gate leads. The system includes a back gate 12002 and a liquid gate electrode 12004.

The packaged biosensor semiconductor device can include both a liquid gate and/or back gate optional connection, which can be mounted and externally connected to an electric circuit on a printed circuit board. The liquid gate, back gate, source and drain connection can be made to other constituent semiconductor circuits on the same packaged IC device. The liquid gate may be formed from a mesh electrode that allows for the removal of excess water in the fluid sample just before the sample reaches the detection well so a concentrated capture molecule sample continuously flows into the well as the fluid sample accumulates.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1. An apparatus, comprising:
an exhaled breath condensate (EBC) collector for converting breath vapor received from the lungs and airways of a test subject into an EBC fluid biosample;
a biomarker concentrator for concentrating a target biomarker portion in the fluid biosample to form a concentrated fluid biosample;
a biomarker testing unit for receiving the concentrated fluid biosample and testing the concentrated fluid biosample for a target biomarker.

2. The apparatus of claim 1, further comprising a testing system support for the EBC collector, wherein the testing system support is configured and dimensioned to fit inside a face mask, wherein the face mask forms an exhaled breath vapor containment volume to hold the exhaled breath vapor in proximity to the EBC collector to enable the exhaled breath vapor to coalesce into the fluid biosample.

3. The apparatus of claim 1, wherein the biomarker concentrator comprises a selectively permeable barrier for allowing excess water in the fluid biosample to pass through the selectively permeable barrier and block the target biomarker in the fluid biosample from passing through the selectively permeable barrier.

4. The apparatus of claim 3, further comprising an excess water absorbing wick for absorbing the excess water passing through the selectively permeable material.

5. The apparatus of claim 1, wherein the biomarker concentrator comprises a super absorbent polymer for preferentially absorbing water from the EBC into polymer chains of the super absorbent polymer, wherein the target biomarker is not absorbed by the polymer chains and flows along with the EBC through the SAP and microfluidics structures of the diagnostic platform, wherein as the EBC flows along through the SAP the water content in the EBC is removed while the content of the target molecules remains constant, increasing the tested sample concentration of the target molecules.

6. The apparatus of claim 1, further comprising applied-field-reactive capture molecule conjugate provided for capturing the target biomarker, the applied-field-reactive capture molecule conjugate having at least one applied-field-responsive end and a capture molecule end, wherein the capture molecule end binds to and capture the target biomarker.

7. The apparatus of claim 6, further comprising a dissolvable adhesive for holding the applied-field-reactive capture molecule conjugate in a path of the fluid biosample, wherein water in the fluid biosample dissolves the dissolvable adhesive and allows the applied-field-reactive capture molecule conjugate to be free floating in the fluid biosample.

8-10. (canceled)

11. The apparatus of claim 6, wherein the applied-field-reactive capture molecule conjugate comprises a linker molecule disposed between the applied-field-responsive end and the capture molecule end, the linker molecule providing the applied-field-reactive capture molecule conjugate with electro-chemical properties wherein when the capture molecule end binds with the target biomarker at least one of a polarity and a conductivity of the applied-field-reactive capture molecule conjugate changes.

12. (canceled)

13. A method for assembling an array of applied-field-reactive capture molecule conjugates, comprising: providing dissolvable adhesive film; providing a carrier fluid that is a non-solvent for the dissolvable adhesive film, the carrier fluid having randomly dispersed applied-field-reactive capture molecule conjugates; applying an aligning field to the carrier fluid for assembling the applied-field-reactive capture molecule conjugates onto the dissolvable adhesive film; and evaporating the carrier fluid leaving the assembled applied-field-reactive capture molecule conjugates fixed on the dissolvable adhesive film.

14. An exhaled breath condensate (EBC) collector for converting breath vapor received from the lungs and airways of the test subject into a fluid biosample, the EBC collector including: a condensate-forming surface; and a thermal mass in thermal connection with the condensate-forming surface; and
a fluid transfer system for transferring the EBC to at least one of a testing unit and an EBC containment vessel.

15. The EBC collector of claim 14, wherein the thermal mass comprises at least a first chemical reagent and a second chemical reagent combinable to form an endothermic chemical reaction for absorbing thermal energy from the condensate-forming surface for converting the exhaled breath vapor to the EBC.

16. The EBC collector of claim 14, wherein the fluid transfer system includes at least one of a fluid conductor, a pooling area, and a microfluidics transfer path for controlling a flow of the fluid biosample received from the EBC collector; and further comprising a target biomarker releasing material disposed in said at least one of the fluid conductor, pooling area and microfluidics transfer path.

17. The EBC collector of claim 16, wherein the target biomarker releasing material includes at least one of a surfactant and a chemical lysing agent.

18. The EBC collector of claim 17, wherein the condensate-forming surface comprises a relatively low energy surface property for limiting an adhesion of target biomarker to the condensate-forming surface, and further comprising a fluid conductor disposed on the condensate-forming surface, wherein the fluid conductor comprises a textured structure formed on the condensate-forming surface, the textured structure having a relatively higher energy surface property for guiding a flow of the EBC towards a desired direction.

19-20. (canceled)

21. A method for forming a condensate collector having fluid conductor channels on a substrate for guiding a flow of fluid towards a desired direction, comprising:
providing the substrate having a surface having a relatively lower energy surface property; forming a textured structure forming fluid conductor channels on the surface having a relatively higher energy surface property for guiding a flow of fluid towards a desired direction.

22. The method of claim 21, wherein the relatively lower energy surface property limits an adhesion of a target analyte on the surface and makes the surface relatively hydrophobic, and the higher energy surface property of the textured structure makes the channels relatively hydrophilic.

23. The method of claim 22, where the textured structure is formed by at least one of laser ablation, sandblasting, etching and calendaring.

24. A method for detecting a target analyte, comprising: providing a capture molecule structure having a ligand end and a polarizable end, wherein when the capture molecule structure is disposed in a carrier fluid the capture molecule structure is a free floating element; providing a target analyte as another free floating element in the carrier fluid, where the ligand end of the capture molecule structure binds to the target analyte and forms a free floating polar conjugate having a positive end and a negative end; aligning the polar conjugate in the carrier fluid in an electric field; and measuring an electrical property of the aligned polar conjugate to detect the target analyte.

25. The method of claim 24, wherein the carrier fluid includes at least one of water, a buffer, a surfactant, a lysing material, a preservative and a body fluid, including at least one of saliva, urine, exhaled breath condensate, blood and sweat.

26. The method of claim 24, wherein the step of measuring comprises pulsing the electric field for a duration and taking a measurement of the electrical property within a period of time after the duration, where the period of time is short enough to allow detecting the target analyte.

27. The method of claim 24, wherein the carrier fluid is a bio fluid sample; the capture molecule structure is provided as a dry powder prior a step of mixing the capture molecule structure with the carrier fluid; and the target analyte is a constituent of the bio fluid sample.

28. The method of claim 24, wherein the carrier fluid is an environmental fluid sample, and the target analyte is a constituent of the environmental fluid sample.

29. A capture molecule conjugate for detecting a target analyte, comprising: an applied-field-reactive capture molecule conjugate having at least one applied-field-responsive end and at least one capture molecule end, wherein each said capture molecule end binds to and captures the target analyte and wherein the binding of each said capture molecule end to the target analyte increases an electrical charge difference between the at least one applied-field-responsive end and the at least one capture molecule end.

30. The capture molecule conjugate of claim 29, A capture molecule conjugate according to claim 27, comprises a linker molecule disposed between the at least one applied-field-responsive end and the at least one capture molecule end, the linker molecule providing the applied-field-reactive capture molecule conjugate with electro-chemical properties wherein when the at least one capture molecule end binds with the target analyte at least one of a polarity and a conductivity of the applied-field-reactive capture molecule conjugate changes.

31. The capture molecule conjugate of claim 29, wherein the at least one applied-field-responsive end comprises at least one of a nanoparticle, a carbon nanotubes, and graphene.

32. The capture molecule conjugate of claim 29, where the at least one capture molecule end comprises at least one of an engineered antibody, an antibody, an aptamer, a nanobody and a nanoCLAMP.

33. The capture molecule conjugate of claim 29, wherein the applied-field-responsive end comprises the linker molecule comprises PEG, and the capture molecule comprises a nanoCLAMP.

34. A method for concentrating a target analyte in an exhaled breath condensate (EBC) sample, comprising the steps of: collecting the EBC sample from the lungs and airways of a test subject, the EBC containing the target analyte; providing a super absorbent polymer in a flow path of the EBC where during the collection, the EBC sample is contacted with the super absorbent polymer, where the super absorbent polymer absorbs a portion of water from the EBC sample and does not absorb the target analyte resulting in a concentration of the target analyte in remaining water in the EBC sample.

35. The method of claim 34, where the super absorbent polymer is provided as a fiber.

36. The method of claim 34, where a selectively permeable membrane is provided downstream in the flow path of the EBC sample from the super absorbent polymer having a pore size configured and dimensioned to allow a portion of water in the EBC sample not absorbed in the super absorbent polymer blend and the target analyte to flow through the selectively permeable membrane and preventing the super absorbent polymer from flowing through the selectively permeable membrane resulting in a concentration of the target analyte in remaining water in the EBC sample.

37. The method of claim 34, where a selectively permeable membrane is provided upstream in the flow path of the EBC sample from the super absorbent polymer having a pore size configured and dimensioned to allow a portion of water in the EBC sample to flow through the selectively permeable membrane to the super absorbent polymer and preventing the target analyte to flow through the selectively permeable membrane resulting in a concentration of the target analyte in remaining water in the EBC sample.

38. An apparatus for testing exhaled breath condensate (EBC) for a target biomarker, comprising:
an EBC collector for converting breath vapor received from the lungs and airways of a test subject into an EBC biosample, the EBC biosample containing the target analyte;
a biomarker concentrator comprising a super absorbent polymer layer in a flow path of the EBC biosample where during the collection, the EBC biosample sample is contacted with the super absorbent polymer layer, where the super absorbent polymer absorbs a portion of water from the EBC biosample sample and does not absorb the target analyte resulting in a concentration of the target analyte in remaining water in the EBC biosample sample; and
a biomarker testing unit for receiving the concentrated EBC biosample and testing the concentrated EBC biosample for a target biomarker.

39. The apparatus of claim 38, wherein the biomarker concentrator further comprises a lysing material incorporated with the super absorbent polymer for releasing the target biomarker from a biological element comprising at least one of a virus, a cell, and a bacteria.

40. The apparatus of claim 38, wherein the biomarker concentrator further comprises a selectively permeable membrane having pores configured and dimensioned to allow the flow of the target biomarker through the selectively permeable membrane towards a testing unit.

41. An electronic biosensor, comprising a substrate having a water absorbing property provided by at least one of a selectively permeable membrane, a super absorbent polymer, a microfluidic material, and a wick; at least two electrodes formed on a top surface of the substrate defining a gap there between; a functionalized detector provided in the gap and comprising an electron transport material and a capture molecule, wherein a target molecule captured by the capture molecule causes a change in at least one of a polarity and conductivity of the electron transport material.

42. The electronic biosensor of claim 41, where the target molecule is an element of a capture molecule conjugate comprising at least a linker and the capture molecule, where the linker bonds the capture molecule to the electron transport material.

43. The electronic biosensor of claim 41, where the electron transport material comprises carboxylated carbon nanotubes incorporated in a layer of super-absorbing polymer, and where the electron transport layer is cast over a porous hydrophilized polypropylene support.

44. The electronic biosensor of claim 43, where the functionalized detector is patterned on the hydrophilized polypropylene support and the electrodes are formed on a top surface of the hydrophilized polypropylene support.

45. A sensor for detecting target molecules in a fluid sample, comprising:
a detection area for receiving the fluid sample comprising the target molecules and having a detection interface functionalized with capture molecules;
a top driving electrode and a bottom driving electrode defining a gap there between; and
a fluid conductor disposed in the gap for conducting the fluid sample through the gap, wherein an electric potential applied to the top and the bottom driving electrode drives the target molecules towards the capture molecules to concentrate the target molecules in a portion of the fluid sample received at the detection interface.

46. The sensor of claim 45, wherein the top and the bottom driving electrodes are disposed upstream of the fluid sample flow from the detection area, wherein the fluid conductor is provided upstream and downstream from the detection area to flow the fluid sample with the target molecules over the detection area, and further comprising a wick for removing excess water from the fluid sample upstream from the detection area.

47. The sensor of claim 45, wherein the top and the bottom driving electrodes are disposed in the detection area; and further comprising a wick for absorbing excess fluid from the sample, wherein the fluid conductor conducts a portion of the fluid sample containing relatively less target molecules through the gap to the wick and another portion of the fluid sample containing relatively more target molecules towards the capture molecules.

48. The sensor of claim 47, wherein the wick includes super absorbent polymer beads having a shell surface, and the electric potential applies a shell charge potential to the shell, where the shell charge potential is an opposite charge as a target molecule charge potential and drives the target molecules away from the super absorbent polymer beads while allowing a portion of the fluid sample to enter through the shell and be absorbed by the beads.

49. The sensor of claim 45, where at least one of the top and the bottom driving electrode comprises a grid electrode having spaces between conductive elements to allow the fluid sample to flow.

50. The sensor of claim 45, wherein the fluid conductor comprises at least one of a capillary channel, a microfluidic material, a super absorbent polymer, and a fluid conductive fibrous sheet.

51. The sensor of claim 45, wherein the sensor comprises a field-effect transistor having a gate disposed in electrical communication with the detection interface functionalized with the capture molecules, and a source and drain on either side of the gate, and wherein at least a portion of at least one of the top and the bottom driving electrode is disposed in the detection area, and where a gate electrode of the sensor comprises at least one of the top and the bottom driving electrode.

52. A method for detecting a target molecule from a fluid sample, comprising the steps of:
receiving the fluid sample comprising the target molecule from a microfluidic channel;
transferring the fluid sample from the microfluidic channel to a detection interface of a sensor, the sensor comprising a detection area for receiving the fluid sample and having the detection interface functionalized with capture molecules, a top driving electrode and a bottom driving electrode defining a gap there between, and a fluid conductor disposed in the gap for conducting the fluid sample through the gap, wherein an electric potential applied to the top and the bottom driving electrode drives the target molecules towards the capture molecules to concentrate the target molecules in a portion of the fluid sample received at the detection interface, the sensor comprising a field-effect transistor having a gate disposed in electrical communication with the detection interface functionalized with the capture molecules, and a source and drain on either side of the gate, and wherein at least a portion of at least one of the top and the bottom driving electrode is disposed in the detection area, and where a gate electrode of the sensor comprises at least one of the top and the bottom driving electrode; and
intermittently applying the electric potential for driving the target molecules and for taking a test reading of a change in an electrical characteristic at the source, drain and gate.

53. A sensor, comprising: a superstrate; at least a first and second electrode formed on the superstrate and defining a gap there between, a detection interface comprising capture molecules provided in the gap; a drainage hole formed in the superstrate near the gap; a selectively permeable membrane provided at the drainage hole for allowing a portion of a fluid sample to pass and for blocking at least some target molecules; and a fluid absorber provided in fluid communication with the selectively permeable membrane to absorb the portion of the fluid sample.

54. A lateral flow assay, comprising: a sample pad provided at a first stage of the lateral flow assay; a selectively permeable membrane provided adjacent to the sample pad for allowing a portion of a fluid sample to pass through the sample pad and for blocking at least some target molecules to accumulate target molecules in another portion of the fluid sample that does not pass through the selectively permeable membrane; a fluid absorber provided in fluid communication with the selectively permeable membrane to absorb the portion of the fluid sample that passes through the selectively permeable membrane; and a dissolvable fluid dam for holding said another portion of the fluid sample having accumulated target molecules from flowing to a next stage of the lateral flow assay.

55. A room scale biosensor, comprising:
an intake for taking in ambient air;
a condenser for cooling the ambient air to condense moisture in the ambient air to a condensate containing water and at least one target molecule;
a condensate testing system for testing the condensate for the at least one target molecule;
wherein the condensate testing system includes a biosensor comprising at least one g-FET biosensor having a detection interface comprising a graphene layer functionalized with capture molecules, wherein the capture molecules are smaller than the Debye screening length.

56. The room scale biosensor of claim 55, further comprising a target molecule concentrator for concentrating the at least one target molecule in the fluid biosample to form a concentrated fluid biosample.

57. The room scale biosensor of claim 56, wherein the target molecule concentrator comprises a selectively permeable barrier for allowing excess water in the fluid biosample to pass through the selectively permeable barrier and block the target molecule in the fluid biosample from passing through the selectively permeable barrier and an excess water absorbing wick for absorbing the excess water passing through the selectively permeable material.

58. The room scale biosensor of claim 56, wherein the target molecule concentrator comprises a super absorbent polymer (SAP) for preferentially absorbing water from the EBC into polymer chains of the super absorbent polymer, wherein the target molecule is not absorbed by the polymer chains and flows along with the EBC through the SAP, wherein as the EBC flows along through the SAP the water content in the EBC is removed to increase the tested sample concentration of the target molecules.

59. The room scale biosensor of claim 58, wherein the biosensor includes the capture molecules designed to bind to at least one of FluA, FluB, SARS N-protein and SARS S-protein.

60. The room scale biosensor of claim 58, further comprising a fluid transfer system for transferring the condensate from the condenser to the condensate testing system, the fluid transfer system comprising at least one of a fluid conductor, a pooling area, and a microfluidics transfer path for controlling a flow of the fluid biosample received from the EBC collector; and further comprising a target molecule releasing material disposed in said at least one of the fluid conductor, pooling area and microfluidics transfer path, wherein the target molecule releasing material includes at least one of a surfactant and a chemical lysing agent.

61. The room scale biosensor of claim 55, wherein the condenser includes a condensate-forming surface comprising a relatively low energy surface property for limiting an adhesion of target molecule to the condensate-forming surface, and further comprising a fluid conductor disposed on the condensate-forming surface, wherein the fluid conductor comprises a textured structure formed on the condensate-forming surface, the textured structure having a relatively higher energy surface property for guiding a flow of the EBC towards a desired direction.

62. The room scale biosensor of claim 55, further comprising a target molecule releasing structure for mechanically lysing at least one of a cell wall, encapsulating structure and viral envelope containing the target molecule, wherein the target molecule releasing structure comprises lysing structures protruding from at least one of the condensate-forming surface and the surface of a flow path of the EBC, where the lysing structures mechanically disrupt at least one of the cell wall, encapsulating structure and vial envelope containing the target molecule.

63. A field-effect transistor sensor circuit for detecting target molecules in a fluid sample, comprising, a semiconductor substrate of one conductivity type and having a source region and a drain region defining there between a channel region of the one conductivity type, the source region and the drain region of an opposite conductivity type; an insulator formed over the channel region, wherein the channel region forms a back gate of the field-effect transistor; a detection area formed over the insulator for receiving the fluid sample and having a detection interface functionalized with capture molecules; a top electrode defining a gap with the detection area and forming a liquid gate electrode of the transistor; a fluid conductor disposed in the gap for conducting the fluid sample through the gap; and a driving circuit for applying an electric potential of one polarity to the top electrode and of the opposite polarity to the back gate, wherein the electric potential drives the target molecules towards the capture molecules to concentrate the target molecules in a portion of the fluid sample received at the detection interface.

64. The field-effect transistor sensor circuit of claim 63, further comprising a detection circuit for applying a voltage to the source region and at least one of the back gate and the top electrode, and detecting a change in current through the drain region dependent on a binding of the target molecules with the capture molecules.

65. The field-effect transistor sensor circuit of claim 63, wherein the driving circuit applies the electric potential cyclically with the detection circuit detecting the change in the current.

66. The field-effect transistor sensor circuit of claim 63, wherein a portion of the capture molecules are immobilized on the detection interface at a greater distance from a top surface of the detection interface than another portion of the capture molecules immobilized on the detection interface.

67. The field-effect transistor sensor circuit of claim 63, wherein the driving circuit reverses polarity of the applied electric potential to drive non-target molecules from the detection area while target molecule captured by capture molecules immobilized on the detection interface at a greater distance are retained in the detection area.

68. The field-effect transistor sensor circuit of claim 63, wherein the detection interface comprises at least one of graphene, nanoparticles, aligned carbon nanotubes, vacuum deposited conductive material, transferred conductive material, a semiconductor, a metal, and a screen-printed conductive ink.

69. The field-effect transistor sensor circuit of claim 63, where the capture molecules include at least one of antibodies, engineered antibodies, aptamers, nanoCLAMPS, and CRISPR Cas protein conjugates.

70. The field-effect transistor sensor circuit of claim 63, wherein the capture molecules includes a catalytically inactive CRISPR Cas protein protein conjugated with a guide RNA and immobilized on the graphene detection interface.

71. A packaged biosensor semiconductor device, comprising a semiconductor die including at least a source region and a drain region defining there between a channel region; an insulator formed over the channel region, wherein the channel region forms a back gate of the field-effect transistor; a detection area disposed over the insulator for receiving the fluid sample and having a detection interface functionalized with capture molecules; a lead frame including a die pad for receiving the semiconductor die with leads for conducting electrical signals from the source, channel and drain regions to an external electronic circuit; a base barrier section encapsulating the semiconductor substrate leaving the detection interface exposed; and a top barrier section having a detection well forming an opening disposed over the exposed detection interface for receiving a fluid sample.

72. The packaged biosensor semiconductor device of claim 71, A packaged biosensor semiconductor device according to claim 68; further comprising a top gate lead having an end provided at a top surface of the top barrier section for connecting to a liquid gate electrode and another end provided for connecting the liquid gate electrode to the external electronic circuit, wherein the liquid gate electrode defines a gap with the detection region and for being in electrical communication with a fluid sample when the fluid sample is disposed in the detection well.

73. The packaged biosensor semiconductor device of claim 71, wherein target molecules in the fluid sample are detected by a change in electrical characteristics occurring at one or more of the source, drain, back gate and liquid gate electrode.

74. The packaged biosensor semiconductor device of claim 71, where the capture molecules include at least one of antibodies, engineered antibodies, aptamers, nanoCLAMPS, and CRISPR Cas protein conjugates.

75. The packaged biosensor semiconductor device of claim 71, wherein the capture molecules includes a catalytically inactive CRISPR Cas protein protein conjugated with a guide RNA and immobilized on the graphene detection interface.

76. The packaged biosensor semiconductor device of claim 71, further comprising a z-axis conductive adhesive for making electrical and mechanical connections between between the leads of the packaged IC semiconductor device, the corresponding lead lines and connection pads of an external electrical circuits, where the z-axis conductive adhesive includes a manually removable protective film for protecting the z-axis conductive adhesive until placement of the packaged field-effect transistor sensor in electrical and mechanical connection with the external electrical circuit.

77. The packaged biosensor semiconductor device of claim 71, further comprising additional electronic semiconductor circuit elements encapsulated along with the field-effect transistor sensor forming a packaged integrated circuit semiconductor device.
